(19) United States
(12) Patent Application Publication
(10) Pub. No.: US 2016/0237160 A1
(43) Pub. Date: Aug. 18, 2016

(54) ANTI-TWEAKR ANTIBODIES AND USES THEREOF

(71) Applicant: BAYER PHARMA AKTIENGESELLSCHAFT, Berlin (DE)

(72) Inventors: Christian VOTSMEIER, Köln (DE); Stefanie HAMMER, Berlin (DE); Uwe GRITZAN, Köln (DE); Sandra BERNDT, Hohen Neuendorf (DE); Dmitry ZUBOV, Remscheid (DE); Lars LINDEN, Gevelsberg (DE); Sven CHRISTIAN, Berlin (DE); Axel HARRENGA, Wuppertal (DE); Jörg BIRKENFELD, Frankfurt am Main (DE); Christoph FREIBERG, Wuppertal (DE); Sven GOLFIER, Berlin (DE); Andrea EICKER, Mönchengladbach (DE); Simone GREVEN, Dormagen (DE); Beatrix STELTE-LUDWIG, Wülfrath (DE); Marian RASCHKE, Berlin (DE)

(73) Assignee: BAYER PHARMA AKTIENGESELLSCHAFT, Berlin (DE)

(21) Appl. No.: 14/898,455

(22) PCT Filed: Jun. 12, 2014
(86) PCT No.: PCT/EP2014/062207

§ 371 Date: Dec. 14, 2015

(30) Foreign Application Priority Data
Jun. 14, 2013 (EP) 13172111.0

(51) Int. Cl. C07K 16/28; A61K 49/00; A61K 31/44; A61K 31/4745; A61K 31/282; A61K 31/513; A61K 47/48; A61K 39/395

(52) U.S. Cl. CPC C07K 16/2878; A61K 47/48569; A61K 49/0058; A61K 39/39558; A61K 31/4745; A61K 31/282; A61K 31/513; A61K 31/44; C07K 16/2863; C07K 2317/75; C07K 2317/565; C07K 2317/622; C07K 2317/55; C07K 2317/54; C07K 2317/21; C07K 2317/24; C07K 2317/73; C07K 2317/34; A61K 2039/505

(57) ABSTRACT

The present invention provides recombinant antigen-binding regions and antibodies and functional fragments containing such antigen-binding regions that are specific for the TWEAKR (TNFRSF12A, FN14). The antibodies, accordingly, can be used to treat tumors and other disorders and conditions associated with expression of the TWEAKR. The invention also provides nucleic acid sequences encoding the foregoing antibodies, vectors containing the same, pharmaceutical compositions and kits with instructions for us.

The present invention provides recombinant antigen-binding regions and antibodies and functional fragments containing such antigen-binding regions that are specific for the TWEAKR (TNFRSF12A, FN14).

The antibodies, accordingly, can be used to treat tumors and other disorders and conditions associated with expression of the TWEAKR. The invention also provides nucleic acid sequences encoding the foregoing antibodies, vectors containing the same, pharmaceutical compositions and kits with instructions for use.

### BACKGROUND OF THE INVENTION

Antibody-based therapy is proving very effective in the treatment of various cancers, including solid tumors. For example, HERCEPTIN® has been used successfully to treat breast cancer and RITUXAN® is effective in B-cell related cancer types. Central to the development of a novel successful antibody-based therapy is the isolation of antibodies against cell-surface proteins found to be preferentially expressed on tumor cells that are able to functionally modify the activity of the corresponding receptor.

Tumor necrosis factor (TNF) like weak inducer of apoptosis (TWEAK) and the TWEAK receptor (TWEAKR, alias TNFRSF12A, FN14, CD266; Swiss Prot Acc. Q9NP84, NP_057723) are a TNF superfamily ligand-receptor pair involved in inflammation, proliferation, invasion, migration, differentiation, apoptosis and angiogenesis (Winkles J A, Nat Rev Drug Discov. 2008 May; 7(5):411-25; Michaelson J S and Burkly L C, Results Probl Cell Differ. 2009; 49:145-60). TWEAK binds to TWEAKR with an affinity of 0.8-2.4 nM and is the only member of the TNF family that binds this receptor (Wiley S R et al., Immunity. 2001 Nov.; 15(5):837-46). The TWEAKR is expressed at relatively low levels in normal tissues, but is markedly increased locally in injured tissues, where it has a role in tissue remodeling (Winkles J A, Nat Rev Drug Discov. 2008 May; 7(5):411-25; Zhou et al., Mol Cancer Ther. 2011 Jul.; 10(7):1276-88; Burkly L C et al., Immunol Rev. 2011 November; 244(1):99-114). TWEAKR signaling is involved in processes as wound healing, chronic autoimmune disease and acute ischemic stroke (Burkly L C et al., Immunol Rev. 2011 November; 244(1):99-114). In addition, the TWEAKR is highly expressed in various solid tumor types as for example pancreatic cancer, non-small-cell-lung-cancer (NSCLC), colorectal cancer (CRC), breast cancer, renal cancer, head and neck cancer, esophageal cancer, bladder cancer, hepatocellular carcinoma, ovarian cancer, melanoma as well as liver and bone metastasis (Culp P et al., Clin Cancer Res. 2010 Jan. 15; 16(2):497-508; Zhou H et al., J Invest Dermatol. 2013 April; 133(4):1052-62). Association of increased TWEAKR expression and higher tumor grade and/or poor prognosis has been described in brain (Tran N L et al., Cancer Res. 2006 Oct. 1; 66(19):9535-42), breast (Willis A L et al., Mol Cancer Res. 2008 May; 6(5):725-34; Wang J et al., Histol Histopathol. 2013 Jan. 9 [Epub ahead of print]), esophageal (Watts G S et al., Int J Cancer. 2007 Nov. 15; 121(10):2132-9 2007), prostate (Huang M et al., Carcinogenesis. 2011 November; 32(11):1589-96), gastric (Kwon O H et al., Cancer Lett. 2012 Jan. 1; 314(1):73-81), neuroblastoma (Pettersen I et al., Int J Oncol. 2013 April; 42(4):1239-48) and bladder cancer (Shimada K et al., Clin Cancer Res. 2012 Oct. 1; 18(19):5247-55).

Expression of TWEAKR is induced by growth factors as FGF, PDGF and VEGF (Winkles J A, Nat Rev Drug Discov. 2008 May; 7(5):411-25). In line with this observation, it has been shown that TWEAKR expression correlates with EGFR overexpression or activation in NSCLC (Whitsett T G et al., Am J Pathol. 2012 July; 181(1):111-20) and HER2 expression in breast cancer (Wang J et al., Histol Histopathol. 2013 Jan. 9 [Epub ahead of print]; Chao D T et al., J Cancer Res Clin Oncol. 2013 February; 139(2):315-25).

Activation of the TWEAKR by TWEAK leads to recruitment of TNF-receptor associated factors (TRAF) to the intracellular binding domain resulting in prolonged NF-κB activation via the canonical and non-canonical NF-κB pathway and induction of cytokine secretion as IL-8 and MCP-1 (reviewed in Michaelson J S and Burkly L C, Results Probl Cell Differ. 2009; 49:145-60). This is well in accordance with the described pro-inflammatory role of the TWEAK/TWEAKR pathway. However, the signaling pathways responsible for cell killing via TWEAKR are less clear, as the TWEAKR lacks a characteristic “death domain”. In some tumor cell lines (Kym-1, SKOV-3, OVCAR) it induces apoptosis through TNF and the recruitment of TRAF2, followed by lysosomal degradation of the resulting TRAF2-cIAP complex (Nakayama M. et al, J Immunol. 2002 Jan. 15; 168(2):734-43; Schneider P et al, Eur J Immunol. 1999 Jun.; 29(6):1785-92; Vince J E et al, J Cell Biol. 2008 Jul. 14; 182(1):171-84). In other cell lines (HSC3, HT-29, KATO-III) TWEAK induced apoptosis is reported to be TNF independent (Nakayama M et al, J Immunol. 2003 Jan. 1; 170(1):341-8; Wilson C A et al, Cell Death Differ. 2002 Dec.; 9(12):1321-33). In a recent report induction of apoptosis by TWEAK was shown to be dependent on the stimulation of Stat-1 phosphorylation as treatment with a JAK-inhibitor abolished the ability of TWEAK to increase caspase3/7 activation in WiDr cells (Chapman M S et al, Cytokine. 2013 January; 61(1):210-7).

Several studies validated TWEAKR as an oncologic target. Michaelson et al have shown that the administration of TWEAK reduces tumor growth in murine xenograft models (Michaelson J S et al, MAbs. 2011 Jul.-Aug.; 3(4):362-75). This anti-tumor effect has been imitated by several groups with agonistic anti-TWEAKR antibodies. Potential drug candidates, namely BIIB0036/P4A8 (Michaelson J S et al, MAbs. 2011 Jul.-Aug.; 3(4):362-75) and PDL-192, (Culp P A et al, Clin Cancer Res. 2010 Jan. 15; 16(2):497-508) have been generated by immunization of mice and subsequent clonal selection and humanization.

PDL-192 binds to the TWEAKR with a binding affinity of 5.5 nM (Culp P A et al, Clin Cancer Res. 2010 Jan. 15; 16(2):497-508) and inhibits the growth of several TWEAKR expressing cancer cell lines. Yet, in comparison to TWEAK ligand PDL-192 was shown to be less potent in proliferation and apoptosis assays with respect to EC/IC50 and only reached reduced efficacy (Vₘₐₓ) of caspase 3/7 activation (Culp P A et al, Clin Cancer Res. 2010 Jan. 15; 16(2):497-508). Profiling in a larger panel of breast cancer cell lines confirmed the only modest anti-proliferative activity of monomeric PDL-192 (Culp P A et al, Clin Cancer Res. 2010 Jan. 15; 16(2):497-508; Chao D T et al, J Cancer Res Clin Oncol. 2013 February; 139(2):315-25) with only 5 of 27 cell lines responding with >20% of proliferation inhibition. Anti-proliferative activity of the antibody is slightly enhanced by cross-linking or immobilization of the antibody. In addition, PDL-192 exhibits ADCC and the anti-tumor activity described in xenograft models is thought to be a mixture of ADCC and tumor cell growth inhibition effects (Culp P A et al, Clin Cancer Res. 2010 Jan. 15; 16(2):497-508). A further limitation of PDL-192 is the lack of species cross-reactivity, especially mouse and rat, not allowing e.g. assessment of common pre-clinical studies as toxicological studies.

The second agonistic anti-TWEAKR antibody described as drug candidate, BIIB036/P4A8 binds to TWEAKR with an affinity of 1.7 nM which is in a similar range as the endogenous ligand TWEAK (Michaelson J S et al, MAbs. 2011 Jul.-Aug.; 3(4):362-75). This antibody is shown to induce activation of NF-κB and cytokine release in cancer cells, albeit significantly less efficacious compared to Fc-TWEAK, a hIgG1 Fc-fusion of soluble TWEAK (aa 106-249) with similar activity as recombinant soluble TWEAK (Michaelson J S et al., Oncogene. 2005 Apr. 14; 24(16):2613-24). The same holds true in cell proliferation assays as well as for induction of apoptosis as shown in a TUNEL staining after treatment of cells with antibodies, where potency of BIIB036/P4A8 is also significantly decreased compared to Fc-TWEAK. Anti-proliferative activity increases after multimerization of the antibody, but also the multimerized form is still less efficacious as compared to recombinant Fc-TWEAK. In contrast, BIIB036/P4A8 is a potent inducer of ADCC and anti-tumor activity in xenograft models was shown to be largely dependent on Fc effector function.

Besides both drug candidates several murine antibodies have been described that would need antibody engineering for humanization to be useful for a human therapy. The first anti-TWEAKR antibodies with anti-proliferative activity on cancer cells were antibodies Item 1-4 described by Nakayama et al. (Nakayama M et al, Biochem Biophys Res Commun. 2003 Jul. 11; 306(4):819-25). These antibodies, however, harbor only relatively weak agonistic activity and were shown to act as partial agonists/antagonists with regard to TWEAK mediated TWEAKR activation. Antibodies 136.1 and 18.3.3 (WO2009/020933) show higher affinity binding compared to TWEAK ligand, which does not translate in more efficacious caspase activation. Antibodies P3G5 and P2D3 (WO2009/140177) induce cytokine release in cancer cells significantly less efficacious compared to Fc-TWEAK. To summarize, TWEAKR agonistic activity with regard to induction of apoptosis and inhibition of proliferation of the anti-TWEAKR antibodies described in the art is limited and does not reach or exceed the efficacy of the endogenous ligand TWEAK. This lack of agonistic activity is not due to a decreased affinity as these antibodies bind to the TWEAKR with affinities in a similar range as compared to the endogenous ligand TWEAK (Michaelson J S et al, MAbs. 2011 Jul.-Aug.; 3(4):362-75; Culp P A et al, Clin Cancer Res. 2010 Jan. 15; 16(2):497-508) and also antibodies with higher binding affinity do not necessarily exhibit more potent signaling activity (Culp P A et al, Clin Cancer Res. 2010 Jan. 15; 16(2):497-508). Anti-tumor activity of the antibodies described previously is shown to be dependent on Fc effector function and ADCC is shown to play a significant role for the in vivo efficacy in mouse models. The contribution of ADCC and in vivo cross linking via Fc-Fc receptor (FcR) interactions to anti-tumor activity in solid tumors in the clinic, however, is still not clear, given the challenge of antibody and immune effector cell penetration into solid tumors (Culp P A et al, Clin Cancer Res. 2010 Jan. 15; 16(2):497-508). Additionally, patients carrying low-affinity alleles of FcγRIIIA would exhibit a reduced benefit from the treatment due to lower Fc-FcR interaction capacity (Varchetta S et al, Cancer Res. 2007 Dec. 15; 67(24): 11991-9).

Thus, developable human antibodies with strong intrinsic capacity to induce cancer cell apoptosis and growth inhibition by hyper-activation of the TWEAKR to the same or even higher extend as compared to the endogenous ligand TWEAK are highly demanded. As induction of apoptosis and inhibition of proliferation is since many years a valid concept in inducing anti-tumor response in patients (Hanahan D and Weinberg R A, Cell. 2000 Jan. 7; 100(1):57-70; Kim R et al, Cancer Chemother Pharmacol. 2002 November; 50(5):343-52; Fesik S W, Nat Rev Cancer. 2005 Nov.; 5(11):876-855) these antibodies are expected to show increased anti-tumor activity in solid tumors in human and are therefore promising drug candidates for the treatment of cancer.

### SUMMARY OF THE INVENTION

This invention is related to antibodies, or antigen-binding antibody fragments thereof, or variants thereof which lead to strong activation of the TWEAKR, thus leading to a strong induction of apoptosis in various cancer cells showing overexpression of the TWEAKR. Induction of cancer cell apoptosis by the antibodies described herein is more efficacious compared to all antibodies described in the art (e.g. PDL-192 or BIIB0036/P4A8; e.g. require the addition of a cross-linking agent). The unique property of the antibodies of this invention is based on a novel binding epitope characterized by selective binding of the antibodies to amino acid at position 47 (D47) of TWEAKR (SEQ ID NO:169; and see FIG. 1).

The antibodies of the invention are thus suitable for the treatment of cancer as well as metastases thereof, in particular TWEAKR expressing tumors, such as colorectal cancer, non-small-cell lung cancer (NSCLC), head and neck cancer, esophageal cancer, melanoma, hepatocellular carcinoma, bladder cancer, gastric cancer, breast cancer, pancreatic cancer, renal cell carcinoma, prostate cancer, ovarian cancer and cervical cancer.

The invention describes antibodies that are distinguished from existing anti-TWEAKR antibodies in that they induce strong activation of cancer cell apoptosis, at superior levels as compared to the endogenous ligand TWEAK in most cell lines. The antibodies of the invention or antigen-binding fragments thereof a) strongly activate the TWEAKR, b) induce apoptosis in cancer cells, c) induce cytokine secretion from cancer cells, d) all together resulting in anti-tumor activity of the antibodies in in vivo tumor experiments, e) additionally the antibodies lead to internalization of the TWEAKR and inhibition of cancer cell proliferation when incubated with saporine-conjugated secondary antibodies in experimental conditions where the antibody alone has no effect, f) are crossreactive to several species. These and other objects of the invention are more fully described herein.

An antibody of the invention might be co-administered with known medicaments, and in some instances the antibody might itself be modified. For example, an antibody could be conjugated to a cytotoxic agent, immunotoxin, toxophore or radioisotope to potentially further increase efficacy.

The invention further provides antibodies which constitute a tool for diagnosis of malignant or dysplastic conditions in which TWEAKR expression is elevated compared to normal tissue. Provided are anti-TWEAKR antibodies conjugated to a detectable marker. Preferred markers are a radiolabel, an enzyme, a chromophore or a fluorescer.

The invention is also related to polynucleotides encoding the antibodies of the invention or antigen-binding fragments thereof, cells expressing the antibodies of the invention or antigen-binding fragments thereof, methods for producing the antibodies of the invention or antigen-binding fragments thereof, methods for inhibiting the growth of dysplastic cells using the antibodies of the invention or antigen-binding fragments thereof, and methods for treating and detecting cancer using the antibodies of the invention or antigen-binding fragments thereof.

The invention is also related to isolated nucleic acid sequences, each of which can encode an aforementioned antibody or antigen-binding fragment thereof that is specific for an epitope of TWEAKR. Nucleic acids of the invention are suitable for recombinant production of antibodies or antigen-binding antibody fragments. Thus, the invention also relates to vectors and host cells containing a nucleic acid sequence of the invention.

Compositions of the invention may be used for therapeutic or prophylactic applications. The invention, therefore, includes a pharmaceutical composition comprising an inventive antibody or antigen-binding fragment thereof and a pharmaceutically acceptable carrier or excipient therefore. In a related aspect, the invention provides a method for treating a disorder or condition associated with the undesired presence of TWEAKR expressing cells. In a preferred embodiment the aforementioned disorder is cancer. Such method contains the steps of administering to a subject in need thereof an effective amount of the pharmaceutical composition that contains an inventive antibody as described or contemplated herein.

The invention also provides instructions for using an antibody library to isolate one or more members of such library that binds specifically to TWEAKR.

### DESCRIPTION OF THE FIGURES

FIG. 1: Alignment of TWEAKR cysteine rich domain (aa 34-68) of different species. (Numbers indicate amino acid position in full length construct inclusive signal sequence; SEQ ID NO: 169)

FIG. 2: A—Schematic diagram of the structure of TWEAKR (SEQ ID NO:169). The diagram shows the extracellular domain (aa 28-80) (SEQ ID NO:168) including the cysteine rich domain (36-67), the transmembrane domain—TM (81-101), and the intracellular domain (102-129). TPP-2202—the full ectodomain (28-80) fused to the Fc domain of hIgG1. TPP-2203—Extracellular domain with N- and C-terminal truncation (34-68) fused to the Fc domain of hIgG1. Disulfide bridges Cys36-Cys49, Cys52-Cys67 and Cys55-Cys64 are indicated by black bars. N-terminally, TPP-2203 contains two amino acids and C-terminally, one amino acid more compared to the pure cysteine rich domain to ensure proper folding. TPP-1984—Extracellular domain with C-terminal truncation (28-68) fused to HIS6 tag. All three constructs show comparable binding to the antibodies of the invention and PDL-192(TPP-1104). P4A8(TPP-1324) does only bind to the full extracellular domain (TPP-2202).

B—Amino acid sequence of extracellular domain: aa46 has been published to be essential for TWEAK ligand binding, aa47 has been characterized to be essential for binding of the antibodies of this invention.

FIG. 3: Interaction of TWEAKR ectodomain with antibodies of the invention and reference antibodies. Shown is the result of an ELISA with TWEAKR-Fc fusion protein (TPP-2202) coating (1 μg/ml) and 0.08 μg/ml (open bars) and 0.3 μg/ml (filled bars) of biotinylated IgG as soluble binding partner. Detection was done with Streptavidin-HRP and Amplex-Red substrate. Y is “ELISA signal intensity [Rfu]”; X are “antibody constructs tested”: a is “TPP-2090”; b is “TPP-2084”; c is “PDL-192(TPP-1104)”; d is “P4A8(TPP-1324)”; e is “P3G5(TPP-2195)”; f is “136.1(TPP-2194)”; h is “ITEM1”; i is “ITEM4”; j is a murine isotype control; k is a human isotype control. All tested antibodies show saturated binding with a concentration of 80 ng/ml.

FIG. 4: Interaction of TWEAKR cysteine rich domain with antibodies of the invention and reference antibodies. Shown is the result of an ELISA with TWEAKR(34-68)-Fc fusion protein (TPP-2203) coating (1 μg/ml) and 0.08 μg/ml (open bars) and 0.3 μg/ml (filled bars) of biotinylated IgG as soluble binding partner. Detection was done with Streptavidin-HRP and Amplex-Red substrate. Y is “ELISA signal intensity [Rfu]”; X are “antibody constructs tested”: a is “TPP-2090”; b is “TPP-2084”; c is “PDL-192(TPP-1104)”; d is “P4A8(TPP-1324)”; e is “P3G5(TPP-2195)”; f is “136.1(TPP-2194)”; h is “ITEM1”; i is “ITEM4”; j is a murine isotype control; k is a human isotype control. The antibodies of the invention bind to the cysteine rich domain.

FIG. 5: Interaction of TWEAKR(28-68) with antibodies of the invention and reference antibodies. Shown is the result of an ELISA with TWEAKR(28-68)-HIS (TPP-1984) coating (1 μg/ml) and 0.08 μg/ml (open bars) and 0.3 μg/ml (filled bars) of biotinylated IgG as soluble binding partner. Detection was done with Streptavidin-HRP and Amplex-Red substrate. Y is“ELISA signal intensity [Rfu]”; X are “antibody constructs tested”: a is “TPP-2090”; b is “TPP-2084”; c is “PDL-192(TPP-1104)”; d is “P4A8(TPP-1324)”; e is “P3G5(TPP-2195)”; f is “136.1(TPP-2194)”; h is “ITEM1”; i is “ITEM4”; j is a murine isotype control; k is a human isotype control. The antibodies of the invention bind to the cysteine rich domain. Antibodies P4A8(TPP-1324), P3G5(TPP-2195), ITEM-1 and ITEM-4 show impaired binding.

FIG. 6: A—Alanine scan of cysteine rich domain. Muteins of TWEAKR(34-68)-Fc were analyzed for PDL-192(TPP-1104) (X) and TPP-2090 (Y) binding. S37A, R38A, S40A, W42A, S43A, D45A, D47A, K48A, D51A, S54A, R56A, R58A, P59A, H60A, S61A, D62A, F63A and L65A muteins were expressed in HEK293 cells (black diamonds). PDL-192(TPP-1104) and TPP-2090 were coated (1 μg/ml) and an eight-fold diluted supernatant of the HEK293 fermentation broth was added for TWEAKR mutein binding. X is “ELISA intensity of PDL-192(TPP-1104) interaction [Rfu]”, Y is “ELISA intensity of TPP-2090 interaction [Rfu]”. TPP-2090 (Y) shows impaired binding for the D47A TWEAKR mutein (closed box) and PDL-192(TPP-1104) (X) shows impaired binding to R56A (dotted box).

B—Y is “% binding normalized by wt binding signal [%]”, 1 is “TPP-2090”; 2 is “PDL-192(TPP-1104)”; 3 is “P4A8(TPP-1324)”. Antibodies were coated (1 μg/ml), TWEAKR variant was added at 250 ng/ml, detection via anti-HIS HRP. TTP-2090 shows less than 5% binding compared to the WT construct.
C—Y is “% binding normalized by wt binding signal [%]”, 1 is “TPP-2090”; 2 is “TPP-2149”, 3 is “TPP-2093”; 4 is “TPP-2148”; 5 is “TPP-2084”; 6 is “TPP-2077”; 7 is “TPP-1538”; 8 is “TPP-883”; 9 is “TPP-1854”; 10 is “TPP-1853”; 11 is “TPP-1857”; 12 is “TPP-1858”; 13 is “PDL-192(TPP-1104)”. Antibodies were coated (1 μg/ml), TWEAKR variant was added 250 ng/ml, detection via anti-HIS HRP. All variants despite PDL-192 show less than 5% binding compared to the WT construct.

FIG. 7: NMR structure of TWEAKR ectodomain as published by Pellegrini et al (FEBS 280:1818-1829). TWEAK binding depends on L46 (Pellegrini et al), TPP-2090 binding on D47 and PDL-192 binding on R56. PDL-192 binds opposite of the TWEAK ligand binding site, TPP-2090 binds directly to the TWEAK ligand site.

FIG. 8: To differentiate binding epitopes of antibodies of the invention and of reference antibodies competition experiments were performed. A lack of a second binding event after injection of the 2nd antibody indicates clear competition within a respective antibody pair. Non competing antibody pairs showed clear binding signal over background after 2nd antibody injection. In addition the investigation of self-competition (1st & 2nd antibody identical) was monitored as an internal system control. (−) no 2^{nd} binding detected; (+) 2^{nd} binding. The antibodies of the invention compete with all tested antibodies.

FIG. 9: To differentiate binding epitopes of antibodies of the invention and of reference antibodies competition experiments were performed. In general all analyzed anti-TWEAKR antibodies could be clustered into three distinct “competition groups”. One group contains exclusively TPP-2084 and TPP-2090, both showing competition to all other tested members. These other members could be split into two separate sets of antibodies, which do not show any competition between each other. Both antibodies of the invention bind to a new and unique epitope.

FIG. 10: Homology tree of all 29 known TNF receptor superfamily members. The closest homologs TNFRSF13C and TNFRSF17 have only about 30% sequence identity.

FIG. 11: Binding ELISA with all 29 TNF receptor superfamily members for selectivity assessment of TPP-2090. Shown is the result of an ELISA: Y is “ELISA signal intensity [Rfu]”; X are “TNF receptor superfamily proteins tested (Fc-fusion proteins)”: 1 is “TWEAKR”; 2 is “TWEAKR”; 3 is “Apo-3”; 4 is “Trail-R1”; 5 is “Trail-R2”; 6 is “CD385”; 7 is “CD95”; 8 is “Rank”; 9 is “TNF-R1”; 10 is “TNF-R2”; 11 is “BAFF-R”; 12 is “DcR3”; 13 is “BCMA”; 14 is “TACI”; 15 is “OX40”; 16 is “CD30”; 17 is “CD27”; 18 is “CD40”; 19 is “Osteoprotegerin”; 20 is “EDAR”; 21 is “GITR”; 22 is “HVEM”; 23 is “NGF R”; 24 is “Trail R3”; 25 is “Lymphotioxin B R”; 26 is “Trail R4”; 27 is “EDA2R”; 28 is “TROY”; 29 is “RELT”; 30 is “4-1BB”. In (1) 300 pM TPP-2090 were employed, in (2) 75 nM. TPP-2090 binds at a very low concentration of 300 pM (1) and at a high concentration of 75 nM (2) in saturation to TWEAKR. For binding analysis to all other TNF receptor superfamily members (3-30) 75 nM TPP-2090 were used. TPP-2090 binds selectively to TWEAKR.

FIG. 12: FACS analysis for binding of anti-TWEAKR antibodies to HT-29 cells. Y is “background corrected Geo-Mean of FACS signal [au]”. Shown is the fluorescence after FACS analysis of HT-29 cells incubated with the antibodies as indicated at 10 μg/ml subtracted by the Geo-Mean of fluorescence of HT-29 cells incubated with the secondary antibody alone. Antibodies of the invention (TPP-1538, TPP-2084, TPP-2090) show lower cellular binding at this concentration as compared to known antibodies [PDL-192(TPP-1104) and P4A8(TPP-1324)].

FIG. 13: Caspase 3/7 activation by anti-TWEAKR antibodies in HT-29 cells. X is “anti-TWEAKR antibodies tested [μg/ml]”; Y is “relative light units [RLU]”. HT-29 cells were incubated with anti-TWEAKR antibodies at different concentrations as indicated (0.03-300 μg/ml) for 24 h in the presence of IFNgamma. Caspase 3/7 activity measured as luminescence by the Caspase 3/7 Glo reagent (Promega) was plotted against the antibody concentrations. Average values of 1-3 representative experiments performed in triplicates are shown including standard deviations. Filled symbols show antibodies of the invention, open symbols known antibodies [PDL-192(TPP-1104); P4A8(TPP-1324), 136.1(TPP-2194)]. The antibodies of the invention (TPP-1538, TPP-1854, TPP-2084, TPP-2090) display a stronger efficacy to induce Caspase 3/7 activation compared to the known antibodies [PDL-192(TPP-1104); P4A8(TPP-1324) and 136.1(TPP-2194)].

FIG. 14: Antiproliferative activity of anti-TWEAKR antibodies in WiDr (A) and 786-O (B) cells. X is “anti-TWEAKR antibodies tested [μg/ml]”; Y is “Cell proliferation related to proliferation of untreated control cells [%]”. Cells were incubated with anti-TWEAKR antibodies at different concentrations as indicated (0.03-300 μg/ml) for 96 h (WiDr cells absence, 786-O cells in the presence of IFN gamma). Average values of a representative experiment performed in triplicates are shown and standard deviations are indicated by error bars. Filled symbols: antibodies of the invention, open symbols known antibodies [PDL-192(TPP-1104) and P4A8(TPP-1324]. The antibodies of the invention (TPP-1538, TPP-1854, TPP-2084, TPP-2090) display a stronger efficacy to inhibit cellular proliferation compared to the known antibodies [PDL-192(TPP-1104) and P4A8(TPP-1324].

FIG. 15: IL-8 secretion induced by anti-TWEAKR antibodies in A375 cells. X is “anti-TWEAKR antibodies tested [μg/ml]”; Y is “IL-8 levels [pg/ml]”. A375 cells were incubated with anti-TWEAKR antibodies at different concentrations as indicated (0.03-300 μg/ml). Levels of IL-8 were determined in the supernatant of the cells after 24 h treatment (and plotted against the used antibody concentrations. Average values of 1-3 representative experiments performed in triplicates are shown including standard deviations. Filled symbols show antibodies of the invention, open symbols known antibodies [PDL-192(TPP-1104); P4A8(TPP-1324), 136.1(TPP-2194)], and treatment with an isotype control antibody is indicated (C). The antibodies of the invention (TPP-1538, TPP-1854, TPP-2084, TPP-2090) display a stronger efficacy to induce IL-8 secretion from A375 cells compared to the known antibodies [PDL-192(TPP-1104), P4A8(TPP-1324), 136.1(TPP-2194)].

FIG. 16: Human IL-8 secretion induced by anti-TWEAKR antibodies in xenografts in mice.

A: WiDr xenograft tumor bearing mice were treated with a single dose of 3 mg/kg TPP-2090 (open symbols) or vehicle (C—filled symbols) and levels of human IL-8 (IL-8 pg/ml) determined at different time points after treatment in the plasma of tumor bearing mice. X is “hours after treatment [h]”; Y is “11-8 level [pg/ml]”. Results from 3 animals per group are indicated, error bars represent standard deviations. Human IL-8 secretion is specifically induced after treatment with TPP-2090 in WiDr tumor bearing mice in a time dependent manner.
B: A375 tumor bearing (filled symbols) or non-tumor bearing (open symbols) mice were treated with a single dose of 10 mg/kg TPP-1538, vehicle or an isotype control antibody. C1 is “vehicle control”; C2 is “isotype control antibody”; Y is “Level of human 11-8 [pg/ml]”. Levels of human IL-8 were determined in the serum of 4 mice per group 7 h after treatment are shown. IL-8 secretion is specifically induced in A375 tumor bearing mice by TPP-1538 but not in equally treated tumor free animals.

FIG. 17: Microscopic evaluation of the time course of specific internalization of TWEAKR upon antibody binding to endogenous TWEAKR expressing cells (InCell Analyzer). Internalization of TPP-1538 and TPP-2090 was investigated on renal cancer cell line 786-O. Granule count/cell after treatment with antibodies of the invention (at 1/μg/ml) or isotype control C—at 5 μg/ml) is plotted for different incubation times as indicated (X is “time [min]”; Y is “granule count/cell [quantity]”). Antibodies of the invention (TPP-1538, TPP-2090) show rapid and specific internalization in TWEAKR expressing cells.

FIG. 18: Inhibition of 786-O cell proliferation by anti-TWEAKR antibodies after incubation with saporine-conjugated secondary antibodies (Hum-Zap Assay). 786-O cells were incubated with TWEAKR or isotype control antibodies in the presence or absence of saporine-conjugated secondary antibodies at 10 nM antibody concentration for 48 h (in the absence of IFN gamma). X is “antibody variant tested”, a is “vehicle control”, b is “isotype control antibody”, c is “TPP-2084”, d is “TPP-2090”; Y is “cell proliferation compared to untreated control cells [%]”. Cell proliferation compared to untreated control cells was plotted for 786-O cells treated with different antibodies in the presence (open bars) or absence (filled bars) of saporine-conjugated secondary antibodies. Results from one representative experiment in triplicates are shown and standard deviations indicated by error bars. At the experimental conditions used only antibodies of the invention (TPP-2084, TPP-2090) in the presence of saporine-coupled secondary antibodies inhibit proliferation of 786-O cells almost completely. Thus, the anti-proliferative effect observed from the anti-TWEAKR antibodies in the presence of saporine-conjugated secondary antibodies is a result of specific internalization of the saporine after binding of the antibody-complexes to TWEAKR expressing cells.

FIG. 19: Efficacy of anti-TWEAKR antibodies in the human renal cell cancer xenograft 786-O after treatment with 0.3, 1.0 and 3.0 mg/kg (i.v., q4dx3) started at day 7 after tumor cell inoculation. Shown are final tumor weights at day 40. A is “Vehicle group, treated with PBS (i.v. q4dx3)”. B is “Isotype, 3 mg/kg”, C is “TPP-2084, 0.3 mg/kg”, D is “TPP-2084, 1 mg/kg”, E is “TPP-2084, 3 mg/kg”, F is “TPP-2090, 0.3 mg/kg”, G is “TPP-2090, 1 mg/kg”, H is “TPP-2090, 3 mg/kg”. (Y is “Tumor weights means of n=8; SD [g]”).

FIG. 20: Efficacy of 3 mg/kg TPP-2090 (i.v., q4dx7) in the human colon cancer xenograft WiDr in monotherapy and combination therapy with Irinotecan (5 mg/kg, i.v., 4d on, 3d off) and Regorafenib (10 mg/kg, p.o., daily). Treatment started 7d after inoculation with established tumors of about 40 mm². A is “Vehicle group, treated with PBS (i.v. q4dx7)”. B is “TPP-2090, 3 mg/kg”, C is “TPP-2090, 10 mg/kg”, D is “Irinotecan, 5 mg/kg”, E is “Combo TPP-2090 3 mg/kg+Irinotecan, 5 mg/kg”, F is “Regorafenib, 10 mg/kg”, G is “Combo TPP-2090, 3 mg/kg+Regorafenib 10 mg/kg”. (X is “Time after inoculation [days]”, Y is “Tumor area, means of n=10; SD [mm²])

FIG. 21: Efficacy of 10 mg/kg TPP-2090 (i.v., q4dx8) in the human lung cancer xenograft NCI-H322 in monotherapy and combination therapy with Paclitaxel (16 mg/kg, i.v., q7dx4). Treatment started 14d after inoculation with established tumors of about 45 mm². A is “Vehicle group, treated with PBS (i.v. q4dx8)”. B is “TPP-2090, 5 mg/kg”, C is “TPP-2090, 10 mg/kg”, D is “Paclitaxel, 16 mg/kg”, E is “Combo TPP-2090 10 mg/kg+Paclitaxel 16 mg/kg”. (X is “Time after inoculation [days]”; Y is “Tumor area, means of n=10; SD [mm²]”)

FIG. 22: Reduction of proliferative cells in xenografts after treatment with antibodies of the invention. Cryo sections from WiDr xenograft tumors after treatment with PBS (i.v., q4dx7: A) or TPP-2090 (10 mg/kg, i.v. q4dx7:B) were stained for the proliferation marker Ki67 by immunohistochemistry. Treatment started at day 7 after tumor cell inoculation and cryo sections were prepared from tumors taken at the end of the study (day 29). N=3 tumors per group were analyzed and representative images are shown. Treatment with TPP-2090 leads to a strong reduction of Ki67 positive cells (cells with dark staining in image) in WiDr xenograft tumors in mice.

FIG. 23: Induction of Stat-1 and NF-kappaB2 signaling pathways by anti-TWEAKR antibodies in vivo. Lysates of snap frozen WiDr xenograft tumors after treatment with PBS (i.v., q4dx7: lanes 1&2) or TPP-2090 (3 mg/kg, i.v., q4dx7: lanes 3&4) were subjected to Western Blot analysis detected with specific antibodies for P-Stat 1 (a), Stat-1 (b), NF-kappa2-p52 (c) and GAPDH (d). Treatment of mice started at day 7 after tumor cell inoculation and lysates were prepared from snap frozen tumors taken at the end of the study (day 29). Blots from 2 representative animals per group are shown. Treatment with TPP-2090 leads to a strong induction of P-Stat 1 & Total Stat 1 levels as well as NF-kappaB2 activation (shown by the appearance of the p52 band) in WiDr xenograft tumors.

FIG. 24: Consensus sequences for anti-TWEAKR antibodies. CDR-H1—X at position 5: M or I; CDR-H2—X at position 8: S or K; CDR-L1—X at position 8: G or S; CDR-L2—X at position 1: N, A or Q; CDR-L3—X at position 5: T or S; X at position 6: S or T; X at position 8: F or G

FIG. 25: Continuous CDR sequence nomenclature. (A) Positions in boxes were diversified for mutation gathering (maturation process). (B) Single substitutions in boxes were recombined in one recombination library.

FIG. 26: Sequences of the invention

### DETAILED DESCRIPTION OF THE INVENTION

The present invention is based on the discovery of novel antibodies that have a specific affinity for TWEAKR and can deliver a therapeutic benefit to a subject. The antibodies of the invention, which may be human, humanized or chimeric, can be used in many contexts, which are more fully described herein.

### DEFINITIONS

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent with the meaning commonly understood in the art. Such references include, but are not limited to, Singleton et al, Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company. Any additional technical resource available to the person of ordinary skill in the art providing definitions of terms used herein having the meaning commonly understood in the art can be consulted. For the purposes of the present invention, the following terms are further defined. Additional terms are defined elsewhere in the description. As used herein and in the appended claims, the singular forms “a,” and “the” include plural reference unless the context clearly dictates otherwise. Thus, for example, reference to “a gene” is a reference to one or more genes and includes equivalents thereof known to those skilled in the art, and so forth.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Soderlind et al., Nature Biotech. 2000, 18:853-856.

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non-human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies. An “agonist/agonistic antibody” as used herein is an antibody which mimics at least one of the functional activities of a polypeptide of interest (here the TWEAKR ligand TWEAK).

As used herein, an antibody “binds specifically to”, is “specific to/for” or “specifically recognizes” an antigen of interest, e.g. a tumor-associated polypeptide antigen target (here, TWEAKR), is one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using a Biacore T100 instrument (GE Healthcare Biacore, Inc.) according to Example 2. Other suitable devices are BIACORE T200, BIACORE(R)-2000, BIACORe 4000, a BIACORE (R)-3000 (BIAcore, Inc., Piscataway, N.J.), or ProteOn XPR36 instrument (Bio-Rad Laboratories, Inc.).

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs. arranged from amino terminus to carboxy-terminus e.g. in the following order: FR1, CDR1, FR2, CDR2, FR3, CDR3, FR4.

As used herein, the term “Complementarity Determining Regions (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA, and IgA2. The heavy-chain constant domains that correspond to the different classes of antibodies are called [alpha], [delta], [epsilon], [gamma], and [mu], respectively. The subunit structures and three-dimensional configurations of different classes of immunglobulins are well known. As used herein antibodies are conventionally known antibodies and functional fragments thereof.

A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or −3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG.

“Functional fragments” or “antigen-binding antibody fragments” of the invention include Fab, Fab′, F(ab′)2, and Fv fragments; diabodies; single domain antibodies (DAbs), linear antibodies; single-chain antibody molecules (scFv); and multispecific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “multi-specific” or “multi-functional” antibody is understood to have each of its binding sites identical. The F(ab′)2 or Fab may be engineered to minimize or completely remove the intermolecular disulphide interactions that occur between the CH1 and CL domains.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991

Variants of the antibodies or antigen-binding antibody fragments contemplated in the invention are molecules in which the binding activity of the antibody or antigen-binding antibody fragment for TWEAKR is maintained.

Binding proteins contemplated in the invention are for example antibody mimetics, such as Affibodies, Adnectins, Anticalins, DARPins, Avimers, Nanobodies (reviewed by Gebauer M. et al., Curr. Opinion in Chem. Biol. 2009; 13:245-255; Nuttall S. D. et al., Curr. Opinion in Pharmacology 2008; 8:608-617).

As used herein, the term “epitope” includes any protein determinant capable of specific binding to an immunoglobulin or T-cell receptors. Epitopic determinants usually consist of chemically active surface groupings of molecules such as amino acids or sugar side chains, or combinations thereof and usually have specific three dimensional structural characteristics, as well as specific charge characteristics.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes. In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or nonreducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

“Antibody-dependent cell-mediated cytotoxicity” or “ADCC” refers to a form of cytotoxicity in which secreted Ig bound onto Fc gamma receptors (FcγRs) present on certain cytotoxic cells (e.g. NK cells, neutrophils, and macrophages) enable these cytotoxic effector cells to bind specifically to an antigen-bearing target cell and subsequently kill the target cell e.g. with cytotoxins. To assess ADCC activity of an antibody of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells.

“Complement dependent cytotoxicity” or “CDC” refers to the lysis of a target cell in the presence of complement. Activation of the classical complement pathway is initiated by the binding of the first component of the complement system (C1 q) to antibodies (of the appropriate subclass), which are bound to their cognate antigen. To assess complement activation, a CDC assay, e.g., as described in Gazzano-Santoro et al., J. Immunol. Methods 202: 163 (1996), may be performed. Polypeptide variants with altered Fc region amino acid sequences (polypeptides with a variant Fc region) and increased or decreased C1q binding are described, e.g., in U.S. Pat. No. 6,194,551 B1 and WO 1999/51642.

The term immunoconjugate (interchangeably referred to as “antibody-drug conjugate,” or “ADC”) refers to an antibody conjugated to one or more cytotoxic or cytostatic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate). Immunoconjugates have been used for the local delivery of cytotoxic agents, i.e., drugs that kill or inhibit the growth or proliferation of cells, in the treatment of cancer (e.g. Liu et al., Proc Natl. Acad. Sci. (1996), 93, 8618-8623)). Immunoconjugates allow for the targeted delivery of a drug moiety to a tumor, and intracellular accumulation therein, where systemic administration of unconjugated drugs may result in unacceptable levels of toxicity to normal cells and/or tissues. Toxins used in antibody-toxin conjugates include bacterial toxins such as diphtheria toxin, plant toxins such as ricin, small molecule toxins such as geldanamycin. The toxins may exert their cytotoxic effects by mechanisms including tubulin binding, DNA binding, or topoisomerase inhibition.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term ‘maturated antibodies’ or ‘maturated antigen-binding fragments’ such as maturated Fab variants includes derivatives of an antibody or antibody fragment exhibiting stronger binding—i. e. binding with increased affinity—to a given antigen such as the extracellular domain of the TWEAKR. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

An “agonistic” antibody or an antibody with “agonistic activity” is one that binds to its target and induces the activation of the respective target, that e.g. leads to activation of the signaling pathways or biological effects that are mediated by the respective target.

### Antibodies of the Invention

The invention is related to antibodies, or antigen-binding antibody fragments thereof, or variants thereof which lead to strong activation of the TWEAKR (SEQ ID NO:169 (protein); SEQ ID NO:170 (DNA)), thus leading to a strong induction of apoptosis in various cancer cells showing overexpression of the TWEAKR.

TWEAKR agonistic activity with regard to induction of apoptosis and inhibition of proliferation of the anti-TWEAKR antibodies described previously (e.g. PDL-192) is limited and does not reach the efficacy of the endogenous ligand TWEAK. This lack of agonistic activity is not due to a decreased affinity as these antibodies bind to the TWEAKR with affinities in a similar range as compared to the endogenous ligand TWEAK (Michaelson J S et al, MAbs. 2011 Jul.-Aug.; 3(4):362-75; Culp P A et al, Clin Cancer Res. 2010 Jan. 15; 16(2):497-508) and also antibodies with higher binding affinity do not necessarily exhibit more efficacious signaling activity (Culp P A, et al, Clin Cancer Res. 2010 Jan. 15; 16(2):497-508). In addition, anti-tumor activity of the antibodies described previously is shown to be dependent on Fc effector function and ADCC is shown to play a significant role for the in vivo efficacy in mouse models.

The invention provides antibodies, antigen-binding fragments thereof, or variants thereof, which have such a strong agonistic activity with regard to induction of apoptosis and inhibition of proliferation that in vivo anti-tumor efficacy can be achieved without ADCC playing a significant role. The skilled artesian knows methods to provide antibody variants lacking Fc gamma receptor activation to prevent ADCC while maintaining antigen binding and agonistic activity. Such methods include but are not limited to the use of human IgG2 and human IgG4 antibody isotypes, to the use of aglycosylated antibodies, or to the use of antibodies having mutations preventing Fc gamma receptor activation.

It is an embodiment of the invention to provide antibodies, or antigen-binding antibody fragments thereof, or variants thereof, which have a strong induction of Caspase-3/7 in one or more TWEAKR expressing cell lines. In a preferred embodiment the one or more TWEAKR expressing cell line is comprised in the group consisting of WiDr, A253, NCI-H322, HT-29 and 786-O cells. “Induction of Caspase 3/7” can be measured by common methods known in the art, including those described herein. In one embodiment, the “Induction of Caspase 3/7” according to this invention is measured by using activity determination with Caspase 3/7 Solution (Promega, #G8093) and reading of luminescence on a VICTOR V (Perkin Elmer). At the end of the incubation time Caspase 3/7 activity was determined and the fold induction of Caspase 3/7 was calculated as compared to untreated cells. An antibody is said to have “strong induction” of Caspase-3/7 if the fold of induction is greater than 1.2, preferably greater than 1.5, more preferably greater than 1.8, more preferably greater than 2.1, more preferably greater than 2.5. Provided are anti-TWEAKR antibodies which lead to a stronger induction of Caspase 3/7 in HT-29 cells as compared to the agonistic antibodies previously described [e.g. PDL-192(TPP-1104), P4A8(TPP-1324), 136.1(TPP-2194)] and also as compared to 300 ng/ml recombinant human TWEAK. This strong efficacy to induce Caspase 3/7 in cancer cells was also seen in WiDr, A253, NCI-H322 and 786-O cells, where the tested antibodies of the invention induced higher fold-changes as compared to the reference antibodies [PDL-192(TPP-1104), P4A8(TPP-1324)] and 300 ng/ml TWEAK in most experiments. Some antibodies of the invention bind to the TWEAKR with only moderate affinity (>10 nM) that is clearly lower compared to the affinity of the endogenous ligand TWEAK and lower compared to other known agonistic antibodies. This property provides further potential advantages as e.g. potentially improved tumor penetration.

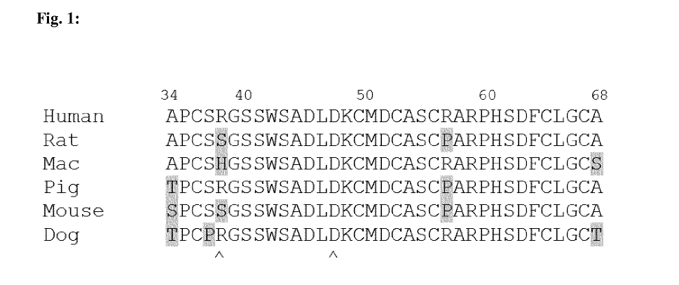

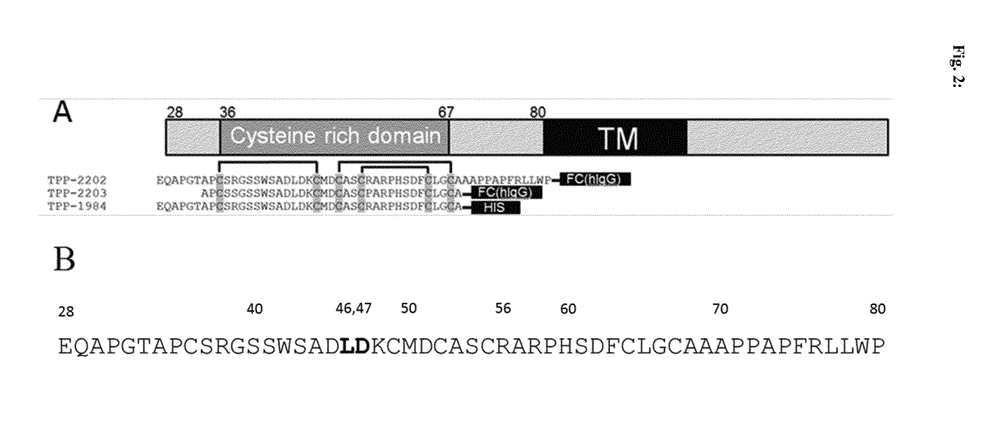

Toward these ends, it is an embodiment of the invention to provide antibodies, or antigen binding antibody fragments thereof, that specifically bind to a TWEAKR at a novel epitope characterized by selective binding to aspartate (D) at position 47 (D47) of TWEAKR (SEQ ID NO:169; and see FIG. 1). The identified dependencies on certain TWEAKR amino acids for antibody interaction correlate with the agonistic activity that has been determined for these antibodies. The native ligand TWEAK shows efficient activation of TWEAKR and binds dependent on Leucin 46 in the cysteine rich domain of TWEAKR (Pellegrini et al, FEBS 280:1818-1829). P4A8 shows very low agonistic activity and at least partially interacts with domains outside of the cysteine rich domain of TWEAKR. PDL-192 shows moderate agonistic activity and binds dependent of R56 to the cysteine rich domain but opposite to the TWEAK ligand site. Antibodies of this invention (exemplary TPP-2090) bind dependent on D47, and TWEAK binds dependent on L46, and binds to a similar but distinguishable binding site (FIG. 7). Therefore the antibodies of this invention which show a strong agonistic activity bind to a novel epitope (D47 dependent) for antibodies which is connected to very strong agonistic activity.

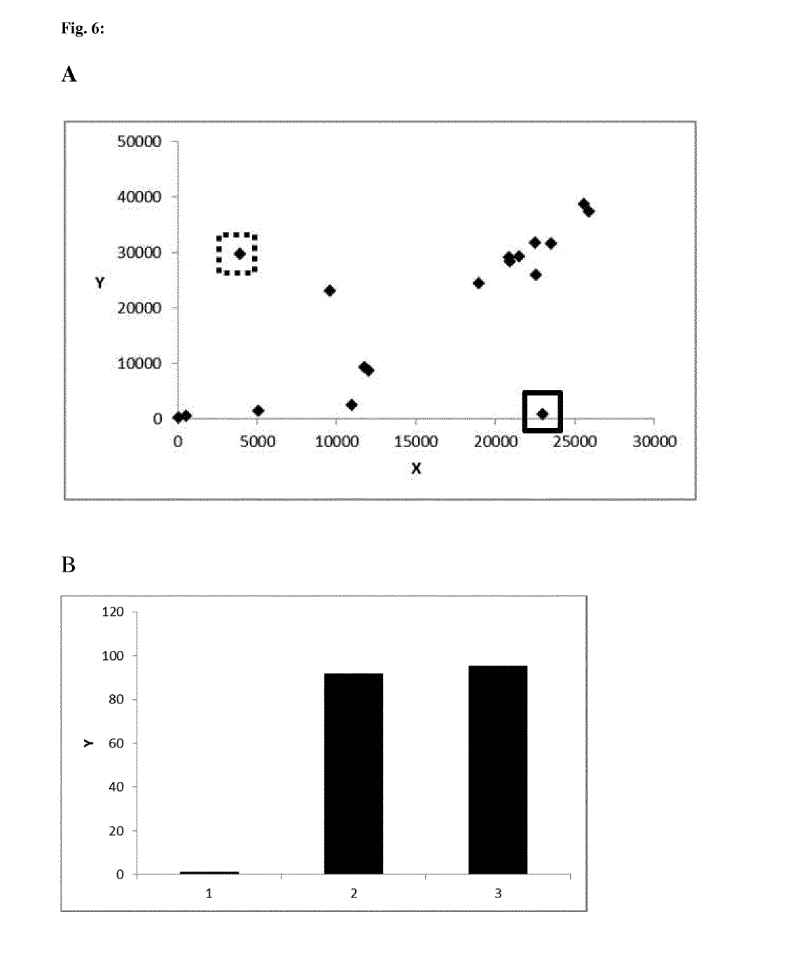

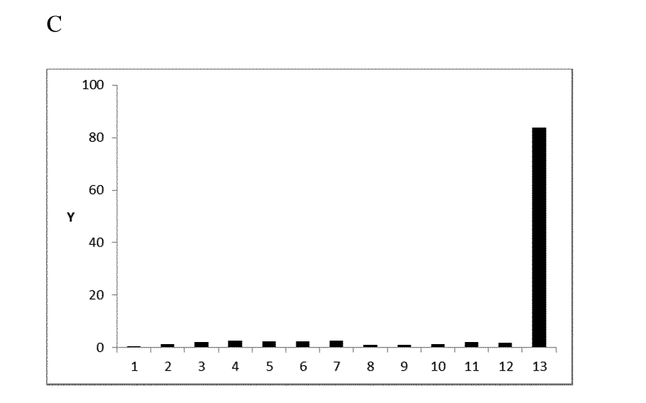

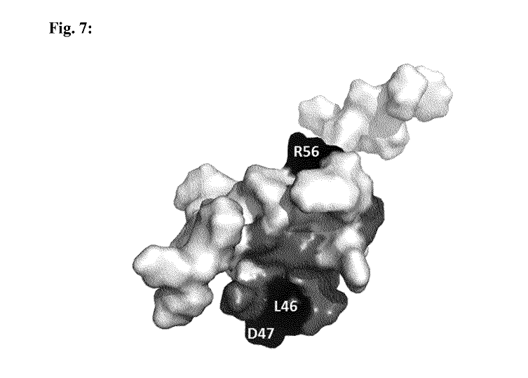

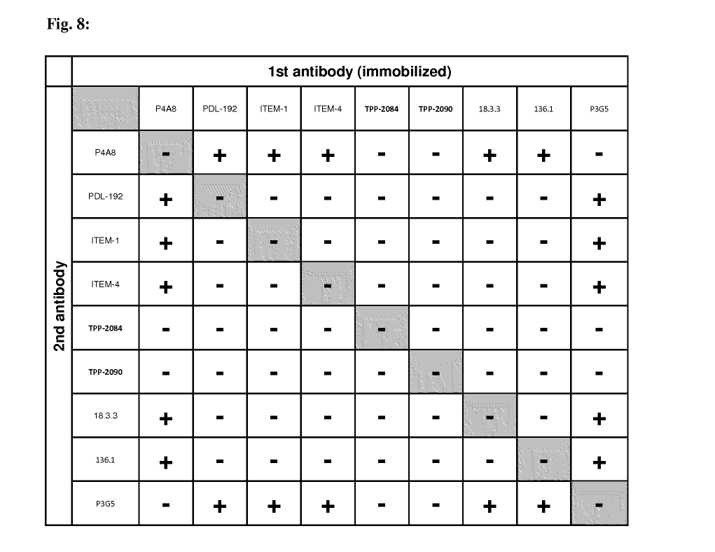

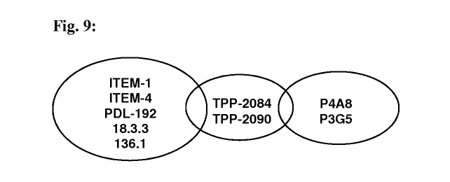

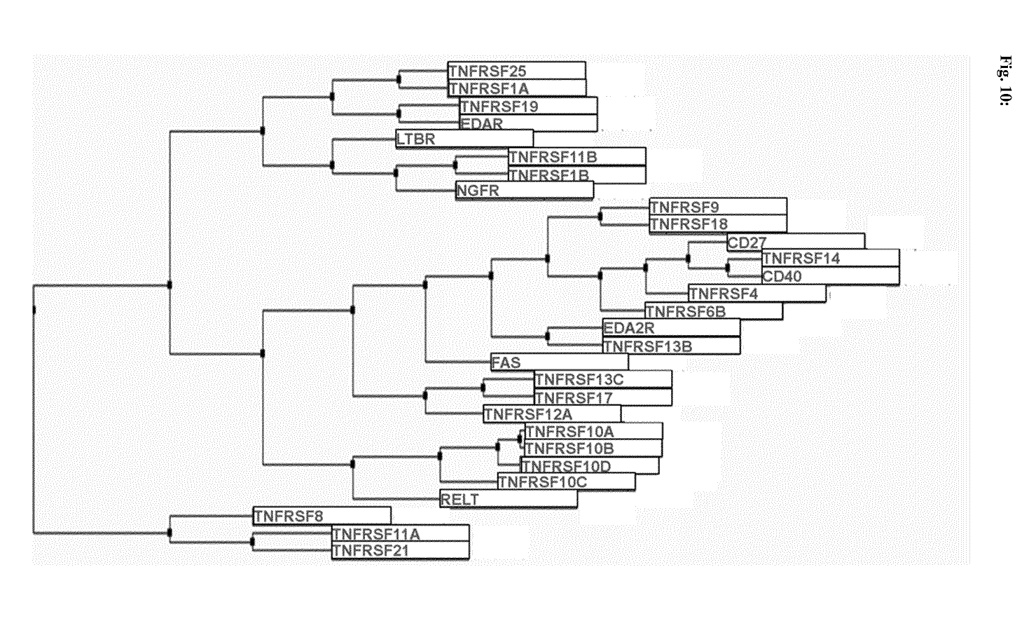

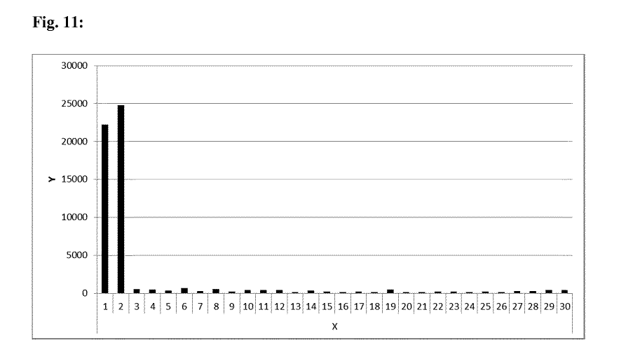

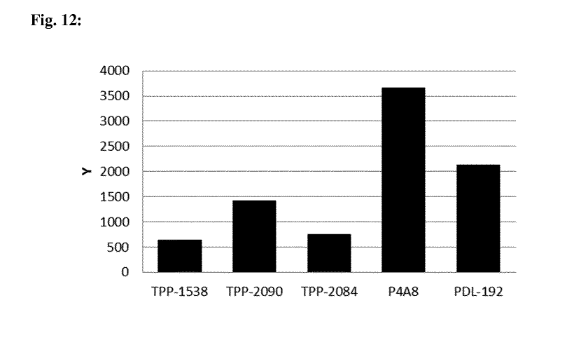

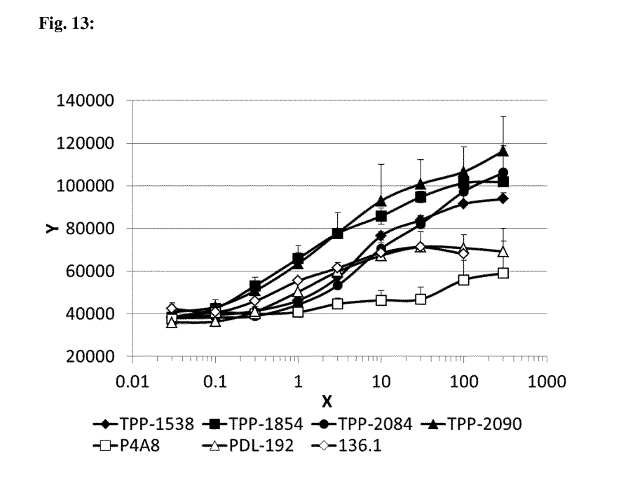

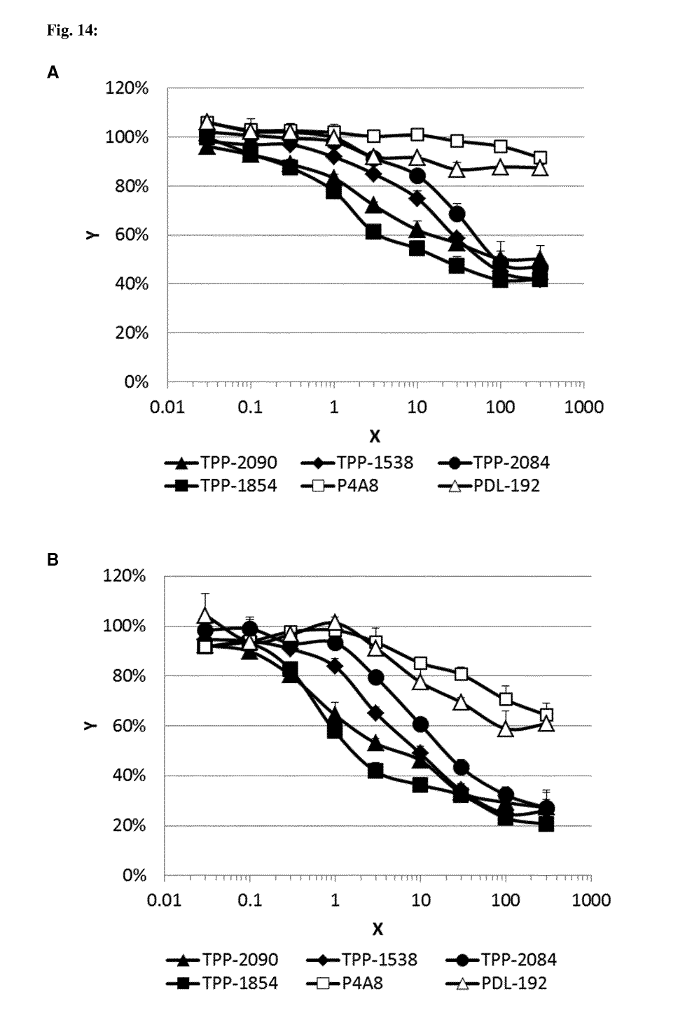

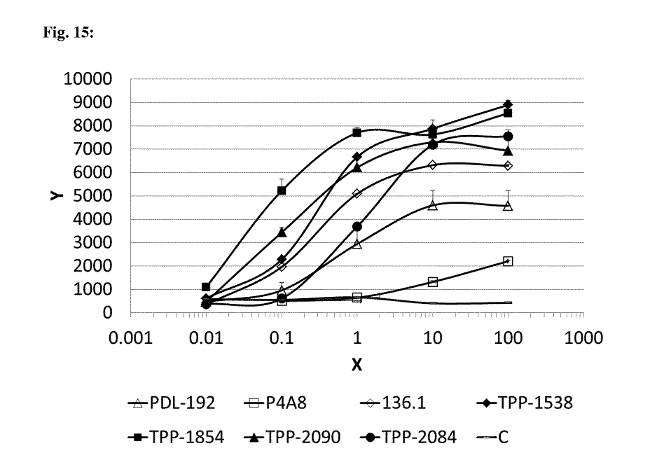

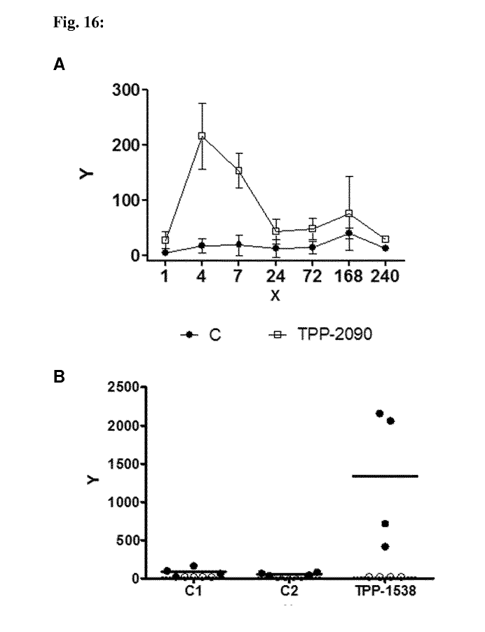

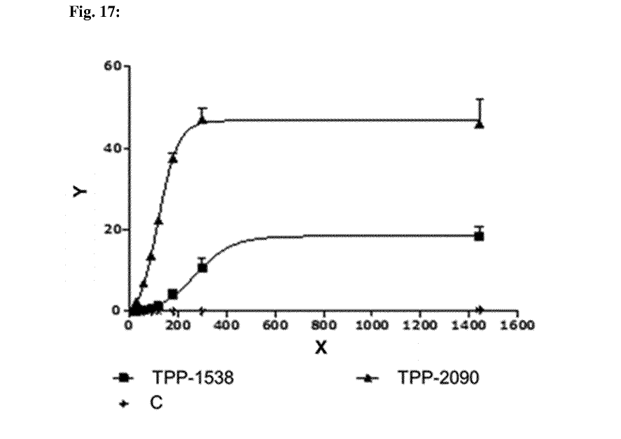

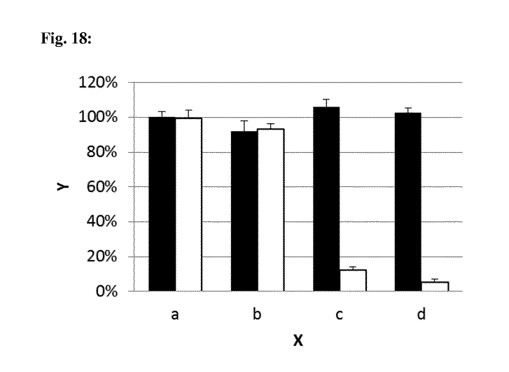

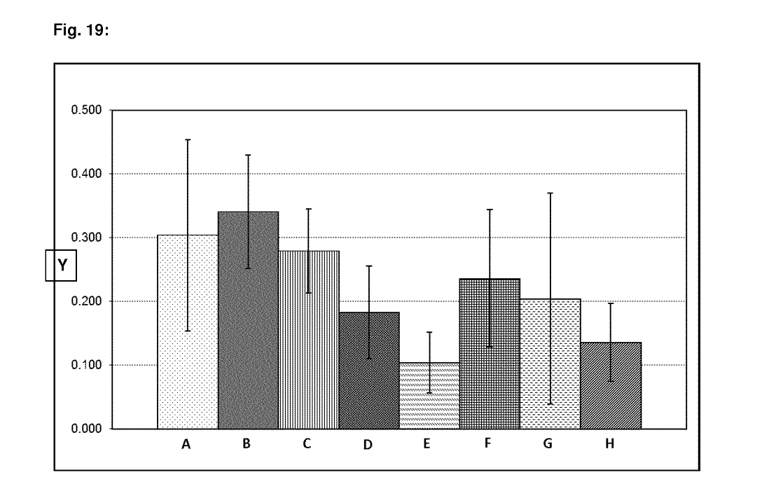

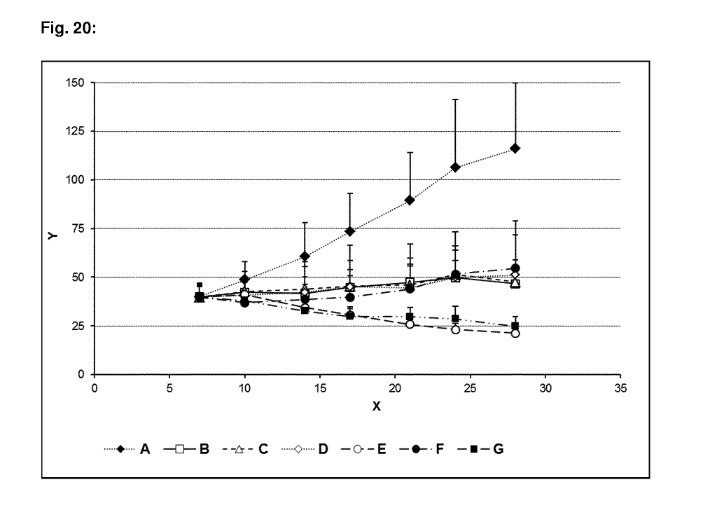

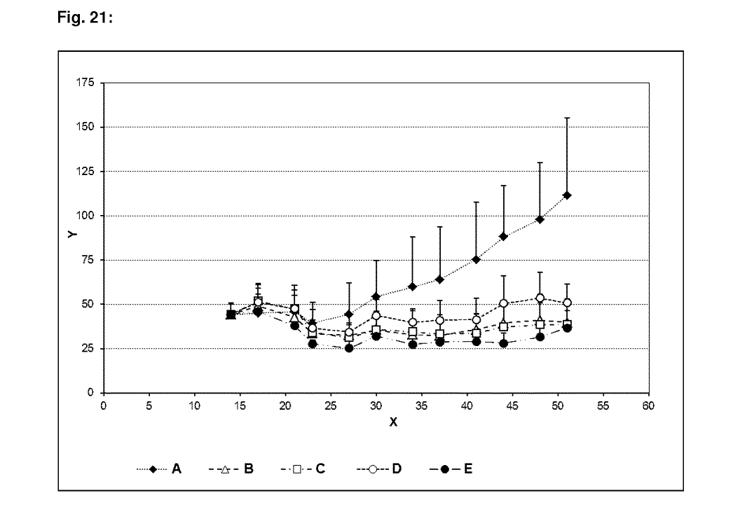

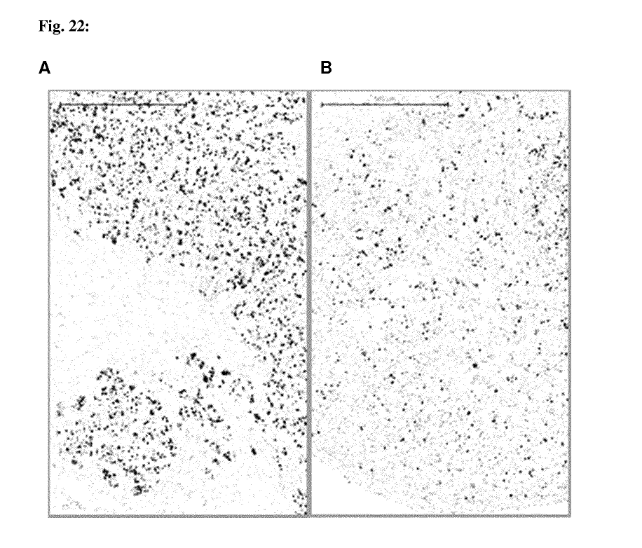

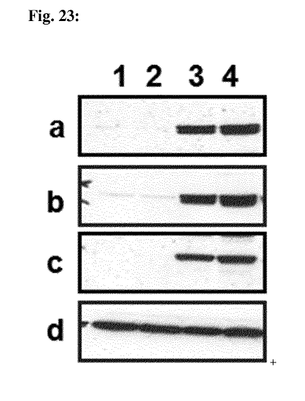

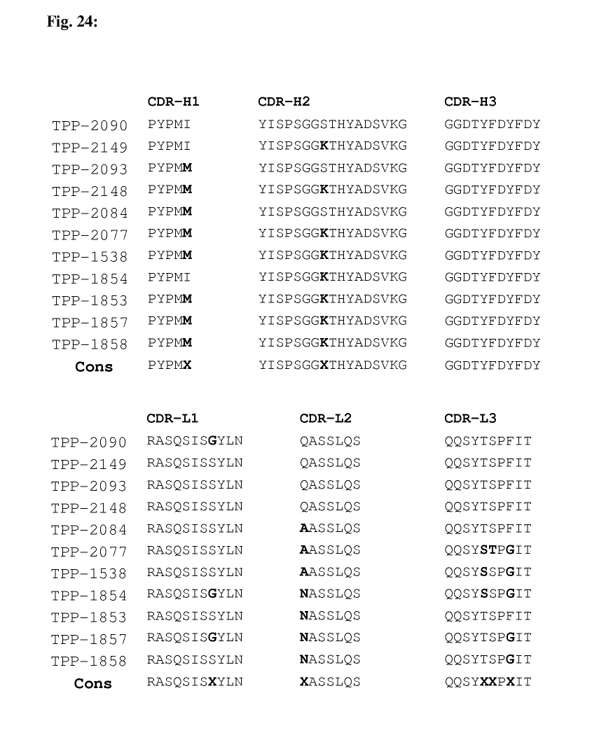

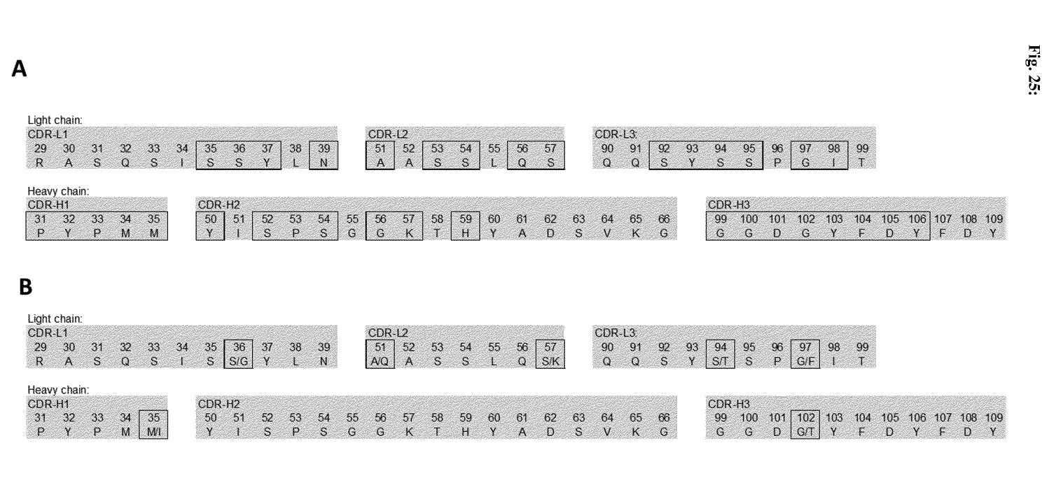

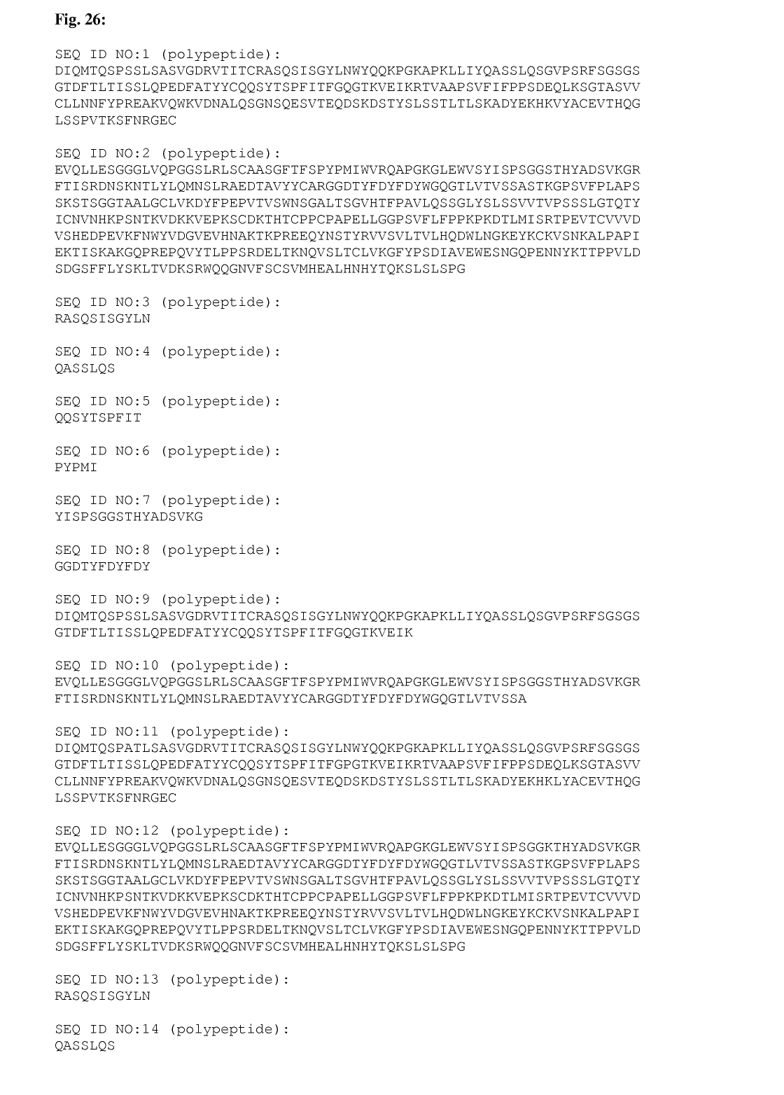

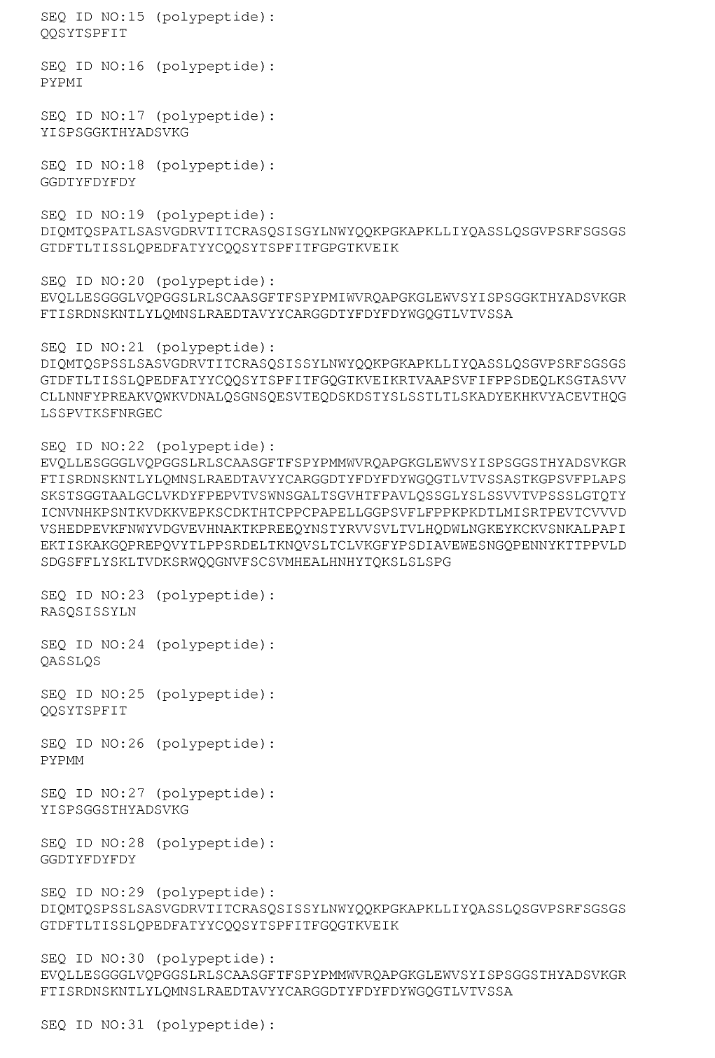

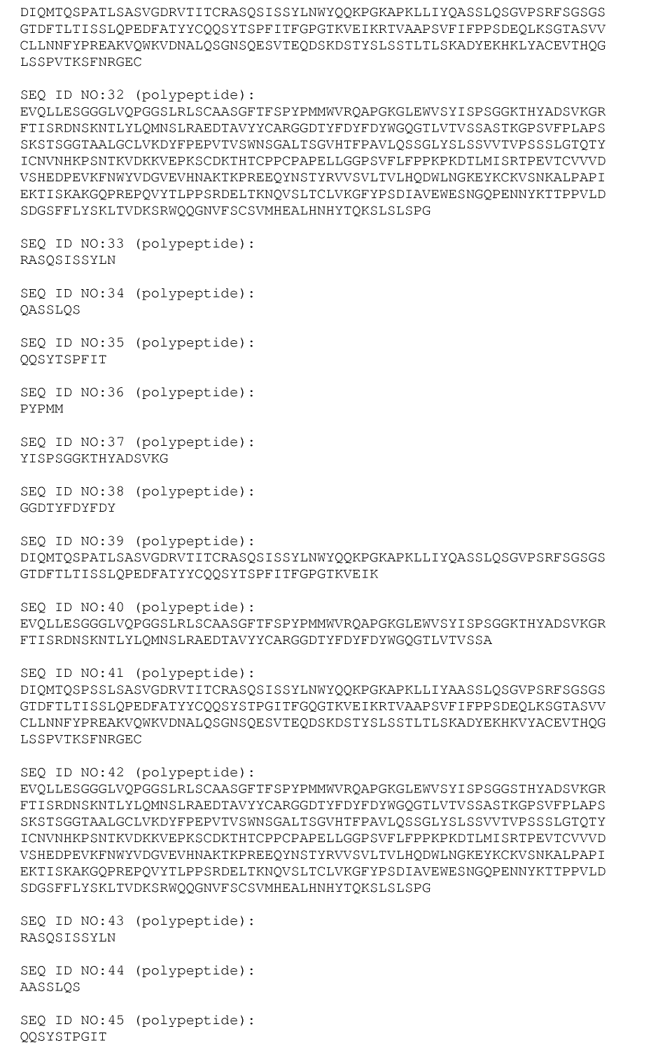

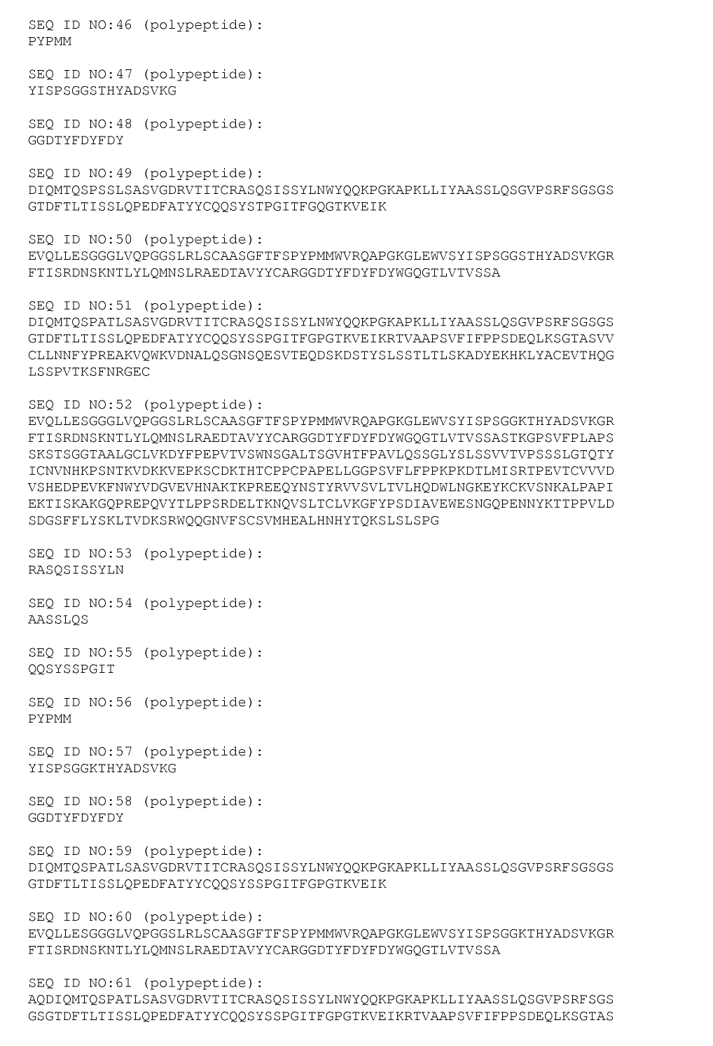

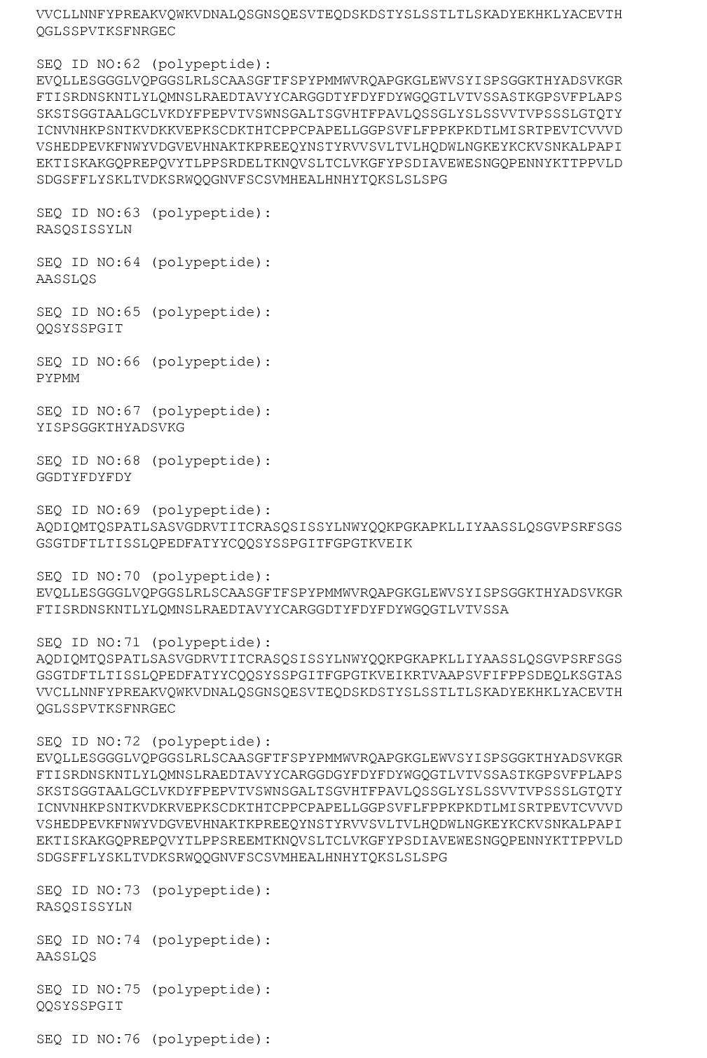

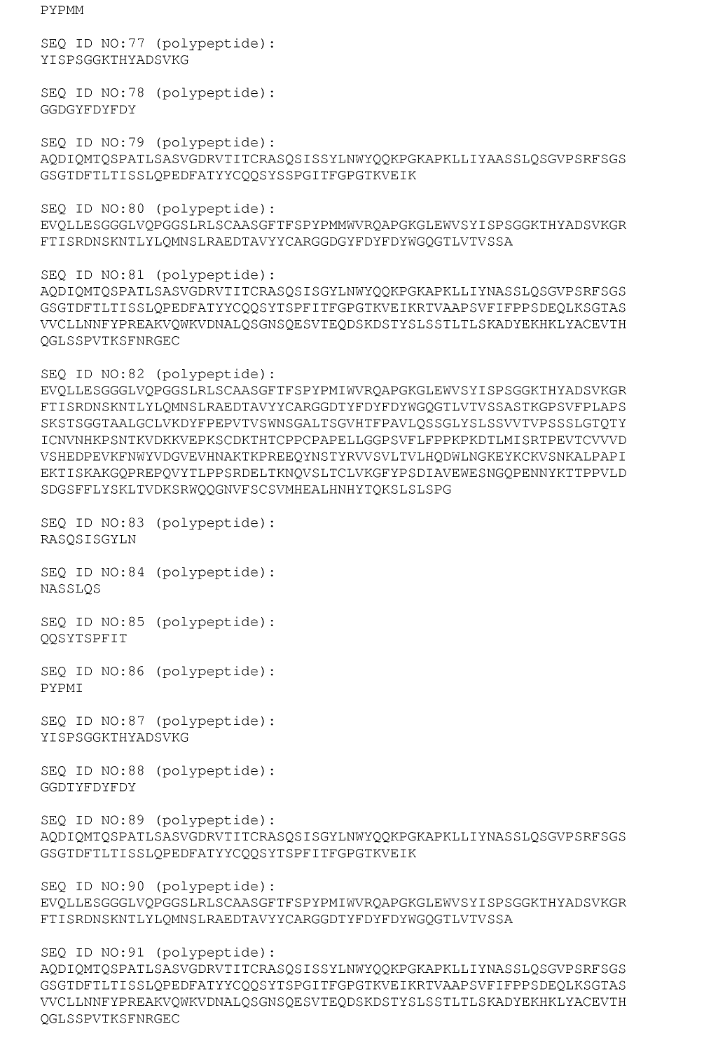

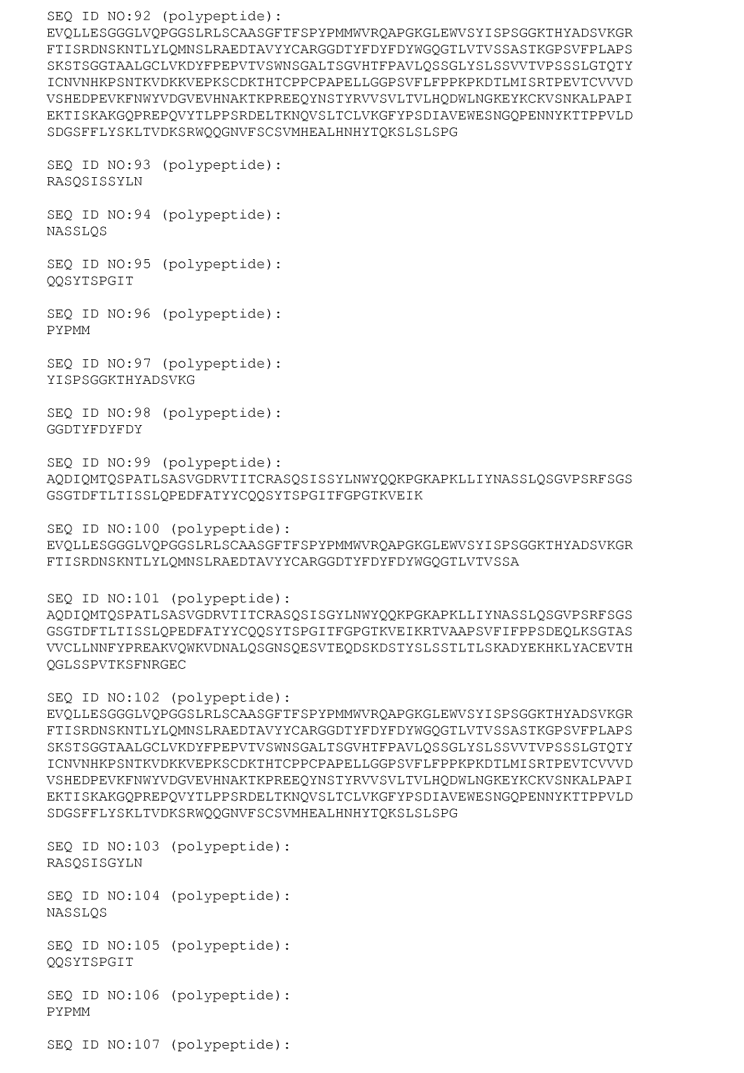

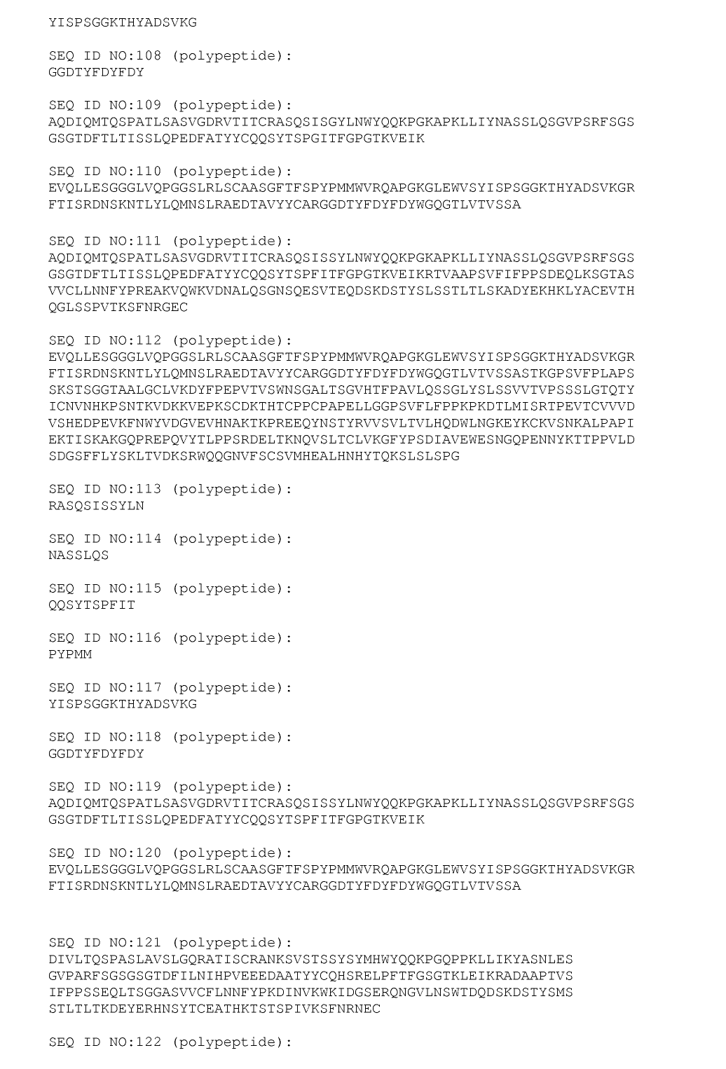

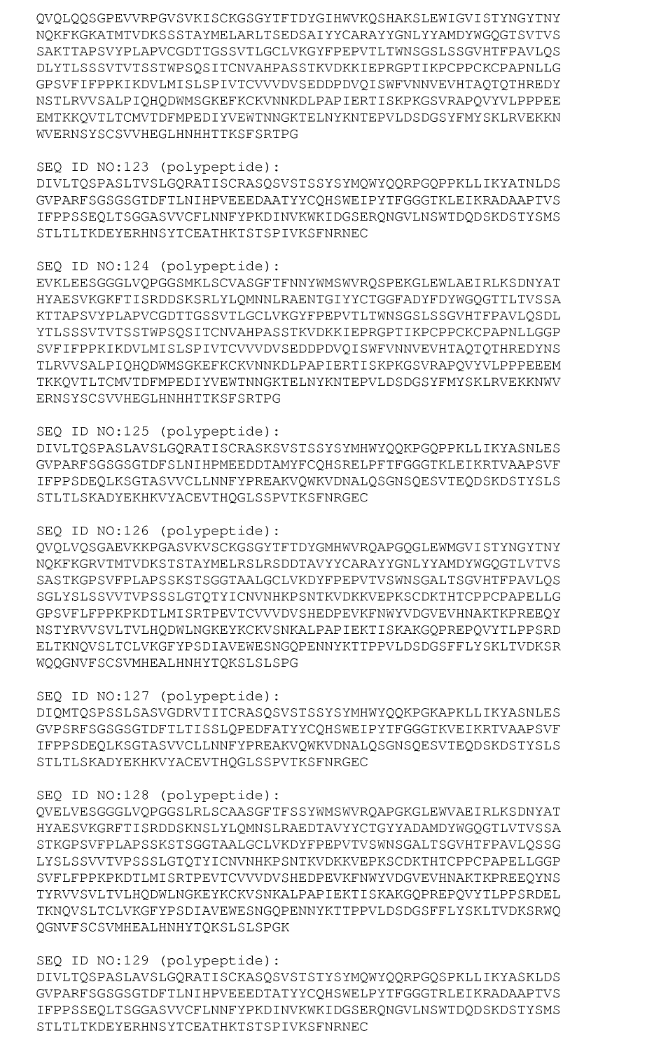

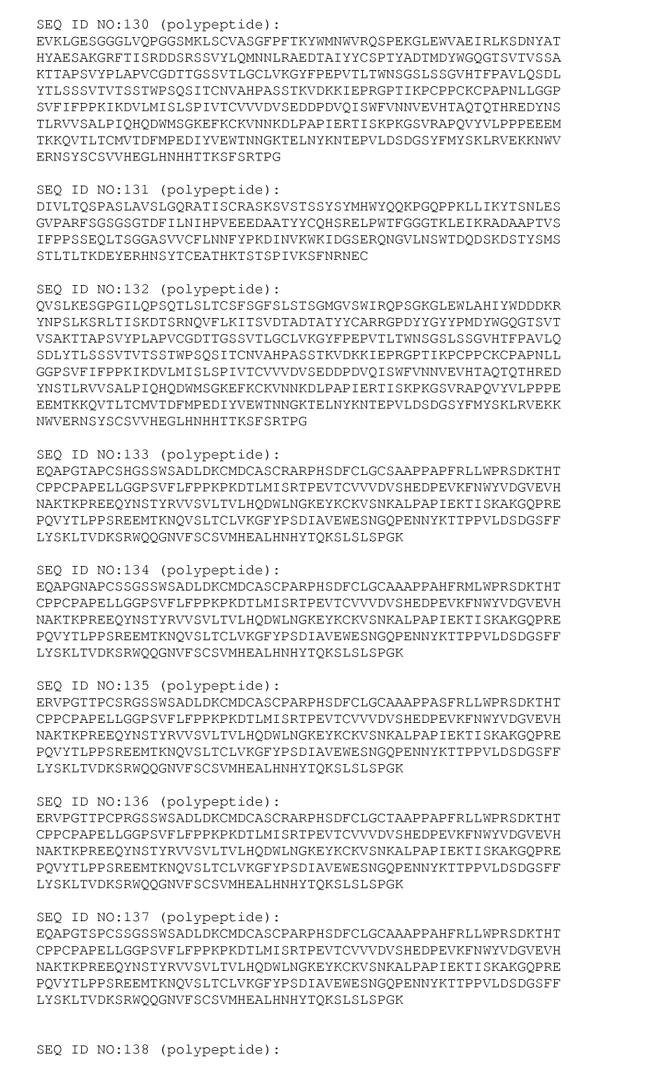

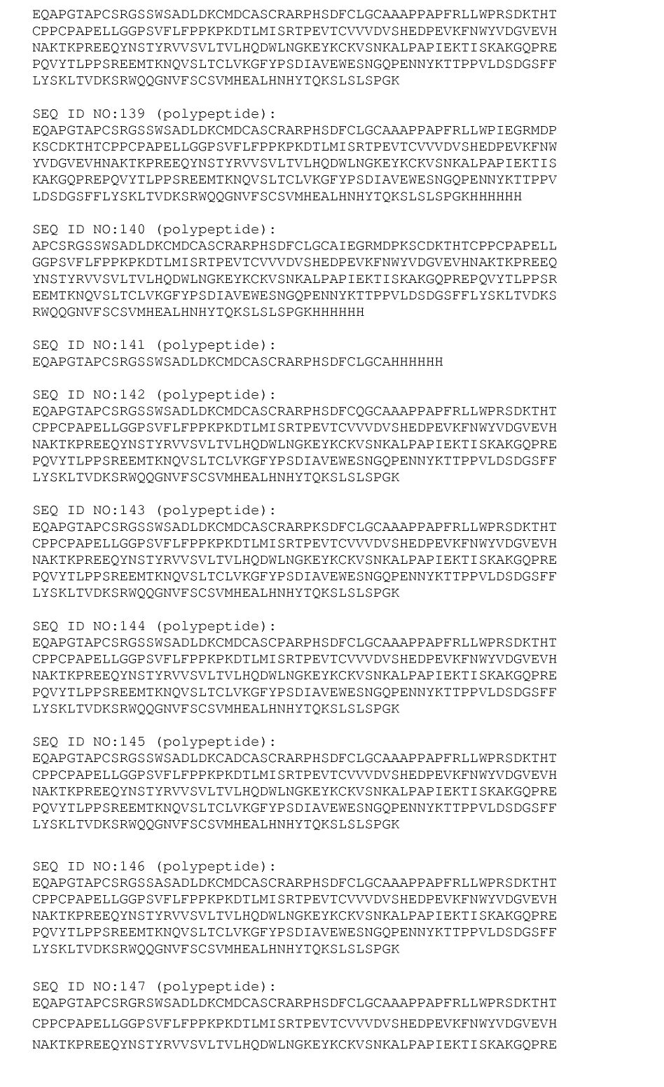

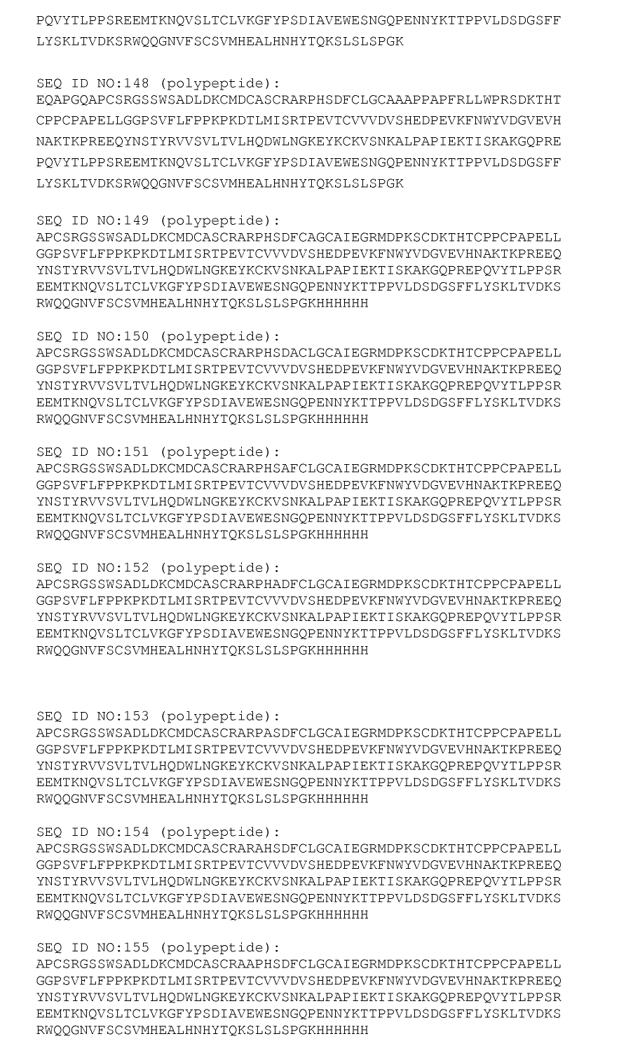

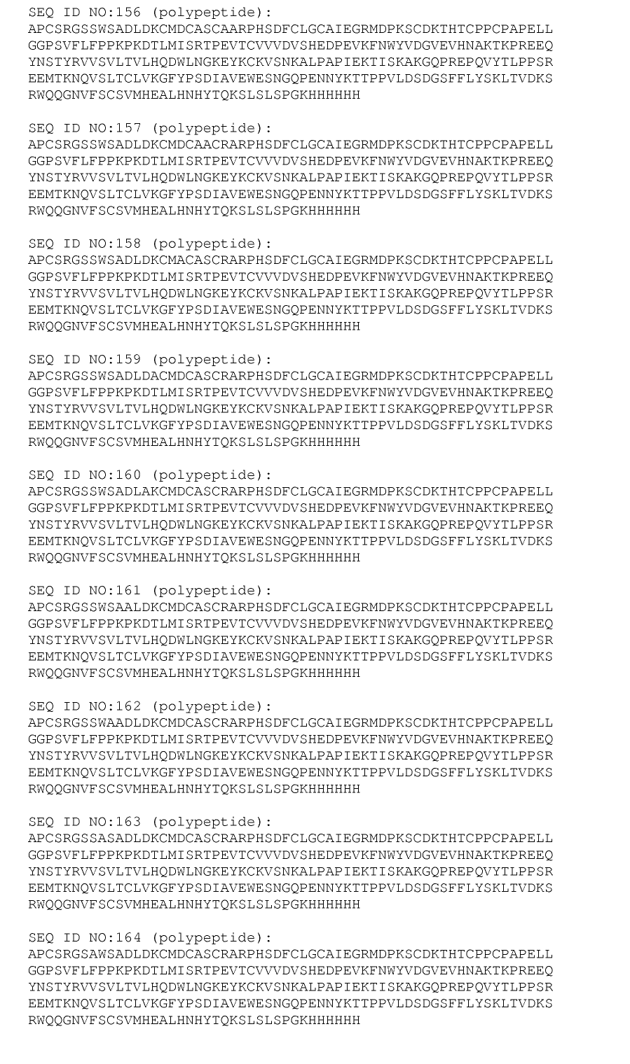

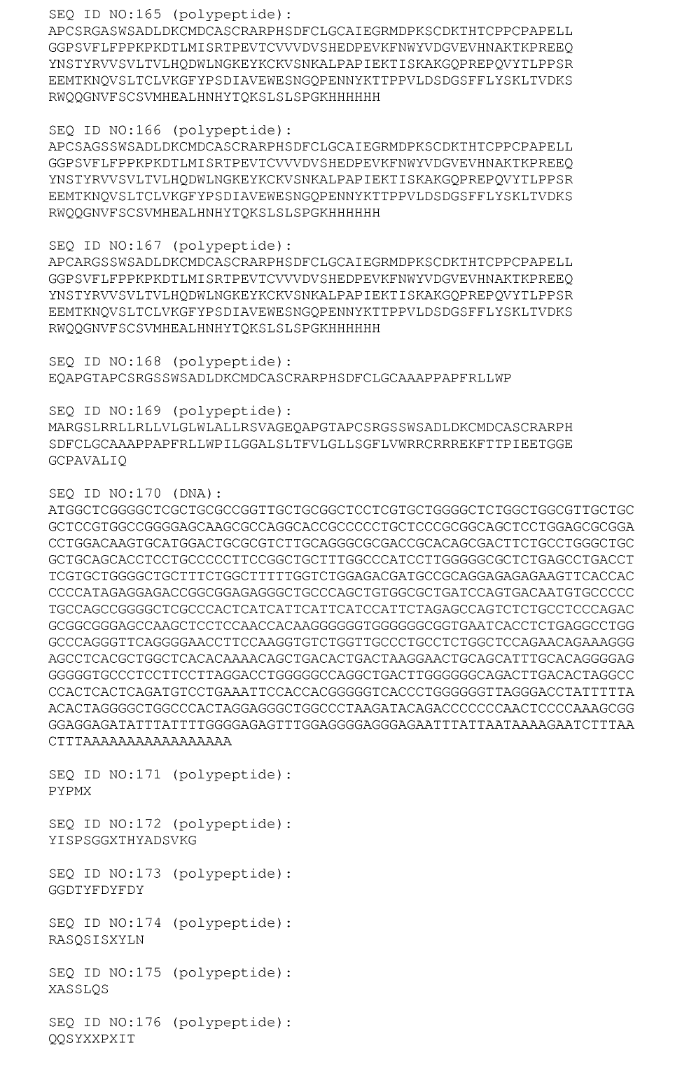

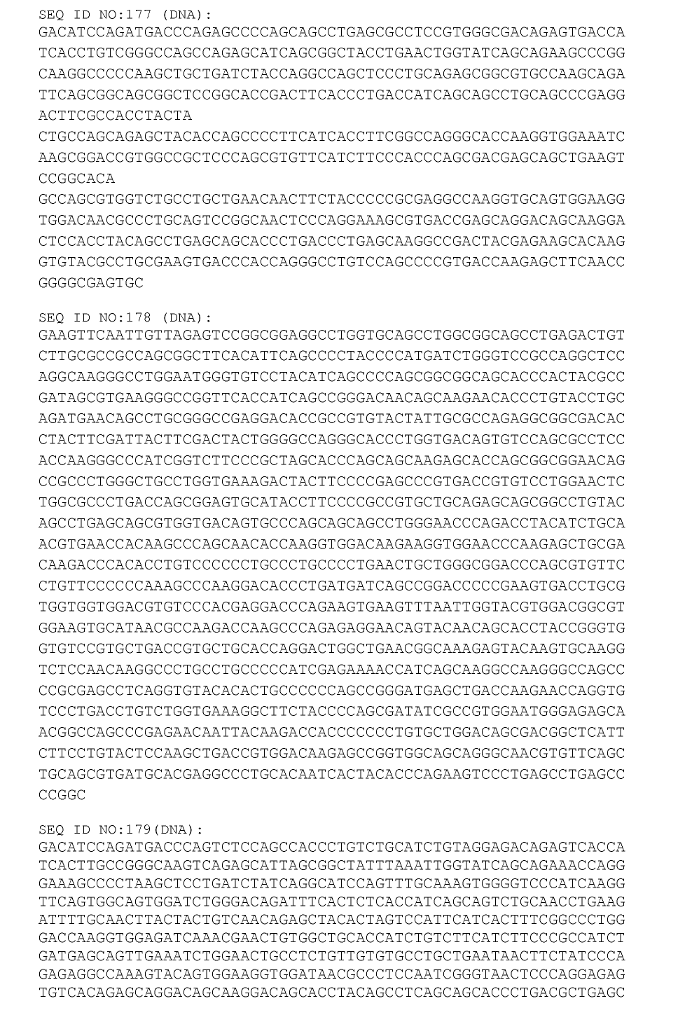

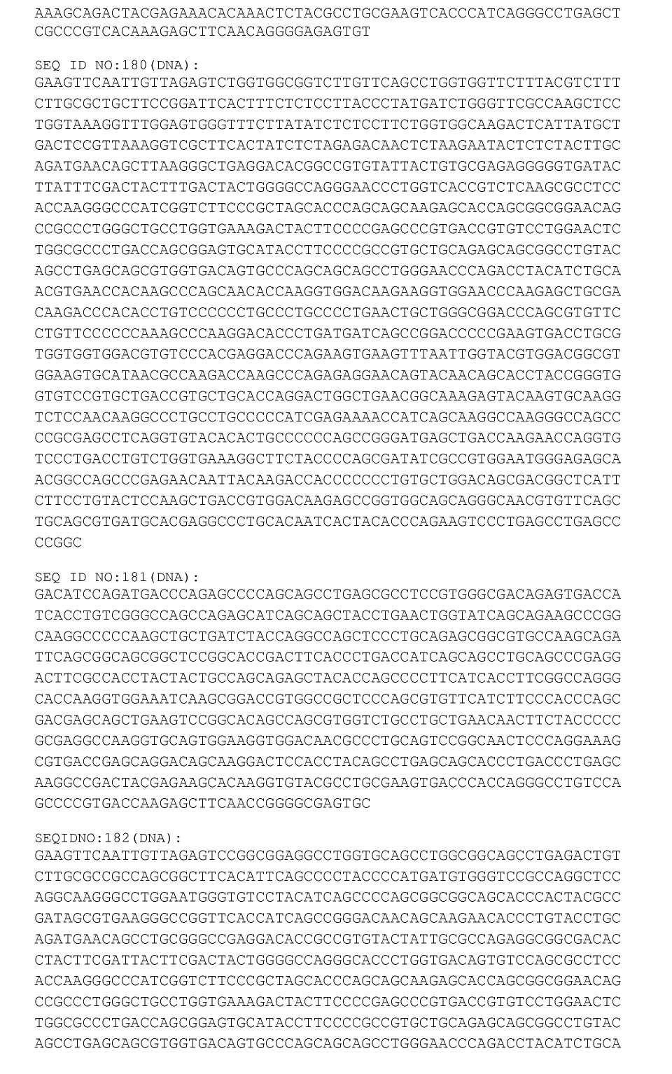

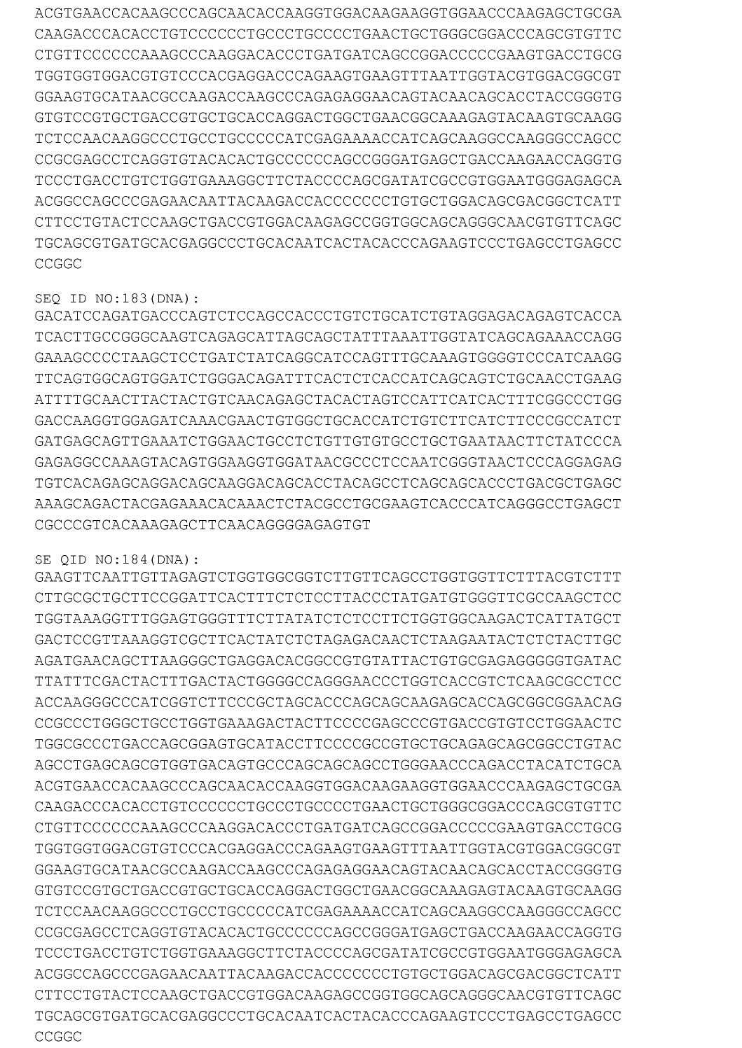

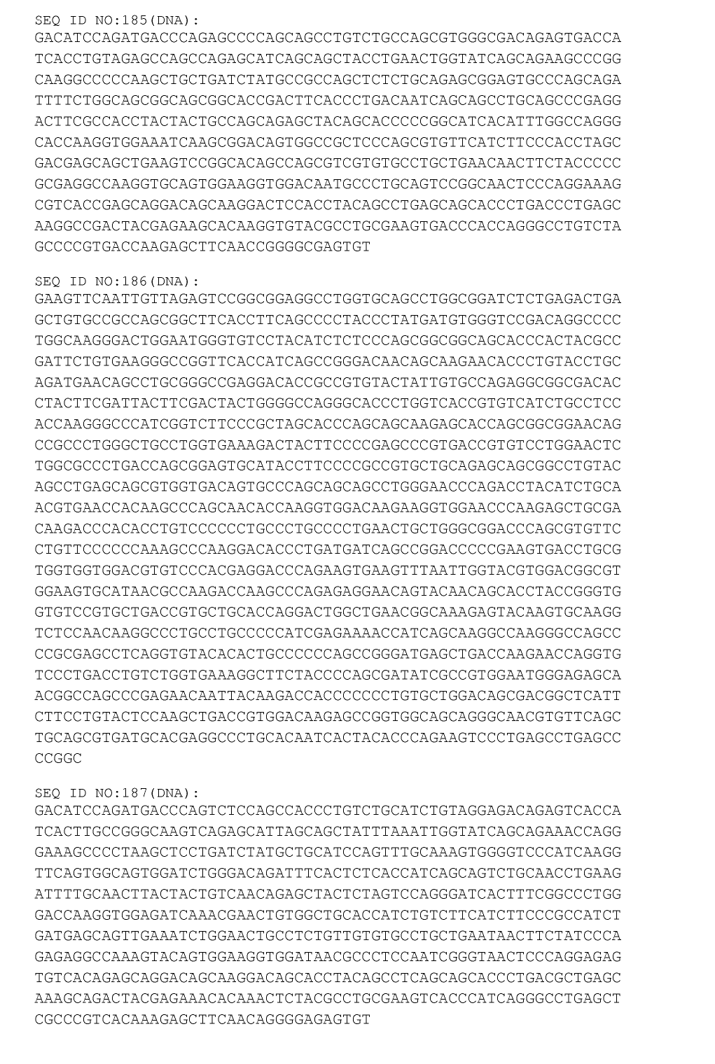

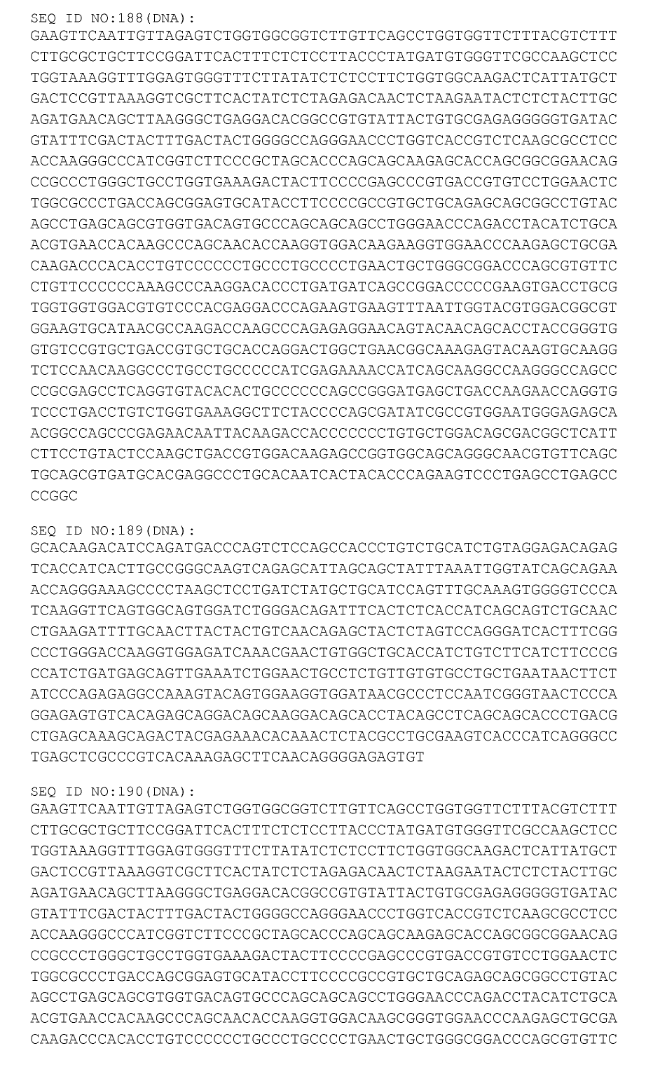

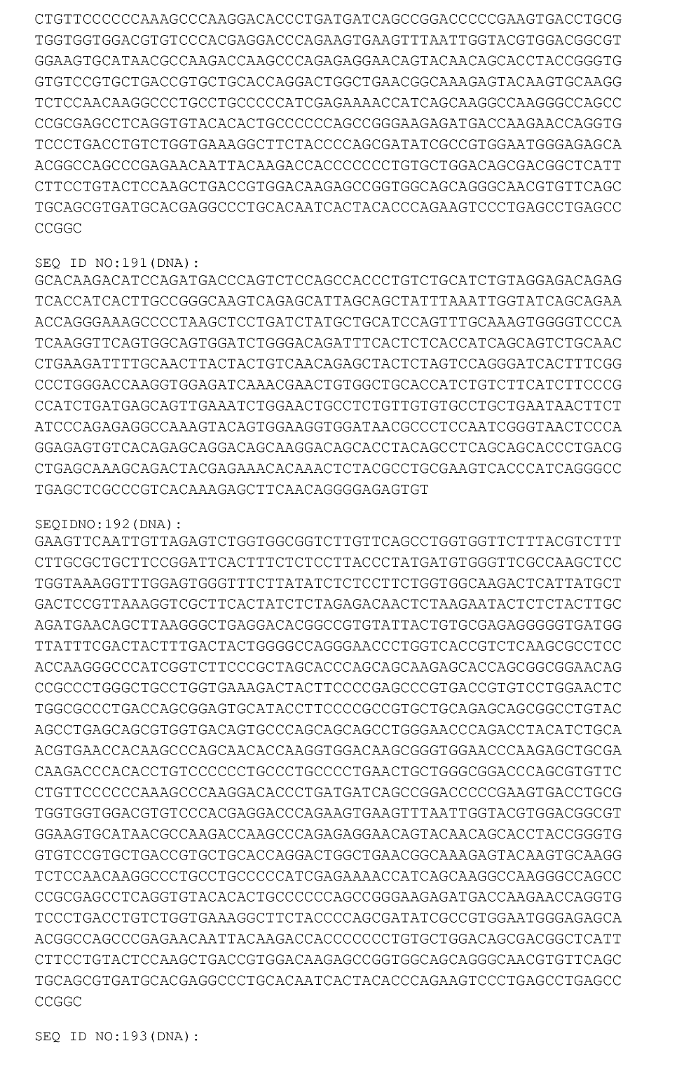

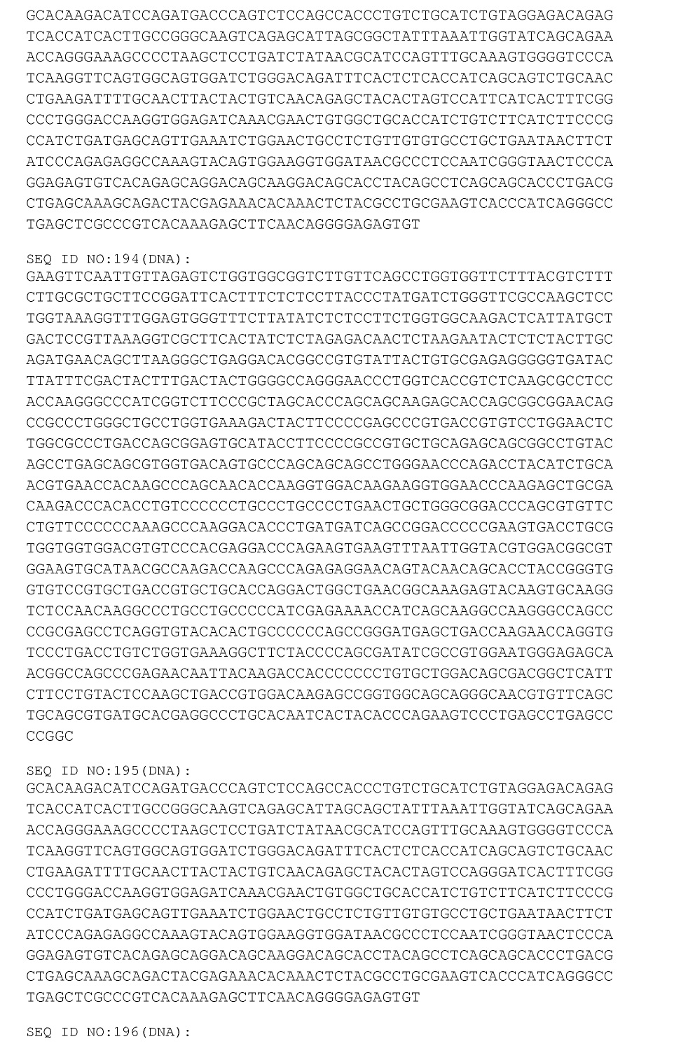

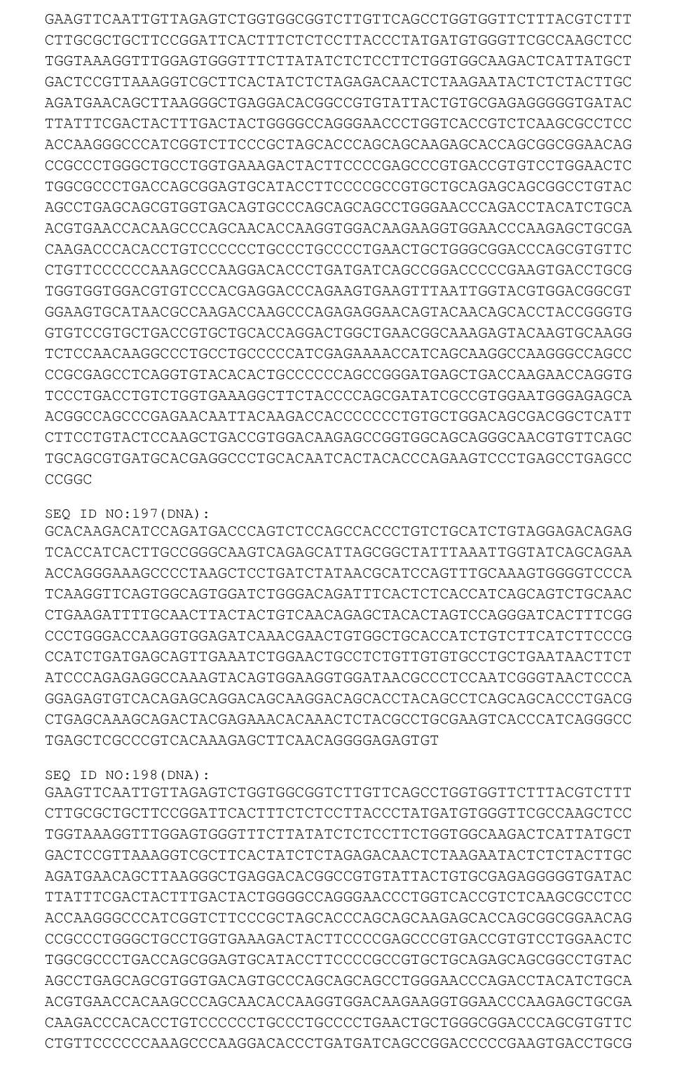

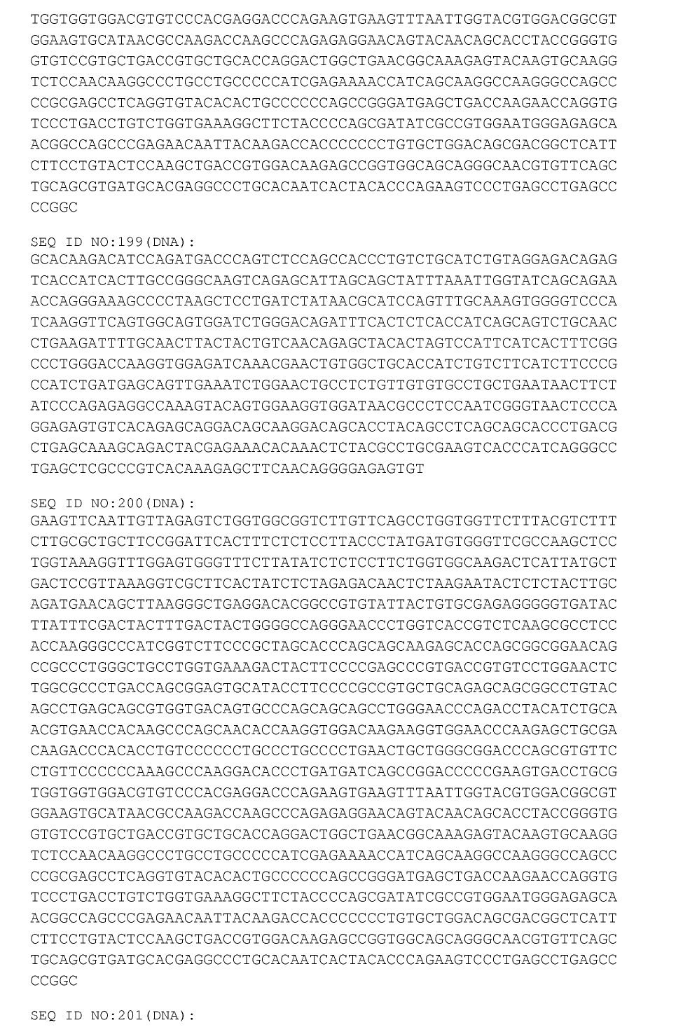

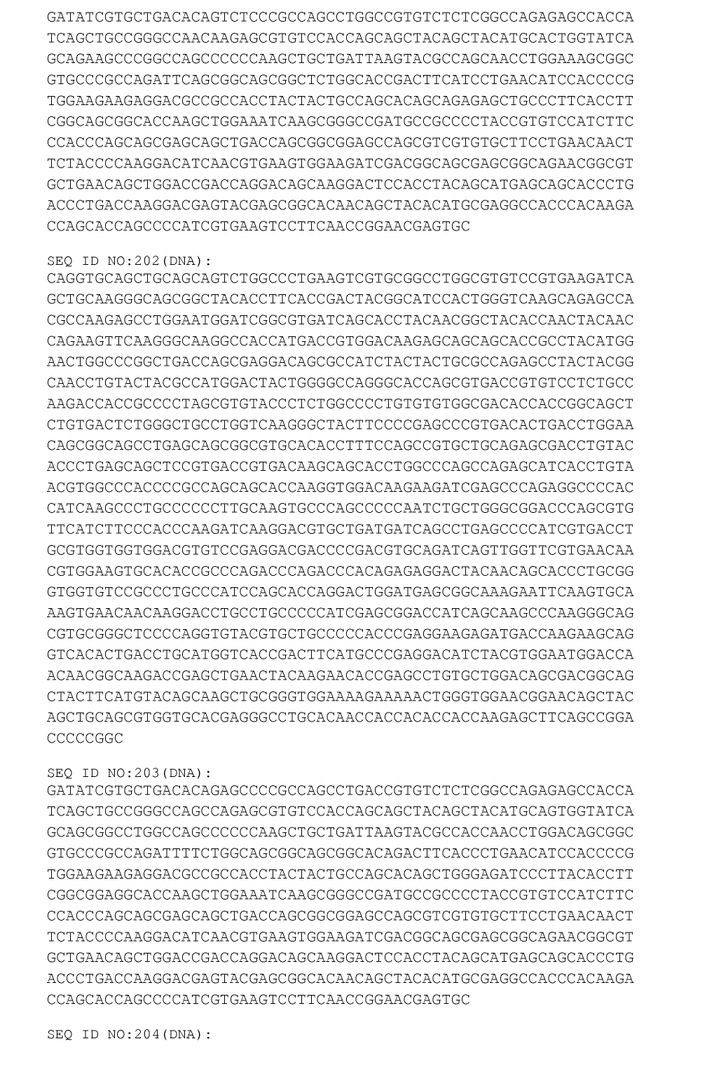

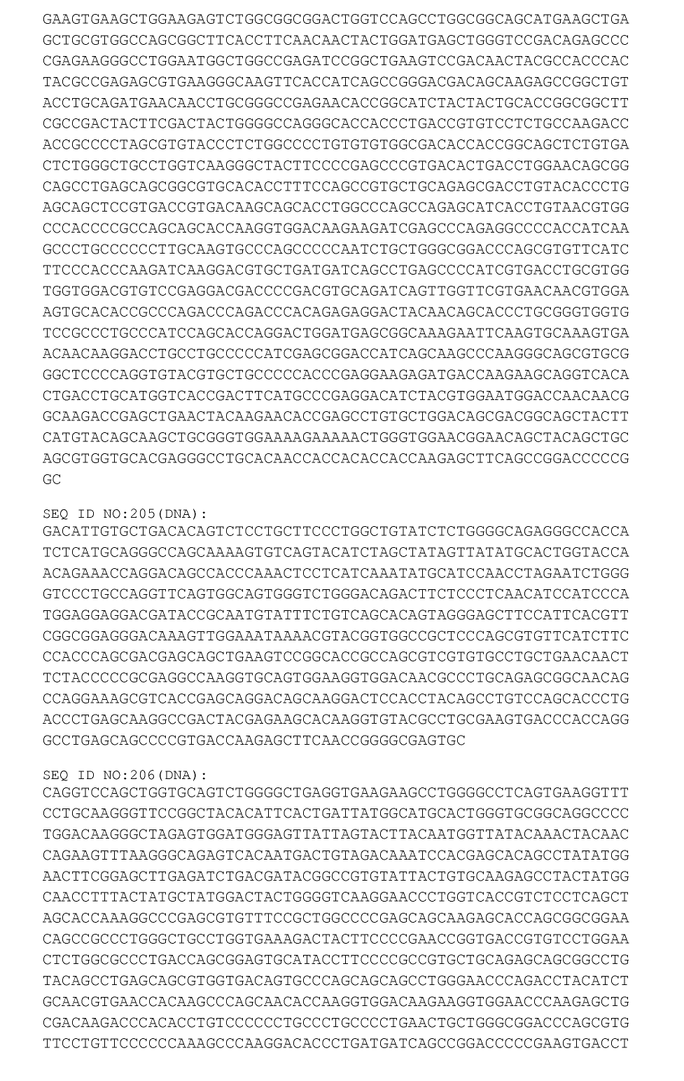

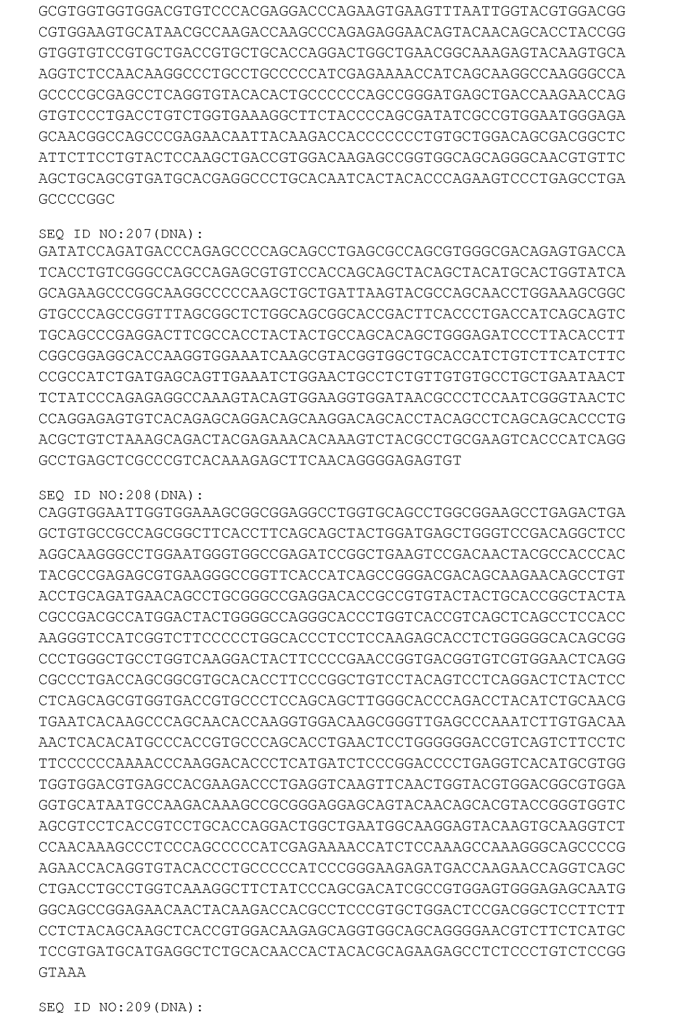

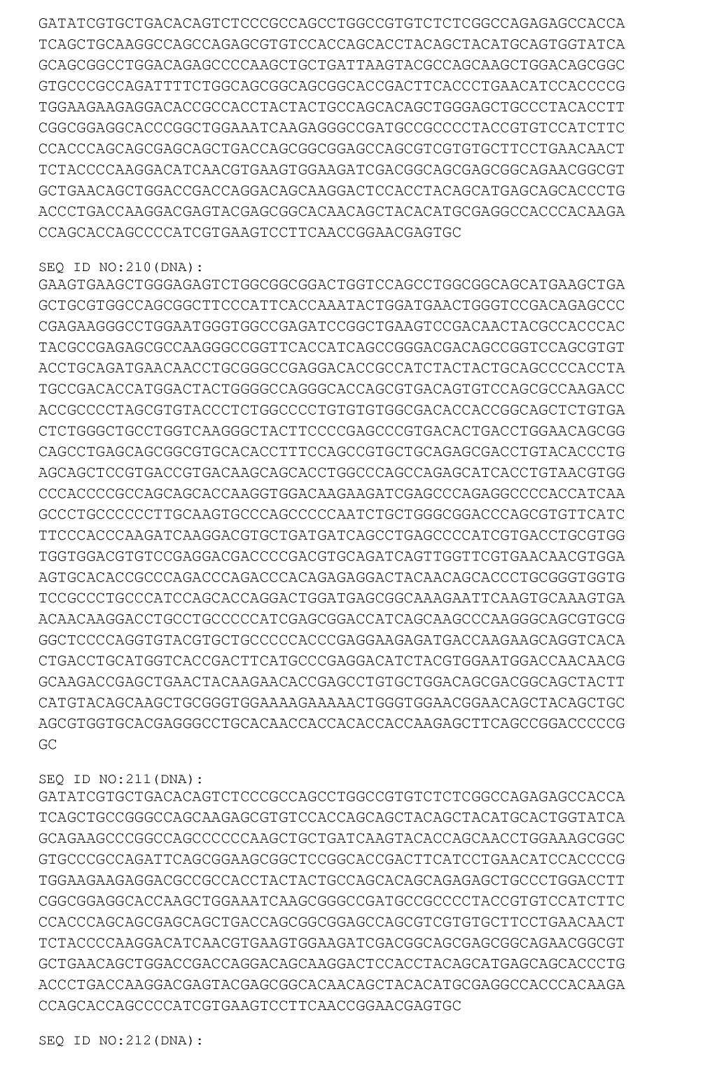

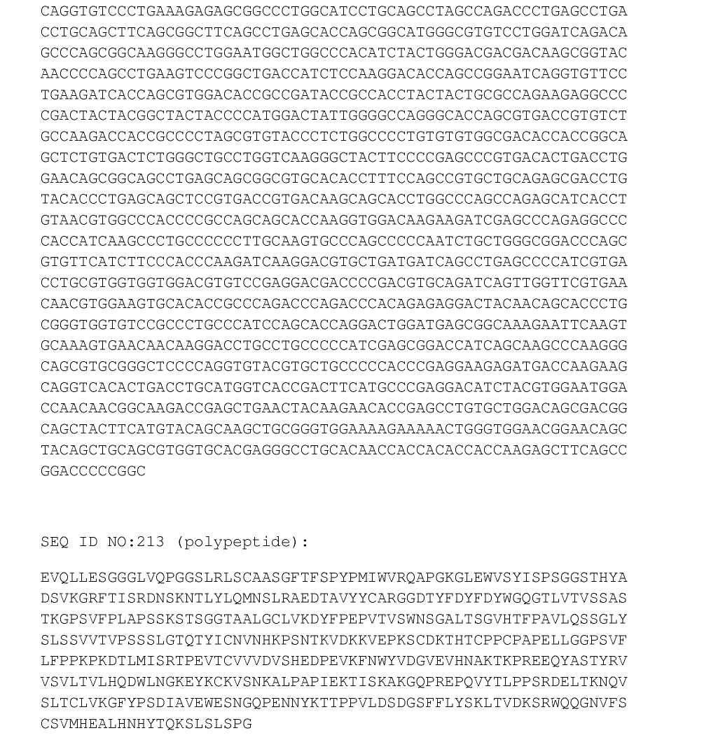

Amino acid at position 47 (D47) of TWEAKR (SEQ ID NO:169) is regarded as critical for binding for the antibodies of the invention, which means the antibody specifically binds to the D at position 47 (D47) of TWEAKR (SEQ ID NO:169), when the antibody loses more than 20%, alternatively more than 30%, alternatively more than 40%, alternatively more than 50%, alternatively more than 60%, alternatively more than 70%, alternatively more than 80%, alternatively more than 90%, alternatively 100%, of its ELISA signal by changing this residue into an Alanine as described in Example 2 and FIG. 6. Alternatively, an antibody specifically binds to the D at position 47 (D47) of TWEAKR (SEQ ID NO:169), when the antibody loses more than 20%, alternatively more than 30%, alternatively more than 40%, alternatively more than 50%, alternatively more than 60%, alternatively more than 70%, alternatively more than 80%, alternatively more than 90%, alternatively 100%, of its ELISA signal on TPP-2614 compared to TPP-2203. Preferably, an antibody specifically binds to the D at position 47 (D47) of TWEAKR (SEQ ID NO:169), when the antibody loses more than 80% of its ELISA signal on TPP-2614 compared to TPP-2203.

A preferred embodiment of the invention is an anti-TWEAKR antibody or antigen-binding fragment thereof, which specifically binds to aspartate 47 (D47) of TWEAKR (SEQ ID NO:169).

A further preferred embodiment of the invention is an agonistic anti-TWEAKR antibody or antigen-binding fragment thereof, which specifically binds to aspartate 47 (D47) of TWEAKR (SEQ ID NO:169).

A further preferred embodiment of the invention is an agonistic anti-TWEAKR antibody or antigen-binding fragment thereof, which has reduced ADCC activity or which lacks ADCC activity, and which specifically binds to aspartate 47 (D47) of TWEAKR (SEQ ID NO:169). A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is selected from the group of agonistic activities consisting of induction of Caspase3/7, inhibition of proliferation of TWEAKR expressing cell lines, and induction of cytokine secretion.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of Caspase3/7.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of Caspase3/7 in a TWEAKR expressing cancer cell line.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of Caspase3/7 in a TWEAKR expressing cancer cell line comprised in the group consisting of WiDr, A253, NCI-H322, HT-29 and 786-O cells.

A further more preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is higher induction of Caspase3/7 in a HT-29 and/or 786-O cell line compared to the induction by recombinant human TWEAK. In a further preferred embodiment the concentration of anti-TWEAKR antibody used is 100 μg/ml and of recombinant human TWEAK is 300 ng/ml.

It is another embodiment of the invention to provide antibodies, or antigen-binding antibody fragments thereof, or variants thereof, which bind specifically to the cysteine rich domain (aa 34-68 of SEQ ID:169) of TWEAKR of different species (FIG. 1).

It is another preferred embodiment of the invention to provide antibodies, or antigen-binding antibody fragments thereof, or variants thereof, which bind specifically to the cysteine rich domain (aa 34-68 of SEQ ID:169) of TWEAKR and which bind specifically to the D at position 47 (D47) of TWEAKR.

It is another preferred embodiment of the invention to provide antibodies, or antigen-binding antibody fragments thereof, or variants thereof, which bind specifically to the cysteine rich domain (aa 34-68 of SEQ ID:169) of TWEAKR of at least two species comprised in the group TWEAKR species consisting of human, mouse, dog, pig, rat, and macaca fascicularis and which bind specifically to the D at position 47 (D47) of TWEAKR. In a preferred embodiment the two species are human and mouse.

It is another embodiment of the invention to provide antibodies, or antigen-binding antibody fragments thereof, or variants thereof, which inhibit the proliferation of different TWEAKR expressing cell lines. In line with the strong induction of Caspase 3/7 an efficacious inhibition of proliferation of different cancer cell lines is observed. The antibodies of the current invention are more efficacious as compared to other known antibodies (PDL-192, P4A8) in inhibiting proliferation of various cancer cells. In most experiments the antibodies of the current invention show a higher efficacy or the same efficacy as compared to TWEAK ligand. Thus, the antibodies are unique in their efficacy to induce apoptosis and proliferation inhibition in a broad panel of cancer cell lines including but not limited to 786-O, LOVO, NCI-H1975, SW480, WiDr, HT-29, A253, SK-OV3.

A further preferred embodiment of the invention is an agonistic anti-TWEAKR antibody or antigen-binding fragment thereof, which specifically binds to aspartate 47 (D47) of TWEAKR (SEQ ID N0:169) wherein the agonistic activity of the anti-TWEAKR antibody is inhibition of proliferation of TWEAKR expressing cell lines. In a preferred embodiment of the invention the TWEAKR expressing cell line is comprised in the group consisting of 786-O, LOVO, NCI-H1975, SW480, WiDr, HT-29, A253, and SK-OV3.

A further more preferred embodiment of the invention is an agonistic anti-TWEAKR antibody or antigen-binding fragment thereof, which specifically binds to aspartate 47 (D47) of TWEAKR (SEQ ID N0:169) wherein the agonistic activity of the anti-TWEAKR antibody is stronger inhibition of proliferation of a786-O and/or WiDr cell line compared to the inhibition by recombinant human TWEAK. In a further preferred embodiment the concentration of anti-TWEAKR antibody used is 100 μg/ml and of recombinant human TWEAK is 300 ng/ml.

It is another embodiment of the invention to provide antibodies, or antigen-binding antibody fragments thereof, or variants thereof, which strongly induce cytokine secretion from various cancer cells including but not limited to A375, WiDr cells and xenografts. Cytokines induced include but are not limited to IL-8, IL-15, IP-10, IL-1RA and MCP-1. A preferred cytokine which is induced is IL-8. Antibodies of this invention show a higher efficacy to induce IL-8 in A375 cells compared to other known antibodies (PDL-192(TPP-1104), P4A8(TPP-1324), 136.1(TPP-2194))

A preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion.

A preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion in a TWEAKR expressing cancer cell line. In a more preferred embodiment the TWEAKR expressing cancer cell line is a A375 or a WiDr cell line

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion wherein the cytokine is comprised in a group of cytokine consisting of IL-8, IL-15, IP-10, IL-1RA and MCP-1.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion in a TWEAKR expressing cancer cell line secretion wherein the cytokine is comprised in a group of cytokine consisting of IL-8, IL-15, IP-10, IL-1RA and MCP-1.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion in a TWEAKR expressing cancer cell line secretion wherein the cytokine is comprised in a group of cytokine consisting of IL-8, IL-15, IP-10, IL-1RA and MCP-1 and wherein the TWEAKR expressing cancer cell line is a A375 or a WiDr cell line.

In a further preferred embodiment the cytokine is IL-8, in an even more preferred embodiment the IL-8 is human IL-8.

A preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of cytokine secretion in a mouse tumor xenograft model. In a further preferred embodiment the secreted cytokine is a human cytokine derived from the tumor xenograft.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of human IL-8 secretion in a mouse tumor xenograft model.

In a further preferred embodiment the mouse tumor xenograft model is a A375 or WiDr mouse xenograft model.

In a further preferred embodiment the induction of cytokine secretion is observed after injection of at 3 mg/kg or higher or 10 mg/kg or higher anti-TWEAKR antibody of the invention.

A further preferred embodiment of the invention is an agonistic anti-TWEAKR antibody or antigen-binding fragment thereof, which specifically binds to aspartate 47 (D47) of TWEAKR (SEQ ID NO:169) wherein the agonistic activity of the anti-TWEAKR antibody is induction of human IL-8 secretion in a mouse WiDr tumor xenograft model after injection of 3 mg/kg of said antibody wherein no induction of the mouse IL-8 analogue KC is detected.

In a further preferred embodiment the induction of cytokine secretion is observed in the plasma of tumor bearing mice.

It is another embodiment of the invention to provide antibodies, or antigen-binding antibody fragments thereof, or variants thereof, which bind to a broad range of different TWEAKR expressing cell lines including, but not limited to the ones shown in Table 21. The examples in Table 21 include human and murine cell lines from many tumor origins (e.g. NSCLC, CRC, HNSCC, RCC, PancCA, OvCa, BreastCA, Melanoma, GastricCA, Esophageal CA, Bladder CA, HCC, Prostate CA, Neuroblastoma).

It is another embodiment of the invention to provide antibodies, or antigen-binding antibody fragments thereof, or variants thereof that are safe for human administration.

It is another embodiment of the invention to provide antibodies, or antigen-binding antibody fragments thereof, or variants thereof, which bind to human TWEAKR and are cross-reactive to TWEAKR of another species including, but not limited to murine, rat, pig, dog, macaca fascicularis with similar affinity. Preferably, said other species is a rodent, such as for example mouse or rat. Most preferably, the antibodies, or antigen-binding antibody fragments thereof, or variants thereof bind to human TWEAKR and are cross-reactive to murine TWEAKR.

It is another embodiment of the invention to provide antibodies which constitute a tool for diagnosis of malignant or dysplastic conditions in which TWEAKR expression is elevated compared to normal tissue or where TWEAKR is shed from the cell surface and becoming detectable in serum. Provided are anti-TWEAKR antibodies conjugated to a detectable marker. Preferred markers are a radiolabel, an enzyme, a chromophore or a fluorescer.

Throughout this document, reference is made to the following preferred antibodies of the invention as depicted in Table 31: “TPP-2090”, “TPP-2149”, “TPP-2093”, “TPP-2148”, “TPP-2084”, “TPP-2077”, “TPP-1538”, “TPP-883”, “TPP-1854”, “TPP-1853”, “TPP-1857”, “TPP-1858”, and “TPP-2658”.

TPP-2090 represents an antibody comprising a heavy chain region corresponding to SEQ ID NO: 2 and a light chain region corresponding to SEQ ID NO: 1.

TPP-2149 represents an antibody comprising a heavy chain region corresponding to SEQ ID NO: 12 and a light chain region corresponding to SEQ ID NO: 11.

TPP-2093 represents an antibody comprising a heavy chain region corresponding to SEQ ID NO: 22 and a light chain region corresponding to SEQ ID NO: 21.

TPP-2148 represents an antibody comprising a heavy chain region corresponding to SEQ ID NO: 32 and a light chain region corresponding to SEQ ID NO: 31.

TPP-2084 represents an antibody comprising a heavy chain region corresponding to SEQ ID NO: 42 and a light chain region corresponding to SEQ ID NO: 41.

TPP-2077 represents an antibody comprising a heavy chain region corresponding to SEQ ID NO: 52 and a light chain region corresponding to SEQ ID NO: 51.

TPP-1538 represents an antibody comprising a heavy chain region corresponding to SEQ ID NO: 62 and a light chain region corresponding to SEQ ID NO: 61.

TPP-883 represents an antibody comprising a heavy chain region corresponding to SEQ ID NO: 72 and a light chain region corresponding to SEQ ID NO: 71.

TPP-1854 represents an antibody comprising a heavy chain region corresponding to SEQ ID NO: 82 and a light chain region corresponding to SEQ ID NO: 81.

TPP-1853 represents an antibody comprising a heavy chain region corresponding to SEQ ID NO: 92 and a light chain region corresponding to SEQ ID NO: 91.

TPP-1857 represents an antibody comprising a heavy chain region corresponding to SEQ ID NO: 102 and a light chain region corresponding to SEQ ID NO: 101.

TPP-1858 represents an antibody comprising a heavy chain region corresponding to SEQ ID NO: 112 and a light chain region corresponding to SEQ ID NO: 111.

TPP-2658 represents an antibody comprising a heavy chain region corresponding to SEQ ID NO: 213 and a light chain region corresponding to SEQ ID NO: 1.

TPP-2090 represents an antibody comprising a variable heavy chain region corresponding to SEQ ID NO: 10 and a variable light chain region corresponding to SEQ ID NO: 9.

TPP-2149 represents an antibody comprising a variable heavy chain region corresponding to SEQ ID NO: 20 and a variable light chain region corresponding to SEQ ID NO: 19.

TPP-2093 represents an antibody comprising a variable heavy chain region corresponding to SEQ ID NO: 30 and a variable light chain region corresponding to SEQ ID NO: 29.

TPP-2148 represents an antibody comprising a variable heavy chain region corresponding to SEQ ID NO: 40 and a variable light chain region corresponding to SEQ ID NO: 39.

TPP-2084 represents an antibody comprising a variable heavy chain region corresponding to SEQ ID NO: 50 and a variable light chain region corresponding to SEQ ID NO: 49.

TPP-2077 represents an antibody comprising a variable heavy chain region corresponding to SEQ ID NO: 60 and a variable light chain region corresponding to SEQ ID NO: 59.

TPP-1538 represents an antibody comprising a variable heavy chain region corresponding to SEQ ID NO: 70 and a variable light chain region corresponding to SEQ ID NO: 69.

TPP-883 represents an antibody comprising a variable heavy chain region corresponding to SEQ ID NO: 80 and a variable light chain region corresponding to SEQ ID NO: 79.

TPP-1854 represents an antibody comprising a variable heavy chain region corresponding to SEQ ID NO: 90 and a variable light chain region corresponding to SEQ ID NO: 89.

TPP-1853 represents an antibody comprising a variable heavy chain region corresponding to SEQ ID NO: 100 and a variable light chain region corresponding to SEQ ID NO: 99.

TPP-1857 represents an antibody comprising a variable heavy chain region corresponding to SEQ ID NO: 110 and a variable light chain region corresponding to SEQ ID NO: 109.

TPP-1858 represents an antibody comprising a variable heavy chain region corresponding to SEQ ID NO: 120 and a variable light chain region corresponding to SEQ ID NO: 119.

In a further preferred embodiment the antibodies or antigen-binding fragments comprise heavy or light chain CDR sequences which are at least 50%, 55%, 60% 70%, 80%, 90, or 95% identical to at least one, preferably corresponding, CDR sequence of the antibodies “TPP-2090”, “TPP-2149”, “TPP-2093”, “TPP-2148”, “TPP-2084”, “TPP-2077”, “TPP-1538”, “TPP-883”, “TPP-1854”, “TPP-1853”, “TPP-1857” or “TPP-1858” or at least 50%, 60%, 70%, 80%, 90%, 92% or 95% identical to the VH or VL sequence of “TPP-2090”, “TPP-2149”, “TPP-2093”, “TPP-2148”, “TPP-2084”, “TPP-2077”, “TPP-1538”, “TPP-883”, “TPP-1854”, “TPP-1853”, “TPP-1857” or “TPP-1858”, respectively.

In a further preferred embodiment the antibody or antigen-binding fragment of the invention comprises at least one CDR sequence or at least one variable heavy chain or variable light chain sequence as depicted in Table 31.

In a more preferred embodiment the antibody of the invention or antigen-binding fragment thereof comprises a heavy chain antigen-binding region that comprises SEQ ID NO:6 (H-CDR1), SEQ ID NO:7 (H-CDR2) and SEQ ID NO:8 (H-CDR3) and comprises a light chain antigen-binding region that comprises SEQ ID NO:3 (L-CDR1), SEQ ID NO:4 (L-CDR2) and SEQ ID NO:5 (L-CDR3).

In a more preferred embodiment the antibody of the invention or antigen-binding fragment thereof comprises a heavy chain antigen-binding region that comprises SEQ ID NO:16 (H-CDR1), SEQ ID NO:17 (H-CDR2) and SEQ ID NO:18 (H-CDR3) and comprises a light chain antigen-binding region that comprises SEQ ID NO:13 (L-CDR1), SEQ ID NO:14 (L-CDR2) and SEQ ID NO:15 (L-CDR3).

In a more preferred embodiment the antibody of the invention or antigen-binding fragment thereof comprises a heavy chain antigen-binding region that comprises SEQ ID NO:26 (H-CDR1), SEQ ID NO:27 (H-CDR2) and SEQ ID NO:28 (H-CDR3) and comprises a light chain antigen-binding region that comprises SEQ ID NO:23 (L-CDR1), SEQ ID NO:24 (L-CDR2) and SEQ ID NO:25 (L-CDR3).

In a more preferred embodiment the antibody of the invention or antigen-binding fragment thereof comprises a heavy chain antigen-binding region that comprises SEQ ID NO:36 (H-CDR1), SEQ ID NO:37 (H-CDR2) and SEQ ID NO:38 (H-CDR3) and comprises a light chain antigen-binding region that comprises SEQ ID NO:33 (L-CDR1), SEQ ID NO:34 (L-CDR2) and SEQ ID NO:35 (L-CDR3).

In a more preferred embodiment the antibody of the invention or antigen-binding fragment thereof comprises a heavy chain antigen-binding region that comprises SEQ ID NO:46 (H-CDR1), SEQ ID NO:47 (H-CDR2) and SEQ ID NO:48 (H-CDR3) and comprises a light chain antigen-binding region that comprises SEQ ID NO:43 (L-CDR1), SEQ ID NO:44 (L-CDR2) and SEQ ID NO:45 (L-CDR3).

In a more preferred embodiment the antibody of the invention or antigen-binding fragment thereof comprises a heavy chain antigen-binding region that comprises SEQ ID NO:56 (H-CDR1), SEQ ID NO:57 (H-CDR2) and SEQ ID NO:58 (H-CDR3) and comprises a light chain antigen-binding region that comprises SEQ ID NO:53 (L-CDR1), SEQ ID NO:54 (L-CDR2) and SEQ ID NO:55 (L-CDR3).

In a more preferred embodiment the antibody of the invention or antigen-binding fragment thereof comprises a heavy chain antigen-binding region that comprises SEQ ID NO:66 (H-CDR1), SEQ ID NO:67 (H-CDR2) and SEQ ID NO:68 (H-CDR3) and comprises a light chain antigen-binding region that comprises SEQ ID NO:63 (L-CDR1), SEQ ID NO:64 (L-CDR2) and SEQ ID NO:65 (L-CDR3).

In a more preferred embodiment the antibody of the invention or antigen-binding fragment thereof comprises a heavy chain antigen-binding region that comprises SEQ ID NO:76 (H-CDR1), SEQ ID NO:77 (H-CDR2) and SEQ ID NO:78 (H-CDR3) and comprises a light chain antigen-binding region that comprises SEQ ID NO:73 (L-CDR1), SEQ ID NO:74 (L-CDR2) and SEQ ID NO:75 (L-CDR3).

In a more preferred embodiment the antibody of the invention or antigen-binding fragment thereof comprises a heavy chain antigen-binding region that comprises SEQ ID NO:86 (H-CDR1), SEQ ID NO:87 (H-CDR2) and SEQ ID NO:88 (H-CDR3) and comprises a light chain antigen-binding region that comprises SEQ ID NO:83 (L-CDR1), SEQ ID NO:84 (L-CDR2) and SEQ ID NO:85 (L-CDR3).

In a more preferred embodiment the antibody of the invention or antigen-binding fragment thereof comprises a heavy chain antigen-binding region that comprises SEQ ID NO:96 (H-CDR1), SEQ ID NO:97 (H-CDR2) and SEQ ID NO:98 (H-CDR3) and comprises a light chain antigen-binding region that comprises SEQ ID NO:93 (L-CDR1), SEQ ID NO:94 (L-CDR2) and SEQ ID NO:95 (L-CDR3).

In a more preferred embodiment the antibody of the invention or antigen-binding fragment thereof comprises a heavy chain antigen-binding region that comprises SEQ ID NO:106 (H-CDR1), SEQ ID NO:107 (H-CDR2) and SEQ ID NO:108 (H-CDR3) and comprises a light chain antigen-binding region that comprises SEQ ID NO:103 (L-CDR1), SEQ ID NO:104 (L-CDR2) and SEQ ID NO:105 (L-CDR3).

In a more preferred embodiment the antibody of the invention or antigen-binding fragment thereof comprises a heavy chain antigen-binding region that comprises SEQ ID NO:116 (H-CDR1), SEQ ID NO:117 (H-CDR2) and SEQ ID NO:118 (H-CDR3) and comprises a light chain antigen-binding region that comprises SEQ ID NO:113 (L-CDR1), SEQ ID NO:114 (L-CDR2) and SEQ ID NO:115 (L-CDR3).

Sequence alignment of the CDRs of the antibodies of this invention reveals a consensus sequence (see FIG. 24). In a more preferred embodiment the antibodies of the invention or antigen-binding fragment thereof comprise:

a variable heavy chain comprising
a heavy chain CDR1 encoded by an amino acid sequence comprising the formula PYPMX (SEQ ID NO: 171), wherein X is I or M;
a heavy chain CDR2 encoded by an amino acid sequence comprising the formula YISPSGGXTHYADSVKG (SEQ ID NO: 172), wherein X is S or K; and
a heavy chain CDR3 encoded by an amino acid sequence comprising the formula GGDTYFDYFDY (SEQ ID NO: 173);

and a variable light chain comprising
a light chain CDR1 encoded by an amino acid sequence comprising the formula RASQSISXYLN (SEQ ID NO: 174), wherein X is G or S;
a light chain CDR2 encoded by an amino acid sequence comprising the formula XASSLQS (SEQ ID NO: 175), wherein X is Q, A, or N; and
a light chain CDR3 encoded by an amino acid sequence comprising the formula QQSYXXPXIT (SEQ ID NO: 176), wherein X at position 5 is T or S, and X at position 6 is T or S, and X at position 8 is G, or F.

Antibodies differ in sequence, not only within their complementarity determining regions (CDRs), but also in the framework (FR). These sequence differences are encoded in the different V-genes. The human antibody germline repertoire has been completely sequenced. There are about 50 functional VH germline genes which can be grouped into six subfamilies according to sequence homology VH1, VH2, VH3, VH4, VH5 and VH6 (Tomlinson et al., 1992, J. Mol. Biol. 227, 776-798; Matsuda & Honjo, 1996, Advan. Immunol. 62, 1-29). About 40 functional VL kappa genes comprising seven subfamilies are known (Cox et al., 1994, Eur. J. Immunol. 24, 827-836; Barbie & Lefranc, 1998, Exp. Clin. Immunogenet. 15, 171-183). Vkappa1, Vkappa2, Vkappa3, Vkappa4, Vkappa5, Vkappa6 and Vkappa7. Disclosed herein are heavy chains of antibodies of this invention that belong to the human VH3 subfamily and the light chains of antibodies of this invention that belong to the human Vkappa1 subfamily, respectively. It is known that framework sequences of antibodies belonging to the same subfamily are closely related, e.g. antibodies comprising a human Vh3 subfamily member all share comparable stability (Honegger et al, 2009, Protein Eng Des Sel. 22(3):121-134). It is well known in the art that CDRs from antibodies can be grafted on different frameworks while maintaining special features of the corresponding origin antibody. CDRs have been successfully grafted on frameworks belonging to a different species as well as on frameworks of the same species belonging to a different subfamily. In a further embodiment the antibody or antigen-binding fragment of the invention comprises at least one CDR sequence of an antibody of the invention as depicted in Table 31 and a human variable chain framework sequence.

In a preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 31 and a human variable light and human variable heavy chain framework sequence.

In a more preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 31 and a human VH3 subfamily framework sequence for the variable heavy chain and a human Vkappa 1 subfamily framework sequence for the variable light chain. In a more preferred embodiment the human VH3 subfamily framework sequence for the variable heavy chain is comprised in the group of VH3 subfamily framework sequence consisting of VH3-07, VH3-09, VH3-11, VH3-13, VH3-15, VH3-20, VH3-21, VH3-23, VH3-30, VH3-30.3, VH3-30.5, VH3-33, VH3-43, VH3-48, VH3-49, VH3-53, VH3-64, VH3-66, VH3-72, VH3-73, VH3-74 and VH3-d. In an even more preferred embodiment the human VH3 framework sequence has less than 16 or less than 15 amino acid exchanges compared to a human VH3-23 framework sequence. In a more preferred embodiment the human Vkappa1 subfamily framework sequence for the variable light chain is comprised in the group of Vkappa1 subfamily framework sequence consisting of Vkappa 1-5, Vkappa 1-6, Vkappa 1-8, Vkappa 1D-8, Vkappa 1-9, Vkappa 1-12, Vkappa 1D-12, Vkappa 1-13, Vkappa 1D-13, Vkappa 1-16, Vkappa 1D-16, Vkappa 1-17, Vkappa 1D-17, Vkappa 1-27, Vkappa 1-33, Vkappa 1D-33, Vkappa 1-37, Vkappa 1D-37, Vkappa 1-39, Vkappa 1D-39, Vkappa 1D-42, Vkappa 1D-43. In an even more preferred embodiment the human Vkappa 1 framework sequence has less than 15 or less than 13 amino acid exchanges compared to a human Vkappa 1-39 framework sequence.

In a more preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 31 and a human VH3 subfamily framework sequence for the variable heavy chain and a human Vkappa 1-39 framework sequence for the variable light chain.

In a most preferred embodiment the antibody or antigen-binding fragment of the invention comprises a variable light chain or light chain antigen-binding region comprising the L-CDR1, L-CDR2 and L-CDR3 sequence of the variable light chain and a variable heavy chain or heavy chain antigen-binding region comprising the H-CDR1, H-CDR2 and H-CDR3 sequence of the variable heavy chain antibody of the invention as depicted in Table 31 and a human VH3-3 framework sequence for the variable heavy chain and a human Vkappa 1-39 framework sequence for the variable light chain.

In a preferred embodiment the variable light chain framework sequence belongs to the human Vkappa1 subfamily and the variable heavy chain framework sequence belongs to the human VH3 subfamily. A VH3 subfamily or Vkappa1 subfamily variable chain framework sequence may comprises sequence variations compared to the respective WT framework sequence to adopt the framework for insertion of the respective CDR sequence. In a further embodiment a VH3 subfamily or Vkappa1 subfamily variable chain framework sequence comprising a sequence variation compared to the WT framework sequence is a VH3 subfamily member or Vkappa1 subfamily member, respectively. Preferably, such a variant framework sequence has up to 15 sequence variations, more preferably up to 10 sequence variations, more preferably up to 5 sequence variations, most preferably up to 3 sequence variations.

An antibody of the invention may be an IgG (e.g. IgG1 IgG2, IgG3, IgG4), while an antibody fragment may be a Fab, Fab′, F(ab′)2 or scFv, for example. An inventive antibody fragment, accordingly, may be, or may contain, an antigen-binding region that behaves in one or more ways as described herein.

In a preferred embodiment the antibodies or antigen-binding antibody fragments of the invention are monoclonal. In a further preferred embodiment the antibodies or antigen-binding antibody fragments of the invention are human, humanized or chimeric.

In another aspect, the invention provides antibodies or antigen-binding fragments thereof having an antigen-binding region that binds specifically to and/or has a high affinity for TWEAKR. An antibody or antigen-binding fragment is said to have a “high affinity” for an antigen if the affinity measurement is less than 250 nM (monovalent affinity of the antibody or antigen-binding fragment). An inventive antibody or antigen-binding region preferably can bind to human TWEAKR with an affinity of less than 250 nM, preferably less than 150 nM, more preferably less than 100 nM, more preferably less than 50 nM, more preferably less than 30 nM, more preferably less than 20 nM, determined as monovalent affinity to human TWEAKR (see Example 2) as shown in Table 6.

In another aspect, the invention provides antibodies or antigen-binding fragments thereof having an antigen-binding region that binds specifically to TWEAKR and does not bind to other members of the TNF receptor superfamily (see Table 20) as shown in FIG. 11 exemplarily for TPP-2090.

The IgG1 format was used for the cell-based affinity assessment by fluorescence-activated cell sorting (FACS). Table 21 provides exemplarily for TPP-2090 and TPP-1538 a summary of the binding of representative anti-TWEAKR antibodies on cancer cell lines of human and murine origin. The maximal cellular binding of the antibodies as detected by FACS analysis of the invention is moderate as compared to other described antibodies but nevertheless these antibodies have a very strong agonistic activity underlining the importance of the novel epitope found for the antibodies of the invention.

It is another embodiment of the invention to provide antibodies, or antigen-binding antibody fragments thereof, or variants thereof, which are internalized efficiently following binding to a TWEAKR expressing cell. An antibody of the invention might be co-administered with known medicaments, and in some instances the antibody might itself be modified. For example, an antibody could be conjugated to a cytotoxic agent, immunotoxin, toxophore or radioisotope to potentially further increase efficacy.

An antibody or antigen-binding fragment of the invention internalizes “efficiently” when its time of half maximal internalization (t 1/2) as measured by granule count/cell into TWEAKR expressing tumor cells is shorter than 400 min or more preferably shorter than 300 min and still more preferably shorter than 200 min. Further preferred are antibodies or antigen-binding fragments with half maximal internalization times (t 1/2) of 100 minutes or less as determined by the protocol described in Example 7 and FIG. 17.

Internalizable antibodies of the invention or antigen-binding fragments thereof are suitable as targeting moiety of an antibody-drug conjugate (ADC). An antibody or antigen-binding fragment is suitable in an in vitro or in vivo method to deliver a compound, preferably a cytotoxic agent, into a TWEAKR expressing cell. The efficient internalization is shown with fluorescently labeled antibodies (Example 7). The efficient use as an antibody drug conjugate is exemplified with a Saporin-conjugated antibody (Example 7).

In some embodiments antibodies of the invention or antigen-binding fragments thereof, or nucleic acids encoding the same are isolated. An isolated biological component (such as a nucleic acid molecule or protein such as an antibody) is one that has been substantially separated or purified away from other biological components in the cell of the organism in which the component naturally occurs, e.g., other chromosomal and extra-chromosomal DNA and RNA, proteins and organdies. Nucleic acids and proteins that have been “isolated” include nucleic acids and proteins purified by standard purification methods as described for example in Sambrook et al., 1989 (Sambrook, J., Fritsch, E. F. and Maniatis, T. (1989) Molecular Cloning: A laboratory manual, Cold Spring Harbor Laboratory Press, Cold Spring Harbor, USA) and Robert K. Scopes et al. 1994 (Protein Purification,—Principles and Practice, Springer Science and Business Media LLC). The term also embraces nucleic acids and proteins prepared by recombinant expression in a host cell as well as chemically synthesized nucleic acids.

An antibody of the invention may be derived from a recombinant antibody library that is based on amino acid sequences that have been isolated from the antibodies of a large number of healthy volunteers e.g. using the n-CoDeR® technology the fully human CDRs are recombined into new antibody molecules. Or alternatively antibody libraries as the fully human antibody phage display library described in Hoet R M et al, Nat Biotechnol 2005; 23(3):344-8) can be used to isolate TWEAKR-specific antibodies.

### Antibody Generation

A fully human antibody phage display library (Hoet R M et al, Nat Biotechnol 2005; 23(3):344-8) was used to isolate TWEAKR-specific, human monoclonal antibodies of the present invention by protein panning (Hoogenboom H. R., Nat Biotechnol 2005; 23(3):1105-16) with dimeric Fc-fused extracellular domains of human and murine TWEAKR as immobilized target.

11 different Fab-phages were identified and the corresponding antibodies were re-cloned into a mammalian IgG expression vector which provides the missing CH2-CH3 domains not present in the soluble Fab. After identification of preferred antibodies these were expressed as full length IgGs. Theses constructs were for example transiently expressed in mammalian cells as described in Tom et al., Chapter 12 in Methods Express: Expression Systems edited by Micheal R. Dyson and Yves Durocher, Scion Publishing Ltd, 2007 (see Example 1). The antibodies were purified by Protein A chromatography and further characterized by their binding affinity to soluble monomeric TWEAKR in ELISA and BIAcore analysis as described in Example 2. To determine the cell binding characteristics of anti-TWEAKR antibodies, binding was tested by flow cytometry to a panel of cell lines (HT29, HS68, HS578).

NF-kappaB reporter gene assays were performed to assess the agonistic activity of all 11 identified antibodies (human IgG1). The antibody with the strongest in vitro efficacy (TPP-883) was selected for further potency and affinity maturation (see Example 1 for details). 1 single substitution variant was detected with improved agonistic activity: G102T of CDR-H3. Finally, 7 variants were selected based on enhanced affinity compared to the best single substitution variant, G102T. The corresponding DNA of these were re-cloned in a mammalian IgG expression vector and tested for functional activity in the afore mentioned NFkB reporter cell assay. Finally, the obtained sequences were compared with human germline sequences and deviations without significant impact on affinity and potency were adjusted. The following antibodies were obtained by antibody library screening and by affinity and/or potency maturation: “TPP-2090”, “TPP-2149”, “TPP-2093”, “TPP-2148”, “TPP-2084”, “TPP-2077”, “TPP-1538”, “TPP-883”, “TPP-1854”, “TPP-1853”, “TPP-1857”, and “TPP-1858”.

Antibodies of the invention can be further generated by methods known in the art like antibody phage display screening (for example see Hoet R M et al, Nat Biotechnol 2005; 23(3):344-8), the well-established hybridoma technology (for example see Köhler and Milstein Nature. 1975 Aug. 7; 256(5517):495-7), or immunization of mice inter alia immunization of hMAb mice (e.g. Veloclmmune Mouse®).

### Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies disclosed herein, while appreciating these variants having the ability to bind to TWEAKR fall within the scope of the present invention.

A variant can include, for example, an antibody that has at least one altered complementary determining region (CDR) (hyper-variable) and/or framework (FR) (variable) domain/position, vis-à-vis a peptide sequence disclosed herein. To better illustrate this concept, a brief description of antibody structure follows.

An antibody is composed of two peptide chains, each containing one (light chain) or three (heavy chain) constant domains and a variable region (VL, VH), the latter of which is in each case made up of four FR regions and three interspaced CDRs. The antigen-binding site is formed by one or more CDRs, yet the FR regions provide the structural framework for the CDRs and, hence, play an important role in antigen binding. By altering one or more amino acid residues in a CDR or FR region, the skilled worker routinely can generate mutated or diversified antibody sequences, which can be screened against the antigen, for new or improved properties, for example.

A further preferred embodiment of the invention is an antibody or antigen-binding fragment in which the VH and VL sequences are selected as shown in Table 31. The skilled worker can use the data in Table 31 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Alternatively, the skilled worker could make the same analysis by comparing the amino acid sequences disclosed herein to known sequences of the same class of such antibodies, using, for example, the procedure described by Knappik A., et al., JMB 2000, 296:57-86.

Furthermore, variants may be obtained by using one antibody as starting point for further optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done e.g. by synthesizing a collection of DNA molecules using trinucleotide mutagenesis (TRIM) technology (Virnekäs B. et al., Nucl. Acids Res. 1994, 22: 5600). Antibodies or antigen-binding fragments thereof include molecules with modifications/variations including but not limited to e.g. modifications leading to altered half-life (e.g. modification of the Fc part or attachment of further molecules such as PEG), altered binding affinity or altered ADCC or CDC activity.

One embodiment of an antibody is TPP-2658, which includes a modification resulting in altered ADCC. TPP-2658 has a mutation in the Fc part at N297 (compared to TPP-2090) resulting in an aglycosylated antibody variant lacking ADCC.

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

As used herein, “sequence identity” between two polypeptide sequences, indicates the percentage of amino acids that are identical between the sequences. “Sequence homology” indicates the percentage of amino acids that either is identical or that represent conservative amino acid substitutions.

### DNA Molecules of the Invention

The present invention also relates to the DNA molecules that encode an antibody of the invention or antigen-binding fragment thereof. The DNA sequences used for the antibodies expressed are given in Table 32. These sequences are optimized for mammalian expression. DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):
a. Tₘ=69.3+0.41(% G+C)° C.
b. The Tₘ of a duplex DNA decreases by 1° C. with every increase of 1% in the number of mismatched base pairs.
c. (Tₘ)_{μ2}−(Tₘ)_{μ1}=18.5 log₁₀μ2/μ1
   where μ1 and μ2 are the ionic strengths of two solutions.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

### Functionally Equivalent Variants

Yet another class of DNA variants within the scope of the invention may be described with reference to the product they encode. These functionally equivalent polynucleotides are characterized by the fact that they encode the same peptide sequences due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences of the present invention (see Table 32). The recombinant constructs of the present invention are used in connection with a vector, such as a plasmid, phagemid, phage or viral vector, into which a DNA molecule encoding an antibody of the invention or antigen-binding fragment thereof or variant thereof is inserted.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

An antibody, antigen binding portion, or variant thereof provided herein can be prepared by recombinant expression of nucleic acid sequences encoding light and heavy chains or portions thereof in a host cell. To express an antibody, antigen binding portion, or variant thereof recombinantly, a host cell can be transfected with one or more recombinant expression vectors carrying DNA fragments encoding the light and/or heavy chains or portions thereof such that the light and heavy chains are expressed in the host cell. Standard recombinant DNA methodologies are used to prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., el al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

In certain assays an expression of the antibodies of this invention as murine IgG is preferred, e.g. immunohistochemistry with human samples can be analyzed more easily by using murine antibodies.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding fragments thereof or variants thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic host cells are yeast, insect or mammalian cells. In some embodiments, the DNAs encoding the heavy and light chains are inserted into separate vectors. In other embodiments, the DNA encoding the heavy and light chains is inserted into the same vector. It is understood that the design of the expression vector, including the selection of regulatory sequences is affected by factors such as the choice of the host cell, the level of expression of protein desired and whether expression is constitutive or inducible.

### Bacterial Expression

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include but are not limited to E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and a bacterial origin of replication derived from commercially available plasmids typically containing elements of the well-known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable.

Therefore, an embodiment of the present invention is an expression vector comprising a nucleic acid sequence encoding for the novel antibodies of the present invention. See Example 1 for an exemplary description.

Antibodies of the present invention or antigen-binding fragments thereof or variants thereof include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression & Purification

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and U.S. Pat. No. 5,179,017, by Axel et al.). Suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418.

Transfection of the expression vector into a host cell can be carried out using standard techniques such as electroporation, calcium-phosphate precipitation, and DEAE-dextran transfection.

Suitable mammalian host cells for expressing the antibodies, antigen binding fragments thereof or variants thereof provided herein include Chinese Hamster Ovary (CHO cells) [including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621], NSO myeloma cells, COS cells and SP2 cells. In some embodiments, the expression vector is designed such that the expressed protein is secreted into the culture medium in which the host cells are grown. The antibodies, antigen binding fragments thereof or variants thereof can be recovered from the culture medium using standard protein purification methods.

Antibodies of the invention or antigen-binding fragments thereof or variants thereof can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof or variants thereof include naturally purified products, products of chemical synthetic procedures, and products produced by recombinant techniques from an eukaryotic host, including, for example, yeast, higher plant, insect and mammalian cells. Depending upon the host employed in a recombinant production procedure, the antibody of the present invention can be glycosylated or can be non-glycosylated. Such methods are described in many standard laboratory manuals, such as Sambrook, supra, Sections 17.37-17.42; Ausubel, supra, Chapters 10, 12, 13, 16, 18 and 20.

Therefore, an embodiment of the present invention are also host cells comprising the vector or a nucleic acid molecule, whereby the host cell can be a higher eukaryotic host cell, such as a mammalian cell, a lower eukaryotic host cell, such as a yeast cell, and may be a prokaryotic cell, such as a bacterial cell.

Another embodiment of the present invention is a method of using the host cell to produce an antibody and antigen binding fragments, comprising culturing the host cell under suitable conditions and recovering said antibody.

Therefore another embodiment of the present invention is the production of the antibodies according to this invention with the host cells of the present invention and purification of these antibodies to at least 95% homogeneity by weight.

### Therapeutic Methods

Therapeutic methods involve administering to a subject in need of treatment a therapeutically effective amount of an antibody or an antigen-binding fragment thereof or a variant thereof contemplated by the invention. A “therapeutically effective” amount hereby is defined as the amount of an antibody or antigen-binding fragment that is of sufficient quantity to reduce proliferation of TWEAKR positive cell or to reduce size of a TWEAKR expressing tumor in a treated area of a subject—either as a single dose or according to a multiple dose regimen, alone or in combination with other agents, which leads to the alleviation of an adverse condition, yet which amount is toxicologically tolerable. The subject may be a human or non-human animal (e.g., rabbit, rat, mouse, dog, monkey or other lower-order primate).

It is an embodiment of the invention to provide antibodies, or antigen-binding antibody fragments thereof, or variants thereof, which have a strong anti-tumor efficacy in a broad panel of cell line-derived and patient-derived human tumor models. Tumor models include but are not limited to 786-O, A375, A253, SK-OV-3, WiDr, SW480, Co5682, NCI-H1975, NCI-H322, Lu7343 and Lu7433 (see Example 8 for further details), Co5676 and Co 5841 (see Example 10 for further details), SCaBER (see Example 11 for further details) and SCC4 (see Example 12 for further details). For example a dose-dependent efficacy of TPP-2084 and TPP-2090 is shown in FIG. 19 for the human renal cell cancer model 786-O. In vivo anti-tumor efficacy is shown exemplary for TPP-2090 in human colon cancer xenograft WiDr in FIG. 20 and in human lung cancer xenograft NCI-H322 in FIG. 21. Efficacy of the anti-TWEAKR antibody TPP-2090 was also investigated in other colorectal tumor model such as SW480 and patient-derived tumor model Co5682 in monotherapy and/or combination therapy with similar good results (see Table 29). Further tumor models 786-O, A375, A253, SK-OV-3, Bx-PC3 are shown in Table 28 and NCI-H322, NCI-H1975, Lu7343, and Lu7433 in Table 30.

It is an embodiment of the invention to provide an antibody of the invention or antigen-binding fragment thereof for use as medicament.

It is an embodiment of the invention to provide an antibody of the invention or antigen-binding fragment thereof for use as a medicament for the treatment of cancer. In a preferred embodiment the cancer is a solid tumor. It is an embodiment of the invention to provide an antibody of the invention or an antigen-binding fragment thereof for use in the treatment of cancer. In a preferred embodiment the cancer is a solid tumor.

It is an embodiment of the invention to use an antibody of the invention or an antigen-binding fragment thereof for the manufacture of a medicament for use in the treatment of cancer. In a preferred embodiment the cancer is a solid tumor.

It is another embodiment of the invention to provide a method for the treatment of cancer comprising administering a therapeutically effective amount of an antibody of the invention or an antigen-binding fragment thereof to a subject in need thereof. In a preferred embodiment the cancer is a solid tumor.

An antibody of the invention or an antigen-binding fragment thereof or a variant thereof might be co-administered with known medicaments, and in some instances the antibody might itself be modified. For example, an antibody or an antigen-binding fragment thereof or a variant thereof could be conjugated to a cytotoxic agent or radioisotope to potentially further increase efficacy.

Antibodies of the present invention or antigen-binding fragments thereof or variants thereof may be administered as the sole pharmaceutical agent or in combination with one or more additional therapeutic agents where the combination causes no unacceptable adverse effects. This combination therapy includes administration of a single pharmaceutical dosage formulation which contains an antibody of the invention or an antigen-binding fragment thereof or a variants thereof and one or more additional therapeutic agents, as well as administration of an antibody of the invention and each additional therapeutic agent in its own separate pharmaceutical dosage formulation. For example, an antibody of the invention or an antigen-binding fragment thereof or a variant thereof and a therapeutic agent may be administered to the patient together in a single liquid composition, or each agent may be administered in separate dosage formulation.

Where separate dosage formulations are used, an antibody of the invention or an antigen-binding fragment thereof or a variants thereof and one or more additional therapeutic agents may be administered at essentially the same time (e.g., concurrently) or at separately staggered times (e.g., sequentially).

It is another embodiment of the invention to provide antibodies, or antigen-binding antibody fragments thereof or variants thereof, which have synergistic or additive efficacy in cell line-derived and patient-derived human tumor models if antibody treatment is combined with Irinotecan, Regorafenib, Paclitaxel, PI3K-inhibitor 1, Oxaliplatin, Cisplatin, 5-Fluoruracil (5-FU), Gemcitabine, or Cetuximab.

A preferred embodiment of the invention is a combination of an antibody of the invention, or antigen-binding antibody fragments thereof or variants thereof, with a further active ingredient comprised in the group of ingredients consisting of Irinotecan, Cisplatin, Oxaliplatin, 5-Fluoruracil (5-FU), Regorafenib, and Cetuximab. Even more preferred is a combination of antibody TPP-2090 with a further active ingredient comprised in the group of ingredients consisting of Irinotecan, Cisplatin, 5-Fluoruracil (5-FU) and Regorafenib.

A preferred embodiment of the invention is a combination of an antibody of the invention, or antigen-binding antibody fragments thereof or variants thereof, with a further active ingredient comprised in the group of ingredients consisting of Irinotecan, Oxaliplatin, 5-Fluoruracil (5-FU), Regorafenib, and Cetuximab for use in the treatment of colorectal cancer. Even more preferred is a combination of antibody TPP-2090 with a further active ingredient comprised in the group of ingredients consisting of Irinotecan, 5-Fluoruracil (5-FU) and Regorafenib for use in the treatment of colorectal cancer.

In a preferred embodiment colorectal cancer is treated with a combination of an antibody of the invention, or antigen-binding antibody fragments thereof or variants thereof, with Irinotecan, Oxaliplatin, 5-Fluoruracil (5-FU), Regorafenib, or Cetuximab. Even more preferred is the treatment of colorectal cancer with TPP-2090 in combination with Irinotecan, 5-Fluoruracil (5-FU) or Regorafenib.

A further preferred embodiment is a combination of an antibody of the invention, or an antigen-binding antibody fragment thereof or variants thereof, with Cisplatin for use in the treatment of bladder cancer. Even more preferred a combination of antibody TPP-2090 with Cisplatin for use in the treatment of bladder cancer.

A further preferred embodiment is the treatment of bladder cancer with a combination of an antibody of the invention, or an antigen-binding antibody fragment thereof or variants thereof, with Cisplatin. Even more preferred is the treatment of bladder cancer with TPP-2090 in combination with Cisplatin.

In the human colon cancer xenograft WiDr a clear positive effect can be demonstrated if e.g. TPP-2090 is combined with Irinotecan or Regorafenib. In the human lung cancer xenografts NCI-H322 and NCI-H1975 a positive effect can be demonstrated if e.g. TPP-2090 is combined with Paclitaxel.

In particular, antibodies of the present invention or antigen-binding fragments thereof or variants thereof may be used in fixed or separate combination with other anti-tumor agents such as alkylating agents, anti-metabolites, plant-derived anti-tumor agents, hormonal therapy agents, topoisomerase inhibitors, camptothecin derivatives, kinase inhibitors, targeted drugs, antibodies, interferons and/or biological response modifiers, anti-angiogenic compounds, and other anti-tumor drugs. In this regard, the following is a non-limiting list of examples of secondary agents that may be used in combination with the antibodies of the present invention:

Alkylating agents include, but are not limited to, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, bendamustin, and mitolactol; platinum-coordinated alkylating compounds include, but are not limited to, cisplatin, carboplatin, eptaplatin, lobaplatin, nedaplatin, oxaliplatin, and satraplatin;

Anti-metabolites include, but are not limited to, methotrexate, 6-mercaptopurine riboside, mercaptopurine, 5-fluorouracil alone or in combination with leucovorin, tegafur, doxifluridine, carmofur, cytarabine, cytarabine ocfosfate, enocitabine, gemcitabine, fludarabin, 5-azacitidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine;

Hormonal therapy agents include, but are not limited to, exemestane, Lupron, anastrozole, doxercalciferol, fadrozole, formestane, 11-beta hydroxysteroid dehydrogenase 1 inhibitors, 17-alpha hydroxylase/17,20 lyase inhibitors such as abiraterone acetate, 5-alpha reductase inhibitors such as finasteride and epristeride, anti-estrogens such as tamoxifen citrate and fulvestrant, Trelstar, toremifene, raloxifene, lasofoxifene, letrozole, anti-androgens such as bicalutamide, flutamide, mifepristone, nilutamide, Casodex, and anti-progesterones and combinations thereof;

Plant-derived anti-tumor substances include, e.g., those selected from mitotic inhibitors, for example epothilones such as sagopilone, ixabepilone and epothilone B, vinblastine, vinflunine, docetaxel, and Paclitaxel;

Cytotoxic topoisomerase inhibiting agents include, but are not limited to, aclarubicin, doxorubicin, amonafide, belotecan, camptothecin, 10-hydroxycamptothecin, 9-aminocamptothecin, diflomotecan, Irinotecan, topotecan, edotecarin, epimbicin, etoposide, exatecan, gimatecan, lurtotecan, mitoxantrone, pirambicin, pixantrone, rubitecan, sobuzoxane, tafluposide, and combinations thereof;

Immunologicals include interferons such as interferon alpha, interferon alpha-2a, interferon alpha-2b, interferon beta, interferon gamma-1a and interferon gamma-n1, and other immune enhancing agents such as L19-IL2 and other IL2 derivatives, filgrastim, lentinan, sizofilan, TheraCys, ubenimex, aldesleukin, alemtuzumab, BAM-002, dacarbazine, daclizumab, denileukin, gemtuzumab, ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, and Provenge;

Biological response modifiers are agents that modify defense mechanisms of living organisms or biological responses such as survival, growth or differentiation of tissue cells to direct them to have anti-tumor activity; such agents include, e.g., krestin, lentinan, sizofiran, picibanil, ProMune, and ubenimex;

Anti-angiogenic compounds include, but are not limited to, acitretin, aflibercept, angiostatin, aplidine, asentar, axitinib, bevacizumab, brivanib alaninat, cilengtide, combretastatin, endostatin, fenretinide, halofuginone, pazopanib, ranibizumab, rebima-stat, recentin, regorafenib, removab, revlimid, sorafenib, squalamine, sunitinib, telatinib, thalidomide, ukrain, vatalanib, and vitaxin;

Antibodies include, but are not limited to, trastuzumab, cetuximab, bevacizumab, rituximab, ticilimumab, ipilimumab, lumiliximab, catumaxomab, atacicept, oregovomab, panitumumab and alemtuzumab;

VEGF inhibitors such as, e.g., sorafenib, regorafenib, bevacizumab, sunitinib, recentin, axitinib, aflibercept, telatinib, brivanib alaninate, vatalanib, pazopanib, and ranibizumab;

EGFR (HER1) inhibitors such as, e.g., cetuximab, panitumumab, vectibix, gefitinib, erlotinib, and Zactima;

HER2 inhibitors such as, e.g., lapatinib, tratuzumab, and pertuzumab;

mTOR inhibitors such as, e.g., temsirolimus, sirolimus/Rapamycin, and everolimus;

c-Met inhibitors;

PI3K inhibitors such as PI3K inhibitor 1 (2-amino-N-[7-methoxy-8-(3-morpholin-4-ylpropoxy)-2,3-dihydroimidazo[1,2-c]quinazolin-5-yl]pyrimidine-5-carboxamide dihydrochloride (see compound of Examples 1 and 2 WO 2012/136553, (which is incorporated herein by reference in its entirety)

and AKT inhibitors;

CDK inhibitors such as roscovitine and flavopiridol;

Spindle assembly checkpoints inhibitors and targeted anti-mitotic agents such as PLK inhibitors, Aurora inhibitors (e.g. Hesperadin), checkpoint kinase inhibitors, and KSP inhibitors;

HDAC inhibitors such as, e.g., panobinostat, vorinostat, MS275, belinostat, and LBH589;

HSP90 and HSP70 inhibitors;

Proteasome inhibitors such as bortezomib and carfilzomib;

Serine/threonine kinase inhibitors including MEK inhibitors and Raf inhibitors such as sorafenib;

Farnesyl transferase inhibitors such as, e.g., tipifarnib;

Tyrosine kinase inhibitors including, e.g., dasatinib, nilotibib, regorafenib, bosutinib, sorafenib, bevacizumab, sunitinib, cediranib, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab, and c-Kit inhibitors;

Vitamin D receptor agonists;

Bcl-2 protein inhibitors such as obatoclax, oblimersen sodium, and gossypol;

Cluster of differentiation 20 receptor antagonists such as, e.g., rituximab;

Ribonucleotide reductase inhibitors such as, e.g., gemcitabine;

Tumor necrosis factor related apoptosis inducing ligand receptor 1 agonists such as, e.g., mapatumumab;

Tumor necrosis factor related apoptosis inducing ligand receptor 2 agonists such as e.g., lexatumumab, conatumumab, CS-1008, PRO95780;

5-Hydroxytryptamine receptor antagonists such as, e.g., rEV598, xaliprode, palonosetron hydrochloride, granisetron, Zindol, and AB-1001;

Integrin inhibitors including alpha5-beta1 integrin inhibitors such as, e.g., E7820, JSM 6425, volociximab, and endostatin;

Androgen receptor antagonists including, e.g., nandrolone decanoate, fluoxymesterone, Android, Prost-aid, andromustine, bicalutamide, flutamide, apo-cyproterone, apo-flutamide, chlormadinone acetate, Androcur, Tabi, cyproterone acetate, and nilutamide;

Aromatase inhibitors such as, e.g., anastrozole, letrozole, testolactone, exemestane, aminoglutethimide, and formestane;

Matrix metalloproteinase inhibitors;

Other anti-cancer agents including, e.g., alitretinoin, ampligen, atrasentan bexarotene, bortezomib, bosentan, calcitriol, exisulind, fotemustine, ibandronic acid, miltefosine, mitoxantrone, I-asparaginase, procarbazine, dacarbazine, hydroxycarbamide, pegaspargase, pentostatin, tazaroten, velcade, gallium nitrate, canfosfamide, darinaparsin, and tretinoin.

In a preferred embodiment, the antibodies of the present invention may be used in combination with chemotherapy (i.e. cytotoxic agents), anti-hormones and/or targeted therapies such as other kinase inhibitors (for example, EGFR inhibitors), mTOR inhibitors and angiogenesis inhibitors.

The compounds of the present invention may also be employed in cancer treatment in conjunction with radiation therapy and/or surgical intervention.

An antibody of the invention or antigen-binding fragment thereof might in some instances itself be modified. For example, an antibody could be conjugated to any of but not limited to the compounds mentioned above or any radioisotope to potentially further increase efficacy. Furthermore, the antibodies of the invention may be utilized, as such or in compositions, in research and diagnostics, or as analytical reference standards, and the like, which are well known in the art.

The inventive antibodies or antigen-binding fragments thereof can be used as a therapeutic or a diagnostic tool in a variety of situations with aberrant TWEAKR-signaling, e.g. cell proliferative disorders such as cancer or fibrotic diseases. Disorders and conditions particularly suitable for treatment with an antibody of the inventions are solid tumors, such as cancers of the breast, respiratory tract, brain, reproductive organs, digestive tract, urinary tract, eye, liver, skin, head and neck, thyroid, parathyroid, and their distant metastases. Those disorders also include lymphomas, sarcomas and leukemias.

Tumors of the digestive tract include, but are not limited to anal, colon, colorectal, esophageal, gallbladder, gastric, pancreatic, rectal, small-intestine, and salivary gland cancers.

Examples of esophageal cancer include, but are not limited to esophageal cell carcinomas and Adenocarcinomas, as well as squamous cell carcinomas, Leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to ductal adenocarcinoma, adenosquamous carcinomas and pancreatic endocrine tumors.

Examples of breast cancer include, but are not limited to triple negative breast cancer, invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal and vulvar cancer, as well as sarcoma of the uterus.

Examples of ovarian cancer include, but are not limited to serous tumour, endometrioid tumor, mucinous cystadenocarcinoma, granulosa cell tumor, Sertoli-Leydig cell tumor and arrhenoblastoma

Examples of cervical cancer include, but are not limited to squamous cell carcinoma, adenocarcinoma, adenosquamous carcinoma, small cell carcinoma, neuroendocrine tumour, glassy cell carcinoma and villoglandular adenocarcinoma.

Tumors of the urinary tract include, but are not limited to bladder, penile, kidney, renal pelvis, ureter, urethral, and hereditary and sporadic papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to transitional cell carcinoma, squamous cell carcinoma, adenocarcinoma, sarcoma and small cell carcinoma.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to squamous cell cancer of the head and neck, laryngeal, hypopharyngeal, nasopharyngeal, oropharyngeal cancer, salivary gland cancer, lip and oral cavity cancer, and squamous cell cancer.

Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

In a preferred embodiment, the antibodies or antigen-binding fragments thereof of the invention are suitable for a therapeutic or diagnostic method for the treatment or diagnosis of a cancer disease. In a preferred embodiment, the antibodies or antigen-binding fragments thereof of the invention are suitable for a therapeutic or diagnostic method for the treatment or diagnosis of a cancer disease wherein the cancer is a solid cancer.

In a preferred embodiment, the antibodies of the invention or antigen-binding fragments thereof are suitable for a therapeutic or diagnostic method for the treatment or diagnosis of a cancer disease comprised in a group consisting of gastric cancer, breast cancer, pancreatic cancer, colorectal cancer, kidney cancer, prostate cancer, ovarian cancer, cervical cancers, lung cancer, endometrial cancer, esophageal cancer, head and neck cancer, hepatocellular carcinoma, melanoma and bladder cancer.

In a more preferred embodiment, the antibodies of the invention or antigen-binding fragments thereof are suitable for a therapeutic or diagnostic method for the treatment or diagnosis of a cancer disease comprised in a group consisting of bladder cancer, colorectal cancer, non small cell lung cancer, kidney cancer, melanoma, ovarian cancer, head and neck cancer and pancreatic cancer.

In a more preferred embodiment, the antibodies of the invention or antigen-binding fragments thereof are for use in a therapeutic method for the treatment of a cancer disease comprised in a group consisting of bladder cancer, colorectal cancer, non small cell lung cancer, kidney cancer, melanoma, ovarian cancer, head and neck cancer and pancreatic cancer.

A more preferred embodiment is the use of the antibodies of the invention or antigen-binding fragments thereof for the manufacture of a medicament for use in the treatment of a cancer disease comprised in a group consisting of bladder cancer, colorectal cancer, non small cell lung cancer, kidney cancer, melanoma, ovarian cancer, head and neck cancer and pancreatic cancer.

A more preferred embodiment, is a method for the treatment of a cancer disease comprised in a group consisting of bladder cancer, colorectal cancer, non small cell lung cancer, kidney cancer, melanoma, ovarian cancer, head and neck cancer and pancreatic cancer, comprising the administration of a therapeutic effective amount of the antibodies of the invention or antigen-binding fragments.

In addition, the inventive antibodies or antigen-binding fragments thereof can also be used as a therapeutic or a diagnostic tool in a variety of other disorders wherein TWEAKR is involved such as, but not limited to fibrotic diseases such as intraalveolar fibrosis, silica-induced pulmonary fibrosis, experimental lung fibrosis, idiopathic lung fibrosis, renal fibrosis, as well as lymphangioleiomyomatosis, polycystic ovary syndrome, acne, psoriasis, cholesteatoma, cholesteatomatous chronic otitis media, periodontitis, solar lentigines, bowel disease, atherosclerosis or endometriosis.

The disorders mentioned above have been well characterized in humans, but also exist with a similar etiology in other animals, including mammals, and can be treated by administering pharmaceutical compositions of the present invention.

To treat any of the foregoing disorders, pharmaceutical compositions for use in accordance with the present invention may be formulated in a conventional manner using one or more physiologically acceptable carriers or excipients. An antibody of the invention or antigen-binding fragment thereof can be administered by any suitable means, which can vary, depending on the type of disorder being treated. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the invention or an antigen-binding fragment thereof or variants thereof might be administered by pulse infusion, with, e.g., declining doses of the antibody. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

Determining a therapeutically effective amount of the novel antibody of this invention or an antigen-binding fragment thereof or a variant thereof, largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

### Diagnostic Methods

Anti-TWEAKR antibodies or antigen-binding fragments thereof can be used for detecting the presence of TWEAKR-expressing tumors. The presence of TWEAKR-containing cells or shed TWEAKR within various biological samples, including serum, and tissue biopsy specimens, may be detected with anti-TWEAKR antibodies. In addition, anti-TWEAKR antibodies may be used in various imaging methodologies such as immunoscintigraphy with a ⁹⁹Tc (or other isotope) conjugated antibody. For example, an imaging protocol similar to the one described using a ¹¹¹In conjugated anti-PSMA antibody may be used to detect pancreatic or ovarian carcinomas (Sodee et al., Clin. Nuc. Med. 21: 759-766, 1997). Another method of detection that can be used is positron emitting tomography by conjugating the antibodies of the invention with a suitable isotope (see Herzog et al., J. Nucl. Med. 34:2222-2226, 1993).

### Pharmaceutical Compositions and Administration

An embodiment of the present invention are pharmaceutical compositions which comprise anti-TWEAKR antibodies or antigen-binding fragments thereof or variants thereof, alone or in combination with at least one other agent, such as a stabilizing compound, which may be administered in any sterile, biocompatible pharmaceutical carrier, including, but not limited to, saline, buffered saline, dextrose, and water. A further embodiment are pharmaceutical compositions comprising a TWEAKR binding antibody or antigen-binding fragment thereof and a further pharmaceutically active compound that is suitable to treat TWEAKR related diseases such as cancer. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial (directly to the tumor), intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

The term “pharmaceutical formulation” refers to a preparation which is in such form as to permit the biological activity of an active ingredient contained therein to be effective, and which contains no additional components which are unacceptably toxic to a subject to which the formulation would be administered.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl-cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores can be provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

### Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

In another embodiment, the kits may contain DNA sequences encoding the antibodies of the invention or antigen-binding fragments thereof or variants thereof. Preferably the DNA sequences encoding these antibodies are provided in a plasmid suitable for transfection into and expression by a host cell. The plasmid may contain a promoter (often an inducible promoter) to regulate expression of the DNA in the host cell. The plasmid may also contain appropriate restriction sites to facilitate the insertion of other DNA sequences into the plasmid to produce various antibodies. The plasmids may also contain numerous other elements to facilitate cloning and expression of the encoded proteins. Such elements are well known to those of skill in the art and include, for example, selectable markers, initiation codons, termination codons, and the like.

### Manufacture and Storage.

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition. For administration of anti-TWEAKR antibodies or antigen-binding fragment thereof, such labeling would include amount, frequency and method of administration.

### Therapeutically Effective Dose.

Pharmaceutical compositions suitable for use in the present invention include compositions wherein the active ingredients are contained in an effective amount to achieve the intended purpose, i.e. treatment of a particular disease state characterized by TWEAKR expression. The determination of an effective dose is well within the capability of those skilled in the art.

For any compound, the therapeutically effective dose can be estimated initially either in cell culture assays, e.g., neoplastic cells, or in animal models, usually mice, rabbits, dogs, pigs or monkeys. The animal model is also used to achieve a desirable concentration range and route of administration. Such information can then be used to determine useful doses and routes for administration in humans.

A therapeutically effective dose refers to that amount of antibody or antigen-binding fragment thereof, that ameliorate the symptoms or condition. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from cell culture assays and animal studies are used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors that may be taken into account include the severity of the disease state, e.g., tumor size and location; age, weight and gender of the patient; diet, time and frequency of administration, drug combination(s), reaction sensitivities, and tolerance/response to therapy. Long acting pharmaceutical compositions might be administered for example every 3 to 4 days, every week, once every two weeks, or once every three weeks, depending on half-life and clearance rate of the particular formulation.

Normal dosage amounts may vary from 0.1 to 100,000 micrograms, up to a total dose of about 2 g, depending upon the route of administration. Guidance as to particular dosages and methods of delivery is provided in the literature. See U.S. Pat. Nos. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc. Preferred specific activities for a radiolabelled antibody may range from 0.1 to 10 mCi/mg of protein (Riva et al., Clin. Cancer Res. 5:3275-3280, 1999; Ulaner et al., 2008 Radiology 246(3):895-902)

A further preferred embodiment of the invention is:
1. An isolated anti-TWEAKR antibody or an antigen-binding fragment thereof, which specifically binds to the D at position 47 (D47) of TWEAKR (SEQ ID NO:169).
2. The antibody or an antigen binding fragment thereof according to embodiment 1 wherein the antibody specifically binds to the D at position 47 (D47) of TWEAKR (SEQ ID NO:169), when the antibody loses more than 80% of its ELISA signal on TPP-2614 compared to TPP-2203
3. The antibody or an antigen binding fragment thereof according to embodiment 1 or 2 wherein the antibody is an agonistic antibody.
4. The antibody or an antigen binding fragment thereof according to anyone of the preceding embodiments, which comprises:
   a variable heavy chain comprising:
   (a) a heavy chain CDR1 encoded by an amino acid sequence comprising the formula PYPMX (SEQ ID NO: 171), wherein X is I or M;
   (b) a heavy chain CDR2 encoded by an amino acid sequence comprising the formula YISPSGGXTHYADSVKG (SEQ ID NO: 172), wherein X is S or K; and
   (c) a heavy chain CDR3 encoded by an amino acid sequence comprising the formula GGDTYFDYFDY (SEQ ID NO: 173);
   and a variable light chain comprising:
   (a) a light chain CDR1 encoded by an amino acid sequence comprising the formula RASQSISXYLN (SEQ ID NO: 174), wherein X is G or S;
   (b) a light chain CDR2 encoded by an amino acid sequence comprising the formula XASSLQS (SEQ ID NO: 175), wherein X is Q, A, or N; and
   (c) a light chain CDR3 encoded by an amino acid sequence comprising the formula QQSYXXPXIT (SEQ ID NO: 176), wherein X at position 5 is T or S, and X at position 6 is T or S, and X at position 8 is G, or F.
5. The antibody or an antigen binding fragment thereof according to anyone of the preceding embodiments comprising:
   a. a variable heavy chain comprising the variable heavy chain CDR1 sequence as presented by SEQ ID NO: 6, the variable heavy chain CDR2 sequence as presented by SEQ ID NO: 7, and the variable heavy chain CDR3 sequence as presented by SEQ ID NO: 8, and a variable light chain comprising the variable light chain CDR1 sequence presented by SEQ ID NO: 3, the variable light chain CDR2 sequence presented by SEQ ID NO: 4, and the variable light chain CDR3 sequence presented by SEQ ID NO: 5, or
   b. a variable heavy chain comprising the variable heavy chain CDR1 sequence as presented by SEQ ID NO: 16, the variable heavy chain CDR2 sequence as presented by SEQ ID NO: 17, the variable heavy chain CDR3 sequence as presented by SEQ ID NO:18, and
      a variable light chain comprising the variable light chain CDR1 sequence presented by SEQ ID NO: 13, the variable light chain CDR2 sequence presented by SEQ ID NO: 14, and the variable light chain CDR3 sequence presented by SEQ ID NO:15, or
   c. a variable heavy chain comprising the variable heavy chain CDR1 sequence as presented by SEQ ID NO: 26, the variable heavy chain CDR2 sequence as presented by SEQ ID NO: 27, the variable heavy chain CDR3 sequence as presented by SEQ ID NO:28, and
      a variable light chain comprising the variable light chain CDR1 sequence presented by SEQ ID NO: 23, the variable light chain CDR2 sequence presented by SEQ ID NO: 24, and the variable light chain CDR3 sequence presented by SEQ ID NO:25, or
   d. a variable heavy chain comprising the variable heavy chain CDR1 sequence as presented by SEQ ID NO: 36, the variable heavy chain CDR2 sequence as presented by SEQ ID NO: 37, the variable heavy chain CDR3 sequence as presented by SEQ ID NO:38, and
      a variable light chain comprising the variable light chain CDR1 sequence presented by SEQ ID NO: 33, the variable light chain CDR2 sequence presented by SEQ ID NO: 34, and the variable light chain CDR3 sequence presented by SEQ ID NO:35, or
   e. a variable heavy chain comprising the variable heavy chain CDR1 sequence as presented by SEQ ID NO: 46, the variable heavy chain CDR2 sequence as presented by SEQ ID NO: 47, the variable heavy chain CDR3 sequence as presented by SEQ ID NO:48, and
      a variable light chain comprising the variable light chain CDR1 sequence presented by SEQ ID NO: 43, the variable light chain CDR2 sequence presented by SEQ ID NO: 44, and the variable light chain CDR3 sequence presented by SEQ ID NO:45, or
   f. a variable heavy chain comprising the variable heavy chain CDR1 sequence as presented by SEQ ID NO: 56, the variable heavy chain CDR2 sequence as presented by SEQ ID NO: 57, the variable heavy chain CDR3 sequence as presented by SEQ ID NO:58, and
      a variable light chain comprising the variable light chain CDR1 sequence presented by SEQ ID NO: 53, the variable light chain CDR2 sequence presented by SEQ ID NO: 54, and the variable light chain CDR3 sequence presented by SEQ ID NO:55, or
   g. a variable heavy chain comprising the variable heavy chain CDR1 sequence as presented by SEQ ID NO: 66, the variable heavy chain CDR2 sequence as presented by SEQ ID NO: 67, the variable heavy chain CDR3 sequence as presented by SEQ ID NO:68, and
      a variable light chain comprising the variable light chain CDR1 sequence presented by SEQ ID NO: 63, the variable light chain CDR2 sequence presented by SEQ ID NO: 64, and the variable light chain CDR3 sequence presented by SEQ ID NO:65, or
   h. a variable heavy chain comprising the variable heavy chain CDR1 sequence as presented by SEQ ID NO: 76, the variable heavy chain CDR2 sequence as presented by SEQ ID NO: 77, the variable heavy chain CDR3 sequence as presented by SEQ ID NO:78, and
      a variable light chain comprising the variable light chain CDR1 sequence presented by SEQ ID NO: 73, the variable light chain CDR2 sequence presented by SEQ ID NO: 74, and the variable light chain CDR3 sequence presented by SEQ ID NO:75, or
   i. a variable heavy chain comprising the variable heavy chain CDR1 sequence as presented by SEQ ID NO: 86, the variable heavy chain CDR2 sequence as presented by SEQ ID NO: 87, the variable heavy chain CDR3 sequence as presented by SEQ ID NO:88, and
      a variable light chain comprising the variable light chain CDR1 sequence presented by SEQ ID NO: 83, the variable light chain CDR2 sequence presented by SEQ ID NO: 84, and the variable light chain CDR3 sequence presented by SEQ ID NO:85, or
   j. a variable heavy chain comprising the variable heavy chain CDR1 sequence as presented by SEQ ID NO: 96, the variable heavy chain CDR2 sequence as presented by SEQ ID NO: 97, the variable heavy chain CDR3 sequence as presented by SEQ ID NO:98, and
      a variable light chain comprising the variable light chain CDR1 sequence presented by SEQ ID NO: 93, the variable light chain CDR2 sequence presented by SEQ ID NO: 94, and the variable light chain CDR3 sequence presented by SEQ ID NO:95, or
   k. a variable heavy chain comprising the variable heavy chain CDR1 sequence as presented by SEQ ID NO: 106, the variable heavy chain CDR2 sequence as presented by SEQ ID NO: 107, the variable heavy chain CDR3 sequence as presented by SEQ ID NO:108, and
      a variable light chain comprising the variable light chain CDR1 sequence presented by SEQ ID NO: 103, the variable light chain CDR2 sequence presented by SEQ ID NO: 104, and the variable light chain CDR3 sequence presented by SEQ ID NO:105 or
   l. a variable heavy chain comprising the variable heavy chain CDR1 sequence as presented by SEQ ID NO: 116, the variable heavy chain CDR2 sequence as presented by SEQ ID NO: 117, the variable heavy chain CDR3 sequence as presented by SEQ ID NO:118, and
      a variable light chain comprising the variable light chain CDR1 sequence presented by SEQ ID NO: 113, the variable light chain CDR2 sequence presented by SEQ ID NO: 114, and the variable light chain CDR3 sequence presented by SEQ ID NO:115.
6. The antibody or antigen-binding fragment thereof according to anyone of the preceding embodiments comprising:
   a. a variable heavy chain sequence as presented by SEQ ID NO:10 and a variable light chain sequences as presented by SEQ ID NO:9, or
   b. a variable heavy chain sequence as presented by SEQ ID NO:20 and a variable light chain sequences as presented by SEQ ID NO:19, or
   c. a variable heavy chain sequence as presented by SEQ ID NO:30 and a variable light chain sequences as presented by SEQ ID NO:29, or
   d. a variable heavy chain sequence as presented by SEQ ID NO:40 and a variable light chain sequences as presented by SEQ ID NO:39, or
   e. a variable heavy chain sequence as presented by SEQ ID NO:50 and a variable light chain sequences as presented by SEQ ID NO:49, or
   f. a variable heavy chain sequence as presented by SEQ ID NO:60 and a variable light chain sequences as presented by SEQ ID NO:59, or
   g. a variable heavy chain sequence as presented by SEQ ID NO:70 and a variable light chain sequences as presented by SEQ ID NO:69, or
   h. a variable heavy chain sequence as presented by SEQ ID NO:80 and a variable light chain sequences as presented by SEQ ID NO:79, or
   i. a variable heavy chain sequence as presented by SEQ ID NO:90 and a variable light chain sequences as presented by SEQ ID NO:89, or
   j. a variable heavy chain sequence as presented by SEQ ID NO:100 and a variable light chain sequences as presented by SEQ ID NO:99, or
   k. a variable heavy chain sequence as presented by SEQ ID NO:110 and a variable light chain sequences as presented by SEQ ID NO:109, or
   l. a variable heavy chain sequence as presented by SEQ ID NO:120 and a variable light chain sequences as presented by SEQ ID NO:119.
7. The antibody according to any one of the preceding embodiments, which is an IgG antibody.
8. The antibody according to anyone of the preceding embodiments comprising:
   a. a heavy chain sequence as presented by SEQ ID NO:2 and a light chain sequences as presented by SEQ ID NO:1, or
   b. a heavy chain sequence as presented by SEQ ID NO:12 and a light chain sequences as presented by SEQ ID NO:11, or
   c. a heavy chain sequence as presented by SEQ ID NO:22 and a light chain sequences as presented by SEQ ID NO:21, or
   d. a heavy chain sequence as presented by SEQ ID NO:32 and a light chain sequences as presented by SEQ ID NO:31, or
   e. a heavy chain sequence as presented by SEQ ID NO:42 and a light chain sequences as presented by SEQ ID NO:41, or
   f. a heavy chain sequence as presented by SEQ ID NO:52 and a light chain sequences as presented by SEQ ID NO:51, or
   g. a heavy chain sequence as presented by SEQ ID NO:62 and a light chain sequences as presented by SEQ ID NO:61, or
   h. a heavy chain sequence as presented by SEQ ID NO:72 and a light chain sequences as presented by SEQ ID NO:71, or
   i. a heavy chain sequence as presented by SEQ ID NO:82 and a light chain sequences as presented by SEQ ID NO:81, or
   j. a heavy chain sequence as presented by SEQ ID NO:92 and a light chain sequences as presented by SEQ ID NO:91, or
   k. a heavy chain sequence as presented by SEQ ID NO:102 and a light chain sequences as presented by SEQ ID NO:101, or
   l. a heavy chain sequence as presented by SEQ ID NO:112 and a light chain sequences as presented by SEQ ID NO:111, or
   m. a heavy chain sequence as presented by SEQ ID NO:213 and a light chain sequences as presented by SEQ ID NO:1.
9. The antigen-binding fragment according to any one of the preceding embodiments, which is an scFv, Fab, Fab′ fragment or a F(ab′)2 fragment.
10. The antibody or antigen-binding fragment according to any one of the preceding embodiments, which is a monoclonal antibody or antigen-binding fragment thereof.
11. The antibody or antigen-binding fragment according to any one of the preceding embodiments, which is a human, humanized or chimeric antibody or antigen-binding fragment.
12. An antibody-drug conjugate, comprising an antibody or antigen binding fragment thereof according to embodiments 1 to 11.
13. An isolated nucleic acid sequence that encodes the antibody or antigen-binding fragment according to embodiments 1 to 11.
14. A vector comprising a nucleic acid sequence according to embodiment 13.
15. An isolated cell expressing an antibody or antigen-binding fragment according to any one of the embodiments 1 to 11 and/or comprising a nucleic acid according to embodiment 13 or a vector according to embodiment 14.
16. An isolated cell according to embodiment 15, wherein said cell is a prokaryotic or an eukaryotic cell.
17. A method of producing an antibody or antigen-binding fragment according to any one of the embodiments 1-11 comprising culturing of a cell according to embodiment 16 and purification of the antibody or antigen-binding fragment.
18. An antibody or antigen-binding fragment according to embodiments 1-11 or an antibody-drug conjugate according to embodiment 12 for use as a medicament.
19. An antibody or antigen antigen-binding fragment according to embodiments 1-11 for use as a diagnostic agent.
20. An antibody or antigen-binding fragment according to embodiments 1-11 or an antibody-drug conjugate according to embodiment 12 for use in the treatment of cancer.
21. A pharmaceutical composition comprising an antibody or antigen-binding fragment according to embodiments 1-11 or an antibody-drug conjugate according to embodiment 12.
22. A combination of a pharmaceutical composition according to embodiment 21 and one or more therapeutically active compounds.
23. A method for treating a disorder or condition associated with the undesired presence of TWEAKR, comprising administering to a subject in need thereof an effective amount of the pharmaceutical composition according to embodiment 21 or a combination according to embodiment 22.

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

### EXAMPLE 1

### Antibody Generation from Dyax Antibody Library

A fully human antibody phage display library (Hoet R M et al, Nat Biotechnol 2005; 23(3):344-8) was used to isolate TWEAKR-specific, human monoclonal antibodies of the present invention by protein panning (Hoogenboom H. R., Nat Biotechnol 2005; 23(3):1105-16) with dimeric Fc-fused extracellular domains of human and murine TWEAKR as immobilized target.

TABLE 1
List of recombinant antigens used for antibody selection

| Nomenclature | Description | SEQ ID NO |
|---|---|---|
| TPP-599 | HUMAN-TNFRSF12Aaa28-80-hIgG1-Fc | 138 |
| TPP-601 | MURINE-TNFRSF12Aaa28-80-hIgG1-Fc | 137 |

The antigens were biotinylated using an approximately 2-fold molar excess of biotin-LC-NHS (Pierce; Cat. No. 21347) according to manufacturer's instructions and desalted using Zeba desalting columns (Pierce; Cat. No. 89889). Washed Magnetic beads (Dynabeads) were incubated o/n with 200 nM of biotinylated human antigen at 4° C. and blocked for 1 h at 4° C. with blocking buffer (PBS with 3% BSA, 0.05% Tween-20). The blocked Fab-phage library was added to the blocked TWEAKR-beads (Dynabeads streptavidin M280—Invitrogen 112-06D) and incubated for 30 min at room temperature. After stringent washing (3× in blocking buffer and 9× in PBS (150 mM NaCl; 8 mM Na2HPO4; 1.5 mM KH2PO4; adjusted to pH=7.4-7.6) with 0.05% Tween-20) Fab-phages binding specifically to biotinylated TWEAKR-beads (Dynabeads streptavidin M280—Invitrogen 112-06D) were resuspended in PBS and for amplification directly used for infection of Escherichia coli strain TG1. In selection round two murine TWEAKR (200 nM) was used to select for cross-reactive binders and in selection round three the concentration of human TWEAKR was decreased (100 nM) to augment the selection pressure for high affinity binders.

11 different Fab-phages were identified and the corresponding antibodies were re-cloned into a mammalian IgG expression vector which provides the missing CH2-CH3 domains not present in the soluble Fab. The resulting IgGs were transiently expressed in mammalian cells as described in Tom et al., Chapter 12 in Methods Express: Expression Systems edited by Micheal R. Dyson and Yves Durocher, Scion Publishing Ltd, 2007. Briefly, a CMV-Promoter based expression plasmid was transfected into HEK293-6E cells and incubated in Fernbach—Flasks or Wave-Bags. Expression was at 37° C. for 5 to 6 days in F17 Medium (Invitrogen). 1% Ultra-Low IgG FCS (Invitrogen) and 0.5 mM Valproic acid (Sigma) were supplemented 24 h post transfection. The antibodies were purified by Protein A chromatography and further characterized by their binding affinity to soluble monomeric TWEAKR in ELISA and BIAcore analysis as described in Example 2.

TABLE 2
List of recombinant antigen used for affinity measurement

| | | | Cat. | SEQ |
|---|---|---|---|---|
| | | | No. (Fitzgerald | ID |
| Nomenclature | Description | Origin | Inc) | NO |
| TPP-2305 | hTNFRSF12Aaa28-80 | Human | 30R-AT080 | 168 |

To determine the cell binding characteristics of anti-TWEAKR antibodies, binding was tested by flow cytometry to a panel of cell lines (HT29, HS68, HS578). Cells were suspended in dilutions of the antibodies (5 μg/ml) in FACS buffer, and incubated on ice for 1 h. In the following a secondary antibody (PE goat anti-human IgG, Dianova #109-115-098) was added. After incubation for 1 h on ice cells were analyzed by flow cytometry using a FACS-Array (BD Biosciences).

NF-kappaB reporter gene assays were performed to assess the agonistic activity of all 11 identified antibodies (human IgG1). HEK293 cells were transiently transfected with a NF-kappaB reporter construct (BioCat, cat. No. LR-0051-PA) using 293fectin according to manufacturer's instruction. White poly-lysine coated 384well plates (BD) were seeded with transfected cells in F17 media (serum-free; Invitrogen) at 37C, 5% CO2. On the next day cells were stimulated with purified antibodies at different concentrations for 6 h and subsequently a luciferase assay was carried out following standard procedures.

Internalization is followed by fluorescence labeling of anti-TWEAKR antibodies (CypHer 5E mono NHS ester; GE Healthcare). Prior to treatment HT29 cells (2×10⁴/well) were seeded in 100 μl media in 96-MTP plates (fat, black, clear bottom No 4308776, Applied Biosystems). After 18 h incubation at 37° C./5% CO2 the media (Table No 21) was changed and labeled anti-TWEAKR antibodies were added in different concentrations (10, 5, 2.5, 1, 0.1 μg/ml). The chosen incubation time was 0, 0.25, 0.5, 1, 1.5, 2, 3, 6 and 24 h. Fluorescence measurement was performed with an InCell analyzer 1000 (GE Healthcare).

The antibody with the strongest in vitro efficacy (TPP-883) was selected for further potency and affinity maturation.

Amino acid sequences of the light (SEQ ID NO. 71)
and heavy (SEQ ID NO. 72) chains of TPP-883; CDRs
of both the heavy and light chain are underlined.
TPP-883
SEQ ID NO. 71
AQDIQMTQSPATLSASVGDRVTITCRASQSISSYLNWYQQKPGKAPKL
LIYAASSLQSGVPSRFSGSGSGTDFTLTISSLQPEDFATYYCQQSYSS
PGITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 72
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDGYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEW
ESNGQPENNYKTTPPVLDSDGSFFLYSKLTVDKSRWQQGNVFSCSVMH
EALHNHYTQKSLSLSPG

Maturation was done by a first mutation gathering round followed by recombination of the most affinity- and potency-increasing amino acid changes. For mutation gathering NNK (N=AGCT, K=G or T) randomizations at the following individual amino acid positions were generated by site directed mutagenesis using synthetic oligonucleotides including NNK codon-diversification (continuous amino acid nomenclature, compare FIG. 25): S35, S36, Y37 and N39 in CDR-L1; A51, S53, S54, Q56 and S57 in CDR-L2; S92, Y93, S94, S95, G97 and 198 in CDR-L3; P31, Y32, P33, M34 and M35 in CDR-H1; Y50, S52, P53, S54, G56, K57 and H59 in CDR-H2; G99, G100, D101, G102, Y103, F104, D105 and Y106 in CDR-H3. The DNA of all single NNK saturation mutagenesis libraries were re-cloned in a mammalian IgG expression vector for potency maturation and in a phagemid vector for affinity maturation, respectively. Affinity maturation was done by phage panning Washed magnetic beads (Dynabeads) were incubated o/n with 10 nM, 1 nM, 100 pM and 10 pM of biotinylated human antigen at 4° C. and blocked for 1 h at 4° C. with blocking buffer (PBS with 3% BSA, 0.05% Tween-20). The blocked Fab-phage library was added with 10000-fold, 1000-fold and 100-fold excess compared to the theoretical library complexity to the blocked TWEAKR-Dynabeads and incubated for 30 min at room temperature. Thus in total, 12 strategies were followed (4 antigen concentrations×3 Fab-phage titers). After stringent washing (3× in blocking buffer and 9× in PBS with 0.05% Tween-20) Fab-phages binding specifically to biotinylated TWEAKR-Dynabeads (Dynabeads streptavidin M280—Invitrogen 112-06D) were resuspended in PBS and for amplification directly used for infection of Escherichia coli strain TG1. In selection round two the concentration of human TWEAKR-Fc was decreased (1 nM, 100 pM, 10 pM and 1 pM) and the same Fab-phage titer was used for all 12 strategies (4.4×10″). For soluble Fab expression the phagemid vector was digested with the restriction endonuclease MluI to remove the geneIII membrane anchor sequence required for Fab display on phage and religated. 96 variants of each of the 12 selection pools were expressed as soluble Fabs and tested in an ELISA format. Therefore, 2.5 nM biotinylated TWEAKR-Fc antigen were coated and binding of soluble Fabs was detected by Anti-c-Myc antibody (Abcam ab62928). 7 single substitutions variants (continuous amino acid nomenclature, compare FIG. 25) were detected with improved binding to TWEAKR-Fc (Seq ID No 138): S36G of CDR-L1, A51Q and S57K of CDR-L2, S94T and G97F of CDR-L3, M35I of CDR-H1 and G102T of CDR-H3. For potency maturation HEK293 cells were transfected with an NF-kappaB reporter (BioCat, cat. No. LR-0051-PA). White poly-lysine coated 384well plates (BD) were seeded with transfected cells in F17 media (serum-free; Invitrogen) and individual variants of the NNK-diversified positional antibody (human IgG1) libraries were transiently expressed in mammalian cells. On the next day NF-kappaB reporter cells were stimulated with the expressed single NNK mutagenesis antibody variants for 6 h and subsequently a luciferase assay was carried out following standard procedures. 1 single substitution variant was detected with improved agonistic activity: G102T of CDR-H3. This variant was also obtained from affinity maturation and showed also there the greatest affinity enhancement. After mutation gathering by affinity and potency screening all 7 beneficial single substitutions were recombined (library complexity: 128 variants) in one recombination library. To this end, oligonucleotides were synthesized to introduce selected mutations or the corresponding wild type amino acid at each selected position. Library construction was performed using sequential rounds of overlap extension PCR. The final PCR product was ligated into a bacterial soluble Fab expression vector and 528 variants were randomly selected (˜4fold oversampling) for equilibrium ELISA screening with soluble Fabs as described before. Finally, 7 variants were selected based on enhanced affinity compared to the best single substitution variant, G102T. The corresponding DNA of these were re-cloned in a mammalian IgG expression vector and tested for functional activity in the afore mentioned NF-kappaB reporter cell assay. Finally, the obtained sequences were compared with human germline sequences and deviations without significant impact on affinity and potency were adjusted. Antibodies with the following sequences were obtained by antibody library screening and by affinity and/or potency maturation:

Amino acid sequences of the light (SEQ ID NO. 1)
and heavy (SEQ ID NO. 2) chains of TPP-2090; CDRs
of both the heavy and light chain are underlined.
TPP-2090
SEQ ID NO. 1:
DIQMTQSPSSLSASVGDRVTITCRASQSISGYLNWYQQKPGKAPKLLI
YQASSLQSGVPSRFSGSGSGTDFTLTISSLQPEDFATYYCQQSYTSPF
ITFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC
SEQ ID NO. 2:
EVQLLESGGGLVQPGGSLRLSCAASGFTFSPYPMIWVRQAPGKGLEWV
SYISPSGGSTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 11)
and heavy (SEQ ID NO. 12) chains of TPP-2149;
CDRs of both the heavy and light chain are
underlined.
TPP-2149
SEQ ID NO. 11
DIQMTQSPATLSASVGDRVTITCRASQSISGYLNWYQQKPGKAPKLLI
YQASSLQSGVPSRFSGSGSGTDFTLTISSLQPEDFATYYCQQSYTSPF
ITFGPGTKVEIKRTVAAPSVFIFPPSDEQLKSGTASVVCLLNNFYPRE
AKVQWKVDNALQSGNSQESVTEQDSKDSTYSLSSTLTLSKADYEKHKL
YACEVTHQGLSSPVTKSFNRGEC
SEQ ID NO. 12
EVQLLESGGGLVQPGGSLRLSCAASGFTFSPYPMIWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 21)
and heavy (SEQ ID NO. 22) chains of TPP-2093;
CDRs of both the heavy and light chain are
underlined.
TPP-2093
SEQ ID NO. 21
DIQMTQSPSSLSASVGDRVTITCRASQSISSYLNWYQQKPGKAPKLLI
YQASSLQSGVPSRFSGSGSGTDFTLTISSLQPEDFATYYCQQSYTSPF
ITFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC
SEQ ID NO. 22
EVQLLESGGGLVQPGGSLRLSCAASGFTFSPYPMMWVRQAPGKGLEWV
SYISPSGGSTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 31)
and heavy (SEQ ID NO. 32) chains of TPP-2148;
CDRs of both the heavy and light chain are
underlined.
TPP-2148
SEQ ID NO. 31
DIQMTQSPATLSASVGDRVTITCRASQSISSYLNWYQQKPGKAPKLLI
YQASSLQSGVPSRFSGSGSGTDFTLTISSLQPEDFATYYCQQSYTSPF
ITFGPGTKVEIKRTVAAPSVFIFPPSDEQLKSGTASVVCLLNNFYPRE
AKVQWKVDNALQSGNSQESVTEQDSKDSTYSLSSTLTLSKADYEKHKL
YACEVTHQGLSSPVTKSFNRGEC
SEQ ID NO. 32
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 41)
and heavy (SEQ ID NO. 42) chains of TPP-2084;
CDRs of both the heavy and light chain are
underlined.
TPP-2084
SEQ ID NO. 41
DIQMTQSPSSLSASVGDRVTITCRASQSISSYLNWYQQKPGKAPKLLI
YAASSLQSGVPSRFSGSGSGTDFTLTISSLQPEDFATYYCQQSYSTPG
ITFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC
SEQ ID NO. 42
EVQLLESGGGLVQPGGSLRLSCAASGFTFSPYPMMWVRQAPGKGLEWV
SYISPSGGSTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 51)
and heavy (SEQ ID NO. 52) chains of TPP-2077;
CDRs of both the heavy and light chain are
underlined.
TPP-2077
SEQ ID NO. 51
DIQMTQSPATLSASVGDRVTITCRASQSISSYLNWYQQKPGKAPKLLI
YAASSLQSGVPSRFSGSGSGTDFTLTISSLQPEDFATYYCQQSYSSPG
ITFGPGTKVEIKRTVAAPSVFIFPPSDEQLKSGTASVVCLLNNFYPRE
AKVQWKVDNALQSGNSQESVTEQDSKDSTYSLSSTLTLSKADYEKHKL
YACEVTHQGLSSPVTKSFNRGEC
SEQ ID NO. 52
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 61)
and heavy (SEQ ID NO. 62) chains of TPP-1538;
CDRs of both the heavy and light chain are
underlined.
TPP-1538
SEQ ID NO. 61
AQDIQMTQSPATLSASVGDRVTITCRASQSISSYLNWYQQKPGKAPKL
LIYAASSLQSGVPSRFSGSGSGTDFTLTISSLQPEDFATYYCQQSYSS
PGITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 62
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 81)
and heavy (SEQ ID NO. 82) chains of TPP-1854;
CDRs of both the heavy and light chain are
underlined.
TPP-1854
SEQ ID NO. 81
AQDIQMTQSPATLSASVGDRVTITCRASQSISGYLNWYQQKPGKAPKL
LIYNASSLQSGVPSRFSGSGSGTDFTLTISSLQPEDFATYYCQQSYTS
PFITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 82
EVQLLESGGGLVQPGGSLRLSCAASGFTFSPYPMIWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light (SEQ ID NO. 91)
and heavy (SEQ ID NO. 92) chains of TPP-1853;
CDRs of both the heavy and light chain are
underlined.
TPP-1853
SEQ ID NO. 91
AQDIQMTQSPATLSASVGDRVTITCRASQSISSYLNWYQQKPGKAPKL
LIYNASSLQSGVPSRFSGSGSGTDFTLTISSLQPEDFATYYCQQSYTS
PGITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 92
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light
(SEQ ID NO. 101) and heavy (SEQ ID NO. 102)
chains of TPP-1857; CDRs of both the heavy
and light chain are underlined.
TPP-1857
SEQ ID NO. 101
AQDIQMTQSPATLSASVGDRVTITCRASQSISGYLNWYQQKPGKAPKL
LIYNASSLQSGVPSRFSGSGSGTDFTLTISSLQPEDFATYYCQQSYTS
PGITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 102
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG
Amino acid sequences of the light
(SEQ ID NO. 111) and heavy (SEQ ID NO. 112)
chains of TPP-1858; CDRs of both the heavy
and light chain are underlined.
TPP-1858
SEQ ID NO. 111
AQDIQMTQSPATLSASVGDRVTITCRASQSISSYLNWYQQKPGKAPKL
LIYNASSLQSGVPSRFSGSGSGTDFTLTISSLQPEDFATYYCQQSYTS
PFITFGPGTKVEIKRTVAAPSVFIFPPSDEQLKSGTASVVCLLNNFYP
REAKVQWKVDNALQSGNSQESVTEQDSKDSTYSLSSTLTLSKADYEKH
KLYACEVTHQGLSSPVTKSFNRGEC
SEQ ID NO. 112
EVQLLESGGGLVQPGGSLRLSCAASGFTFSPYPMMWVRQAPGKGLEWV
SYISPSGGKTHYADSVKGRFTISRDNSKNTLYLQMNSLRAEDTAVYYC
ARGGDTYFDYFDYWGQGTLVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIE
KTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMH
EALHNHYTQKSLSLSPG

### EXAMPLE 2

### Biochemical Characteristics of the Antibody

### Determination of Binding Affinities by Biacore Analysis:

Binding affinities of anti-TWEAKR antibodies were determined by surface plasmon resonance analysis on a Biacore T100 instrument (GE Healthcare Biacore, Inc.). Antibodies were immobilized onto a CM5 sensor chip through an indirect capturing reagent, anti-human IgG(Fc). Reagents from the “Human Antibody Capture Kit” (BR-1008-39, GE Healthcare Biacore, Inc.) were used as described by the manufacturer. Anti-TWEAKR antibodies were injected at a concentration of 10 μg/ml at 10 μl/min for 10 sec.

TABLE 3
List of recombinant antigen (TWEAKR) used for affinity measurement

| | | | Cat. No. | |
|---|---|---|---|---|
| | | | (Fitzgerald | SEQ |
| Nomenclature | Description | Origin | Inc) | ID NO |
| TPP-2305 | hTNFRSF12Aaa28-80 | Human | 30R-AT080 | 168 |

TABLE 4
List of antibodies used for affinity measurements

SEQ ID NO

| Nomenclature | Description | Light chain | Heavy chain |
|---|---|---|---|
| P3G5 (TPP- | Murine IgG2a | 121 | 122 |
| 2195) | | | |
| P4A8 (TPP- | Human IgG1 | 125 | 126 |
| 1324) | | | |
| P2D3 (TPP- | Murine IgG2a | 131 | 132 |
| 2196) | | | |
| 136.1 (TPP- | Murine IgG2a | 123 | 124 |
| 2194) | | | |
| PDL-192 (TPP- | Human IgG1 | 127 | 128 |
| 1104) | | | |
| 18.3.3 (TPP- | Murine IgG2a | 129 | 130 |
| 2193) | | | |
| TPP-883 | Human IgG1 | 71 | 72 |
| TPP-1538 | Human IgG1 | 61 | 62 |
| TPP-2077 | Human IgG1 | 51 | 52 |
| TPP-2084 | Human IgG1 | 41 | 42 |
| TPP-2148 | Human IgG1 | 31 | 32 |
| TPP-2093 | Human IgG1 | 21 | 22 |
| TPP-2149 | Human IgG1 | 11 | 12 |
| TPP-2090 | Human IgG1 | 1 | 2 |

TABLE 5
List of commercially available antibodies used for affinity measurements

| Nomenclature | Description | Cat. No. (Abcam) |
|---|---|---|
| ITEM-1 | Murine IgG1 | ab21359 |
| ITEM-4 | Murine IgG1 | ab21127 |

Various concentrations (200 nM, 100 nM, 50 nM, 25 nM, 12.5 nM, 6.25 nM, 3.12 nM, 1.56 nM) of purified recombinant human TWEAKR protein (TPP-2305, SEQ ID NO:168) were injected in HEPES-EP buffer (GE Healthcare Biacore, Inc.) over immobilized anti-TWEAKR antibodies at a flow rate of 60 μl/min for 3 minutes and the dissociation was allowed for 5 minutes. Sensorgrams were generated after in-line reference cell correction followed by buffer sample subtraction. The dissociation equilibrium constant (K_{D}) was calculated based on the ratio of association (kₒₙ) and dissociation rated (k_{off}) constants, obtained by fitting sensorgrams with a first order 1:1 binding model.

TABLE 6
Monovalent K_{D} values of anti-TWEAKR antibodies measured by Biacore
with TWEAKR protein (TPP-2305 (SEQ ID NO: 168)).

| | ka (1/Ms) | kd (1/s) | K_{D} (nM) |
|---|---|---|---|
| TPP-883 | 4.40E+06 | 9.10E−01 | 205.9 |
| TPP-1538 | 4.20E+06 | 1.10E−01 | 27.6 |
| TPP-2077 | 3.00E+06 | 8.60E−02 | 28.9 |
| TPP-2084 | 4.20E+06 | 1.10E−01 | 27.6 |
| TPP-2148 | 5.10E+06 | 1.30E−01 | 24.5 |
| TPP-2093 | 4.10E+06 | 9.00E−02 | 22.1 |
| TPP-2149 | 8.40E+06 | 1.00E−01 | 12.1 |
| TPP 2090 | 9.10E+06 | 1.10E−01 | 12.4 |
| PDL-192 (TPP- | 1.00E+07 | 3.80E−02 | 3.7 |
| 1104) | | | |
| 136.1 (TPP- | 3.84E+07 | 3.24E−02 | 0.8 |
| 2194) | | | |
| 18.3.3 (TPP- | 1.64E+07 | 2.85E−02 | 1.7 |
| 2193) | | | |
| P4A8 (TPP- | 1.20E+06 | 2.70E−03 | 2.3 |
| 1324) | | | |
| P3G5 (TPP- | 2.31E+06 | 1.22E−03 | 0.5 |
| 2195) | | | |
| P2D3 (TPP- | 1.32E+06 | 5.64E−04 | 0.4 |
| 2196) | | | |
| ITEM-1 | 3.80E+06 | 1.10E−02 | 2.9 |
| ITEM-4 | 2.80E+06 | 2.00E−03 | 0.7 |

The antibodies of the invention were determined to bind TWEAKR with moderate affinity (K_{D} 10-200 nM) whereas some antibodies used for comparison (e.g. PDL-192(TPP-1104), 136.1(TPP-2194), 18.3.3(TPP-2193), P4A8(TPP-1324), P3G5(TPP-2195), P2D3(TPP-2196), ITEM-1, ITEM-4) show high affinity binding (0.7-3.7 nM). Sequences of variable domains of antibodies PDL-192, 136.1, 18.3.3, P4A8, P3G5 and P2D3 were obtained from patents WO2009/020933 and WO2009/140177 and sequences encoding the constant region of human IgG1 and murine IgG2 were added, resulting in full length IgGs PDL-192(TPP-1104), 136.1(TPP-2194), 18.3.3(TPP-2193), P4A8(TPP-1324), P3G5(TPP-2195), P2D3(TPP-2196). The range of affinities measured in this study for other previously described antibodies is well in line with published data: For PDL-192, 18.3.3 and 136.1, K_{D} values of 5.5, 0.2 and 0.7 nM were published (WO2009/020933); for P4A8 2.6 nM (WO2009/140177). For comparison, the native ligand TWEAK binds TWEAKR with a K_{D} value of 0.8-2.4 nM (Immunity. 2001 Nov.; 15(5):837-46; Biochem J. 2006 Jul. 15; 397(2):297-304; Arterioscler Thromb Vasc Biol. 2003 Apr. 1; 23(4):594-600).

As a result the antibodies of the invention (TPP-883, TPP-1538, TPP-2077, TPP-2084, TPP-2148, TPP-2093, TPP-2149 and TPP-2090) bind TWEAKR with moderate affinity (K_{D} 10-200 nM).

### Analysis of Species Cross Reactivity by Biacore Analysis:

For the analysis of species cross reactivity, human, rat, murine, dog, pig and macaca fascicularis TWEAKR were expressed and purified as human Fc fragment fusion proteins and immobilized onto a CM5 sensor chip using amine coupling via a standard EDC/NHS-mediated chemistry (BR-1006-33, GE Healthcare Biacore, Inc.).

TABLE 7
List of recombinant proteins used in ELISA for profiling interspecies
binders

| Nomenclature | Description | SEQ ID NO |
|---|---|---|
| TPP-1846 | MAC-TNFRSF12Aaa28-80-hIgG1-Fc | 133 |
| TPP-1779 | RAT-TNFRSF12Aaa28-80-hIgG1-Fc | 134 |
| TPP-1778 | PIG-TNFRSF12Aaa28-80-hIgG1-Fc | 135 |
| TPP-1777 | DOG-TNFRSF12Aaa28-80-hIgG1-Fc | 136 |
| TPP-599 | HUMAN-TNFRSF12Aaa28-80-hIgG1-Fc | 138 |
| TPP-601 | MURINE-TNFRSF12Aaa28-80-hIgG1-Fc | 137 |

Various concentrations (200 nM, 100 nM, 50 nM, 25 nM, 12.5 nM, 6.25 nM, 3.12 nM, 1.56 nM) of anti-TWEAKR antibodies were injected in HEPES-EP buffer (GE Healthcare Biacore, Inc.) over the immobilized TWEAKR species at a flow rate of 60 μl/min for 3 minutes and the dissociation was allowed for 5 minutes. Sensorgrams were generated after in-line reference cell correction followed by buffer sample subtraction. The dissociation equilibrium constant (K_{D}) was calculated based on the ratio of association (kₒₙ₁) and dissociation rated (k_{off1}) constants, obtained by fitting sensorgrams with a bivalent analyte model using Biavaluation Software (version 4.0). The species cross reactivity of anti-TWEAKR antibodies has been determined in “avidity mode” with immobilized bivalent antigen which does not provide “absolute” K_{D} values, but gives good comparative data.

TABLE 8
K_{D} values (nM) of anti-TWEAKR antibodies to different
species measured by Biacore

| | mac fasc | dog | pig | rat | murine | human |
|---|---|---|---|---|---|---|
| | TWEAKR | TWEAKR | TWEAKR | TWEAKR | TWEAKR | TWEAKR |
| | Fc | Fc | Fc | Fc | Fc | Fc |
| TPP-1538 | 21.1 | 25.0 | 22.7 | 20.4 | 4.7 | 15.4 |
| TPP-2077 | 16.8 | 24.5 | 22.4 | 21.2 | 4.2 | 10.3 |
| TPP-2084 | 23.8 | 17.0 | 34.2 | 21.1 | 4.0 | 16.2 |
| TPP-2090 | 21.2 | 6.0 | 9.9 | 4.3 | 4.1 | 15.0 |

As a result the antibodies of the invention (TPP-1538, TPP-2077, TPP-2084 and TPP-2090) show affinity to all tested species (human, rat, murine, dog, pig and macaca fascicularis TWEAKR).

### Characterization of the Binding Epitope of TPP-2090 by N- and C-Terminal Truncation Variants of the TWEAKR Ectodomain:

The alignment of the TWEAKR cysteine rich domain (aa 34-68) of different species (FIG. 1) shows that it is well conserved throughout all 6 analyzed species. PDL-192 binds dependent of R56 (WO2009/020933: FIG. 2B) and therefore does not bind to rat, pig and mouse TWEAKR. TPP-2090 binds dependent of the conserved amino acid D47 and therefore binds to all depicted species.

In a first approach to characterize the binding epitope of the aforementioned antibodies, a N- and C-terminal truncation mutant of the TWEAKR ectodomain was generated and tested for its ability to bind to the different anti-TWEAKR antibodies. N-terminally, amino acids 28 to 33 and C-terminally amino acids 69 to 80 were deleted, thus the cysteine rich domain with disulfide bridges between Cys36-Cys49, Cys52-Cys67 and Cys55-Cys64 remains intact (compare FIG. 2). Both constructs, the full ectodomain 28-80 including the N- and C-terminus and the truncated ectodomain 34-68 were expressed and purified as Fc fusion proteins TPP-2202 and TPP-2203, respectively.

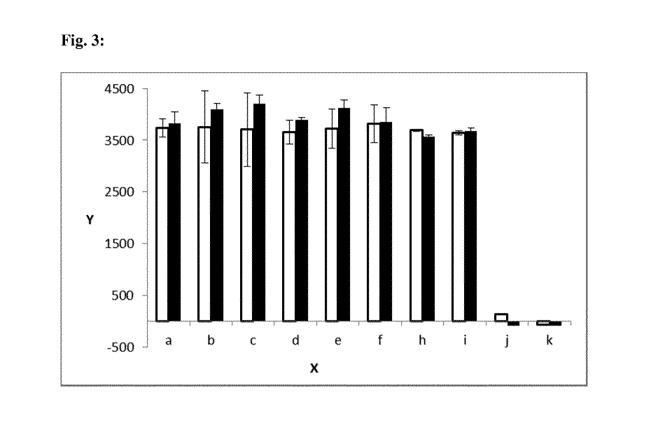

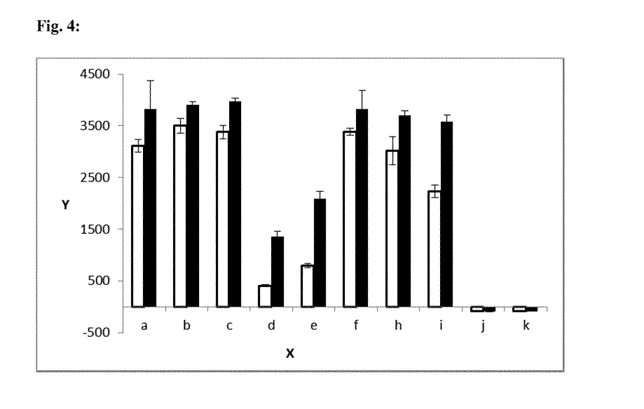

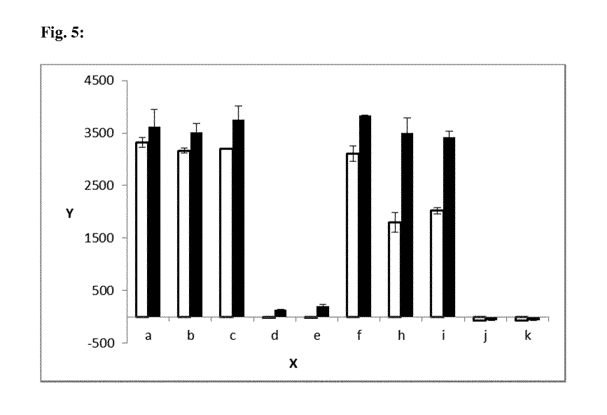

To analyze for binding, 1 μg/ml of the respective dimeric TWEAKR-Fc construct were coated and, 0.3 μg/ml and 0.08 μg/ml of biotinylated IgG were used as soluble binding partner. Detection was done with Streptavidin-HRP and Amplex-Red substrate. IgGs were biotinylated using an approximately 2-fold molar excess of biotin-LC-NHS (Pierce; Cat. No. 21347) according to manufacturer's instructions and desalted using Zeba desalting columns (Pierce; Cat. No. 89889). At all applied concentrations of the soluble ligand, the antibodies of the present invention show saturated binding to both constructs, whereas antibodies P4A8(TPP-1324), P3G5(TPP-2195) and ITEM-4 show saturated binding only to the full length ectodomain and impaired binding to the N- and C-terminally truncated construct (FIG. 3 & FIG. 4). This indicates that the binding epitope of the antibodies of the present invention is located within the cysteine rich domain between amino acid 34-68. To analyze whether the N-terminus or the C-terminus of the TWEAKR ectodomain is needed for P4A8(TPP-1324) and P3G5(TPP-2195) binding a monomeric ectodomain with the C-terminal deletion of amino acids 69 to 80 was generated. The binding of P4A8(TPP-1324) and P3G5(TPP-2195) to the C-terminally truncated TWEAKR ectodomain is also impaired whereas the antibodies of the present invention show saturated binding (FIG. 5).

TABLE 9
List of recombinant antigens used in ELISA analysis for epitope profiling

| Nomenclature | Description | SEQ ID NO |
|---|---|---|
| TPP-2202 | TWEAKR-ECD-28-80-hIgGFc-His | 139 |
| TPP-2203 | TWEAKR-ECD-34-68-hIgGFc-His | 140 |
| TPP-1984 | hTNFRSF12Aaa28-68-CT-His | 141 |

TABLE 10
List of antibodies used in ELISA analysis for epitope profiling

| | | SEQ ID NO | |
|---|---|---|---|
| Nomenclature | Description | Light chain | Heavy chain |
| P3G5 (TPP- | Murine IgG2a | 121 | 122 |
| 2195) | | | |
| P4A8 (TPP- | Human IgG1 | 125 | 126 |
| 1324) | | | |
| 136.1 (TPP- | Mmurine IgG2a | 123 | 124 |
| 2194) | | | |
| PDL-192 (TPP- | Human IgG1 | 127 | 128 |
| 1104) | | | |
| TPP 2090 | Human IgG1 | 1 | 2 |
| TPP-2084 | Human IgG1 | 41 | 42 |

Thus, the binding epitope of TPP-2090, TPP-2084, PDL-192(TPP-1104) and 136.1(TPP-2194) is located within the cysteine rich domain and the binding epitope of P4A8(TPP-1324) and P3G5(TPP-2195) is located at least partially outside of the cysteine rich domain.

### Effect of TWEAKR-Fc Muteins on Antibody Affinity:

To define the binding characteristics of the antibodies of the invention in more detail certain muteins of TWEAKR that have been proposed to be relevant for binding of known agonistic antibodies were tested (WO2009/140177). Therefore, the full ectodomain (amino acids 28-80) with the following single amino acid substitutions were expressed and purified as Fc fusion proteins: T33Q; S40R; W42A; M50A; R56P; H60K; L65Q.

TABLE 11
List of recombinant proteins used in ELISA analysis for mutein binding

| Nomenclature | Description | SEQ ID NO |
|---|---|---|
| TPP-1990 | hTNFRSF12Aaa28-80-L65Q-hIgG1-Fc | 142 |
| TPP-1989 | hTNFRSF12Aaa28-80-H60K-hIgG1-Fc | 143 |
| TPP-2683 | hTNFRSF12Aaa28-80-R56P-hIgG1-Fc | 144 |
| TPP-1988 | hTNFRSF12Aaa28-80-M50A-hIgG1-Fc | 145 |
| TPP-1985 | hTNFRSF12Aaa28-80-W42A-hIgG1-Fc | 146 |
| TPP-1987 | hTNFRSF12Aaa28-80-S40R-hIgG1-Fc | 147 |
| TPP-1986 | hTNFRSF12Aaa28-80-T33Q-hIgG1-Fc | 148 |
| TPP-599 | hTNFRSF12Aaa28-80-hIgG1-Fc | 138 |

To collect dose-response data, the different TWEAKR-Fc muteins were coated with a low concentration (62 ng/ml) in a 384-well Maxisorb ELISA plate and a serial 2fold dilution of biotinylated IgG starting with a concentration of 100 nM was used as soluble binding partner. Detection was done with Streptavidin-HRP and Amplex Red. The tested IgGs were TPP-2090 and TPP-2084 of the current invention, PDL-192, 136.1 and 18.3.3 from WO2009/020933, P4A8 and P3G5 from WO2009/140177, and ITEM-1 and ITEM-4 from Nakayama et al [Biochem Biophys Res Com 306: 819-825].

TABLE 12
List of antibodies used in ELISA analysis for mutein binding

| | | SEQ ID NO | |
|---|---|---|---|
| Nomenclature | Description | Light chain | Heavy chain |
| P3G5 (TPP-2195) | Murine IgG2a | 121 | 122 |
| P4A8 (TPP-1324) | Human IgG1 | 125 | 126 |
| 136.1 (TPP-2194) | Murine IgG2a | 123 | 124 |
| PDL-192 (TPP-1104) | Human IgG1 | 127 | 128 |
| 18.3.3 (TPP-2193) | Murine IgG2a | 129 | 130 |
| TPP 2090 | Human IgG1 | 1 | 2 |
| TPP-2084 | Human IgG1 | 41 | 42 |

TABLE 13
List of commercially available antibodies used in ELISA for mutein
binding

| Nomenclature | Description | Cat. No. (Abcam) |
|---|---|---|
| ITEM-1 | Murine IgG1 | ab21359 |
| ITEM-4 | Murine IgG1 | ab21127 |

IgGs were biotinylated using an approximately 2-fold molar excess of biotin-LC-NHS (Pierce; Cat. No. 21347) according to manufacturer's instructions and desalted using Zeba desalting columns (Pierce; Cat. No. 89889). The dose-response data were fitted and IC50s determined. To visualize the results a table was generated, “−” indicates IC50s above 50 nM, “+” indicates IC50s in the range of 1 to 150 pM.

TABLE 14
Effect of muteins on antibody binding

| | T33Q | S40R | W42A | M50A | R56P | H60K | L65Q | WT |
|---|---|---|---|---|---|---|---|---|
| TPP-2084 | + | + | − | + | + | + | + | + |
| TPP-2090 | + | + | − | + | + | + | + | + |
| PDL- | + | + | − | + | − | + | + | + |
| 192(TPP- | | | | | | | | |
| 1104) | | | | | | | | |
| 136.1(TPP- | + | + | − | + | − | + | + | + |
| 2194) | | | | | | | | |
| 18.3.3(TPP- | + | + | − | + | − | + | + | + |
| 2193) | | | | | | | | |
| P4A8(TPP- | + | + | − | + | + | + | + | + |
| 1324) | | | | | | | | |
| P3G5(TPP- | + | + | − | + | + | + | + | + |
| 2195) | | | | | | | | |
| ITEM1 | + | + | − | + | − | + | + | + |
| ITEM4 | + | + | − | + | + | − | + | + |

As published before, ITEM-4 shows impaired binding to the H60K mutein [WO2009/140177: FIG. 23F] and PDL-192 to the R56P mutein [WO2009/020933: FIG. 22B]. In contrast to published data, ITEM-1 shows impaired binding to R56P and all antibodies to W42A [WO2009/140177: FIG. 23E, FIG. 23F]. This difference can be explained by the method chosen; the extreme low coating concentration favors the discrimination of off-rate impairments since it minimizes avidity effects. As none of the analyzed antibodies shows unimpaired binding to the W42A mutein, this substitution seems to cause rather structural changes and not a direct alteration of the binding epitope.

In contrast to ITEM-1, ITEM-4, PDL-192, 136.1 and 18.3.3, the antibodies of the present invention bind independent of all but W42A substitutions.

### Alanine Scan of Cysteine Rich Domain:

To delineate the binding site of the antibodies of the invention an alanine scan of the cysteine rich domain (amino acids 34-68) was performed. In FIG. 6 it could be shown that N- and C-terminal truncation variants of the full length ectodomain of TWEAKR do not impair binding of the antibodies of the invention. Therefore the binding epitope is localized within the cysteine rich domain. The following substitutions were introduced in the TWEAKR(34-68)-Fc construct: S37A, R38A, S40A, S41A, W42A, S43A, D45A, D47A, K48A, D51A, S54A, R56A, R58A, P59A, H60A, S61A, D62A, F63A and L65A.

TABLE 15
List of TWEAKR mutein constructs for alanine scan of cysteine rich
domain

| Nomenclature | description | SEQ ID NO |
|---|---|---|
| TPP-2203 | TweakR-ECD-34-68-hIgGFc-His | 140 |
| TPP-2625 | TweakR-ECD-34-68-hIgGFc-His-L65A | 149 |
| TPP-2624 | TweakR-ECD-34-68-hIgGFc-His-F63A | 150 |
| TPP-2623 | TweakR-ECD-34-68-hIgGFc-His-D62A | 151 |
| TPP-2622 | TweakR-ECD-34-68-hIgGFc-His-S61A | 152 |
| TPP-2621 | TweakR-ECD-34-68-hIgGFc-His-H60A | 153 |
| TPP-2620 | TweakR-ECD-34-68-hIgGFc-His-P59A | 154 |
| TPP-2619 | TweakR-ECD-34-68-hIgGFc-His-R58A | 155 |
| TPP-2618 | TweakR-ECD-34-68-hIgGFc-His-R56A | 156 |
| TPP-2617 | TweakR-ECD-34-68-hIgGFc-His-S54A | 157 |
| TPP-2616 | TweakR-ECD-34-68-hIgGFc-His-D51A | 158 |
| TPP-2615 | TweakR-ECD-34-68-hIgGFc-His-K48A | 159 |
| TPP-2614 | TweakR-ECD-34-68-hIgGFc-His-D47A | 160 |
| TPP-2613 | TweakR-ECD-34-68-hIgGFc-His-D45A | 161 |
| TPP-2612 | TweakR-ECD-34-68-hIgGFc-His-S43A | 162 |
| TPP-2611 | TweakR-ECD-34-68-hIgGFc-His-W42A | 163 |
| TPP-2610 | TweakR-ECD-34-68-hIgGFc-His-S41A | 164 |
| TPP-2609 | TweakR-ECD-34-68-hIgGFc-His-S40A | 165 |
| TPP-2608 | TweakR-ECD-34-68-hIgGFc-His-R38A | 166 |
| TPP-2607 | TweakR-ECD-34-68-hIgGFc-His-S37A | 167 |

These TWEAKR(34-68)-Fc muteins were expressed in HEK293 cells. To collect dose-response data, IgGs were coated at a concentration of 1 μg/ml in a 384-well Maxisorp ELISA plate and a serial 2fold dilution of the TWEAKR mutein containing supernatant was used as soluble binding partner. Detection was done with anti-HIS-HRP and Amplex Red. The tested IgGs were TPP-2090 of the present invention, PDL-192 from WO2009/020933 and P4A8 from WO2009/140177.

TABLE 16
List of antibodies used for alanine scan of cysteine rich domain

SEQ ID NO

| Nomenclature | Description | Light chain | Heavy chain |
|---|---|---|---|
| P4A8 (TPP- | Human IgG1 | 125 | 126 |
| 1324) | | | |
| PDL-192 (TPP- | Human IgG1 | 127 | 128 |
| 1104) | | | |
| TPP 2090 | Human IgG1 | 1 | 2 |

To assess the relevance of each TWEAKR mutein for binding to different IgGs a correlation blot at a certain mutein concentration was prepared. Exemplarily, in FIG. 6 the correlation blot for the 8fold diluted supernatant of the TWEAKR expression broth is shown with PDL-192(TPP-1104) on the X axis and TPP-2090 on the Y axis. The blot shows that binding of TPP-2090 was impaired by the substitution D47A and binding of PDL-192(TPP-1104) by substitution R56A. For all constructs no binding to P4A8(TPP-1324) could be detected which is in line with the results obtained before (FIG. 6). Thus, the P4A8 epitope is at least partially localized outside of the cysteine rich domain. The identified dependencies on certain TWEAKR amino acids for antibody interaction correlate with the agonistic activity that has been determined for these antibodies. The native ligand TWEAK shows efficient activation of TWEAKR and binds dependent of Leucin 46 in the cysteine rich domain of TWEAKR (Pellegrini et al, FEBS 280:1818-1829). P4A8 shows very low agonistic activity and at least partially interacts with domains outside of the cysteine rich domain of TWEAKR. PDL-192 shows moderate agonistic activity and binds dependent of R56 to the cysteine rich domain but opposite to the TWEAK ligand site. TPP-2090 and TWEAK bind dependent on D47 and L46, respectively, and therefore bind to a similar binding site (FIG. 7).

To support the evidence of a common epitope for all antibodies of this invention further antibodies (namely TPP-2090, TPP-2149, TPP-2093, TPP-2148, TPP-2084, TPP-2077, TPP-1538, TPP-883, TPP-1854, TPP-1853, TPP-1857, TPP-1858) were tested. All antibodies, which have been tested, specifically bind to the D at position 47 (D47) of TWEAKR (see FIG. 6C). Again PDL-192(TPP-1104) is still capable of binding to D47A mutein of TWEAKR.

In conclusion, the antibodies of the invention (e.g. TPP-2090) bind to TWEAKR dependent on D47.

The identified dependencies on certain TWEAKR amino acids for antibody interaction correlate with the agonistic activity that has been determined for these antibodies. The native ligand TWEAK shows efficient activation of TWEAKR and binds dependent on Leucin 46 in the cysteine rich domain of TWEAKR (Pellegrini et al, FEBS 280:1818-1829). P4A8 shows very low agonistic activity and at least partially interacts with domains outside of the cysteine rich domain of TWEAKR. PDL-192 shows moderate agonistic activity and binds dependent of R56 to the cysteine rich domain but opposite to the TWEAK ligand site. Antibodies of this invention (see FIG. 6C) bind dependent on D47, and TWEAK binds dependent on L46, and binds to a similar but distinguishable binding site (FIG. 7). Therefore the antibodies of this invention which show a strong agonistic activity bind to a novel epitope (D47 dependent) for antibodies which is connected to very strong agonistic activity. Interestingly, Michaelson et al (see page 369, left column in Michaelson J S et al, MAbs. 2011 Jul.-Aug.; 3(4):362-75) gave an explanation why all agonistic antibodies examined by them have weaker agonistic activity compared to the natural ligand TWEAK. In their conclusion, the decreased efficacy might be a function of the dimeric binding interaction of an antibody with TWEAKR wherein TWEAK presumably engages in a trimeric interaction. Therefore, it is a surprising finding that an antibody of the invention, though in a dimeric interaction with TWEAKR has even higher agonistic activity. This surprising effect is coupled to the specific binding property of the antibodies of the invention, hence specific binding to D47 of TWEAKR.

### Characterization of Antibodies of the Invention by Epitope Competition Experiments:

To understand the difference of antibodies of this invention and other known anti-TWAEKR antibodies competition experiments were performed. This investigation of overlapping binding motifs for several anti-TWEAKR antibodies has been performed by surface plasmon resonance analysis on a Biacore T100 instrument (GE Healthcare Biacore, Inc.).

TABLE 17
List of antibodies used for competition experiments

SEQ ID NO

| Nomenclature | Description | Light chain | Heavy chain |
|---|---|---|---|
| P3G5 (TPP- | Murine IgG2a | 121 | 122 |
| 2195) | | | |
| P4A8 (TPP- | Human IgG1 | 126 | 126 |
| 1324) | | | |
| 136.1 (TPP- | Murine IgG2a | 123 | 124 |
| 2194) | | | |
| PDL-192 (TPP- | Human IgG1 | 127 | 128 |
| 1104) | | | |
| 18.3.3 (TPP- | Murine IgG2a | 129 | 130 |
| 2193) | | | |
| TPP-2084 | Human IgG1 | 41 | 42 |
| TPP-2090 | Human IgG1 | 1 | 2 |

TABLE 18
List of commercially available antibodies used for competition
experiments

| Nomenclature | Description | Cat. No. (Abcam) |
|---|---|---|
| ITEM-1 | Murine IgG1 | ab21359 |
| ITEM-4 | Murine IgG1 | ab21127 |

TABLE 19
List of recombinant antigen used for competition experiments

| | | | Cat. No. | |
|---|---|---|---|---|
| | | | (Fitzgerald | SEQ |
| Nomenclature | Description | Origin | Inc) | ID NO |
| TPP-2305 | hTNFRSF12Aaa28-80s | Human | 30R- | 168 |
| | | | AT080 | |

All antibodies were immobilized directly onto a CM5 sensor chip using the “Amine coupling Kit” (BR-1006-33, GE Healthcare Biacore, Inc.). Reagents have been used as described by the manufacturer. For saturation of the 1st antibody (immobilized antibody) with antigen, 200 nM TWEAKR (TPP-2305) in HEPES-EP buffer (GE Healthcare Biacore, Inc.) was injected at 30 μl/min for 120 sec. Subsequently 200 nM of the 2nd antibody (“competing antibody”) in HEPES-EP buffer were injected into the flow cell at 30 μl/min for 120 sec. Generally, sensorgrams were generated after in-line reference cell correction followed by sample buffer subtraction. The qualitative competition data (FIG. 8) has been generated by thorough manual inspection of the sensorgrams using Biavaluation Software (version 4.0). A lack of a second binding event after injection of the 2nd antibody indicated clear competition within a respective antibody pair. Non competing antibody pairs showed clear binding signal over background after 2nd antibody injection. In addition, self-competition (1st & 2nd antibody identical) was monitored as an internal system control. Overall, a matrix of nine versus nine antibodies was included into this analysis.

In general anti-TWEAKR antibodies could be clustered into three distinct “competition groups” (FIG. 9). One group contains exclusively TPP-2084 and TPP-2090, both showing competition to all other tested members. These other members could be split into two separate sets of antibodies, which do not show any competition between each other. Therefore “full” competition with all tested anti-TWEAKR antibodies is unique for TPP-2084 and TPP-2090.

This supports the findings described above that both tested antibodies of the invention bind to a new and unique epitope.

### Selectivity Assessment of the Antibodies of the Invention:

The antibody TPP-2090 of the invention was also tested for binding to other members of the TNF receptor superfamily to assess its selectivity. The TNF receptor superfamily shows very high sequence divergence as depicted in FIG. 10. Most similar to TWEAKR are TNFRSF13C and TNFRSF17 with only about 30% sequence identity. The epitope region itself (cysteine rich domain) has no match in any of the other TNFRSF members (BLAST E-Value=0.7 for best hit). The ectodomains of all 29 known TNF receptor superfamily members were purchased as Fc fusion proteins (Table 20) and 1 μg/ml were coated in a Maxisorp ELISA plate.

To collect dose-response data a serial 3fold dilution of biotinylated IgG starting with a concentration of 2 μM was used as soluble binding partner. Detection was done with anti-hIgG1-HRP and Amplex Red. The tested IgG was TPP-2090 of the current invention. As depicted in FIG. 11 TPP-2090 binds already at a very low concentration of 300 pM in saturation to TWEAKR whereas also at a very high concentration of 75 nM it does not bind to all other 28 TNF receptor superfamily members.

Thus, TPP-2090 binds selectively to TWEAKR.

TABLE 20
List of recombinant proteins used in ELISA for selectivity profiling

| | | | Cat. No. |
|---|---|---|---|
| Protein | Nomenclature | Origin | (R&D Systems) |
| TWEAKR (TNFRSF12) | 1 | Human | 1610-TW-050 |
| Apo-3 (TNFRSF25) | 3 | Human | 943-D3-050 |
| Trail-R1 (TNFRSF10A) | 4 | Human | 347-DR-100/CF |
| Trail-R2 (TNFRSF10B) | 5 | Human | 631-T2-100/CF |
| CD-385 (TNFRSF21) | 6 | Human | 144-DR-100 |
| CD95 (TNFRSF6) | 7 | Human | 326-FS-050/CF |
| Rank (TNFSF11) | 8 | Human | 390-TN-010/CF |
| TNF-R1 (TNFRSF1A) | 9 | Human | 636-R1-025/CF |
| TNF-R2 (TNFRSF1B) | 10 | Human | 1089-R2-025/CF |
| BAFF-R (TNFRSF13C) | 11 | Human | 1162-BR-050 |
| DcR3 (TNFRSF6B) | 12 | Human | 142-DC-100 |
| BCMA (TNFRSF17) | 13 | Human | 193-BC-050 |
| TACI (TNFRSF13B) | 14 | Human | 174-TC-050 |
| OX40 (TNFRSF4) | 15 | Human | 3388-OX-050 |
| CD30 (TNFRSF8) | 16 | Human | 6126-CD-100 |
| CD27 (TNFRSF7) | 17 | Human | 382-CD-100 |
| CD40 (TNFRSF5) | 18 | Human | 1493-CD-050 |
| Osteoprotegerin | 19 | Human | 805-OS-100/CF |
| (TNFRSF11B) | | | |
| EDAR | 20 | Human | 157-ER-100 |
| GITR (TNFRSF18) | 21 | Human | 689-GR-100 |
| HVEM (TNFRSF14) | 22 | Human | 356-HV-100/CF |
| NGF R (TNFRSF16) | 23 | Human | 367-NR-050/CF |
| Trail R3 (TNFRSF10C) | 24 | Human | 630-TR-100/CF |
| Lymphotioxin β R | 25 | Human | 629-LR-100 |
| Trail R 4 (TNFRSF10D) | 26 | Human | 633-TR-100 |
| EDA2R (TNFRSF27) | 27 | Human | 1093-XD-050 |
| TROY (TNFRSF19) | 28 | Human | 1548-TR-100 |
| RELT (TNFRSF19L) | 29 | Human | 1385-RT-050 |
| 4-1BB (TNFRSF9) | 30 | Human | 838-4B-100 |

### EXAMPLE 3

### Binding of Anti-TWEAKR Antibodies to Cell Surface of Cancer Cell Lines

To determine the binding characteristics of the anti-TWEAKR antibodies on mouse and human cancer cell lines, binding was tested by flow cytometry to a panel of cell lines. Adherent cells were washed twice with PBS without Ca and Mg (Biochrom #L1825: aqueous solution containing 8000 mg/l NaCl, 200 mg/l KCl, 1150 mg/l Na₂HPO₄, and 200 mg/l KH₂PO₄) and detached by enzyme-free PBS based cell dissociation buffer (Invitrogen). Cells were suspended at approximately 10⁵ cells/well in FACS buffer (PBS without Ca/Mg, containing 3% FCS, Biochrom). Cells were centrifuged (250 g, 5 min, 4° C.) and supernatant discarded. Cells were resuspended in dilutions of the antibodies of interest (10 μg/ml in 80 μl if not indicated otherwise) in FACS buffer, and incubated on ice for 1 h. In the following cells were washed once with 1000 cold FACS buffer and 80 μl secondary antibody diluted at 1:150 (PE goat anti-human IgG, Dianova #109-115-098, or PE Goat Anti-Mouse IgG, Jackson Immuno Research #115-115-164) was added. After incubation for 1 h on ice cells were again washed with cold FACS buffer, resuspended in 1000 FACS buffer and analyzed by flow cytometry using a FACS-Array (BD Biosciences). Results are calculated as Geo Mean of fluorescence detected by the antibody of interest subtracted by background fluorescence as measured by detection with the secondary antibody alone. Values are scored according to the following system: Geo Mean−Geo Mean of secondary antibody alone >10: +, >100: ++, >1000: +++, 10000: ++++, close to category border in ( ). The sources of the cell lines are given in Table 21.

As shown in Table 21, all anti-TWEAKR antibodies of this invention used at a concentration of 10 μg/ml bind a broad range of tumor cells expressing TWEAKR of murine (4T1, Lewis Lung) and human (all other cell lines included in the table) origin representing a variety of tumor entities.

TABLE 21
Binding of anti-TWEAKR antibodies (10 μg/ml) to different cell lines by
scoring of FACS analysis: TPP-1538 and TPP-2090 bind to a broad panel
of murine and human tumor cell lines representing a variety of tumor indications.

| | | | | TPP- | TPP- |
|---|---|---|---|---|---|
| Tumor Entity | Cell Line | Source | Media* | 1538 | 2090 |
| NSCLC | A549 | DSMZ ACC107 | 1 | ++ | ++ |
| | EKVX | NCI 60-Panel, lot 502463 | 2 | ++ | n.d. |
| | NCI-H322 | ECACC 95111734 | 2 | +(+) | ++ |
| | Calu-6 | ATCC HTB-56 | 2 | ++ | n.d. |
| | NCI-H520 | ATCC HTB-182 | 2 | − | n.d. |
| | NCI-H1975 | ATCC CRL-5908 | 2 | ++(+) | n.d. |
| | NCI-H460 | ATCC HTB-177 | 1 | ++ | ++ |
| SCLC | NCI-H69 | ATCC HTB-119 | 2 | − | n.d. |
| CRC | WiDr | ATCC CCL-218 | 4 | ++(+) | +++ |
| | HT-29 | DSMZ ACC299 | 1 | ++ | ++(+) |
| | Lovo | DSMZ ACC350 | 2 | (+) | n.d. |
| | SW-480 | DSMZ ACC313 | 2 | ++ | n.d. |
| HNSCC | A253 | ATCC HTB-41 | 11 | +(+) | n.d. |
| | HSC-3 | JCRB #JCRB0623 | 4 | +(+) | n.d. |
| | SCC4 | DSMZ ACC618 | 10 | +++ | +++ |
| | Fadu | ATCC HTB-43 | 4 | ++ | ++(+) |
| RCC | 786-O | ATCC CRL-1932 | 1 | +++(+) | ++++ |
| PancCA | BxPC3 | ATCC CRL-1687 | 2 | ++(+) | n.d. |
| | As-PC1 | ATCC CRL-1682 | 2 | +(+) | n.d. |
| | MiaPaca2 | ATCC CRL-1420 | 9 | + | n.d. |
| OvCa | SK-OV-3 | ATCC HTB-77 | 3 | ++(+) | +++ |
| BreastCA | MDA-MB-231 | ATCC HTB-26 | 1 | ++ | n.d. |
| | MDA-MB-453 | DSMZ ACC-65 | 1 | + | n.d. |
| Melanoma | A375 | ATCC CRL-1619 | 3 | ++ | ++ |
| GastricCA | NCI-N87 | ATCC CRL-5822 | 2 | ++ | n.d. |
| Esophageal CA | Kyse-180 | DSMZ ACC379 | 2 | (+) | n.d. |
| Hematological CA | Jurkat | ATCC TIB-152 | 2 | − | n.d. |
| | Kasumi-2 | DSMZ ACC526 | 2 | − | n.d. |
| Bladder CA | Scaber | ATCC HTB-3 | 8 | ++ | ++ |
| HCC | SK-Hep1 | DSMZ ACC141 | 7 | ++(+) | +++ |
| | Huh7 | JCRB JCRB0403 | 6 | n.d. | ++ |
| | HepG2 | ATCC HB-8065 | 3 | n.d. | ++ |
| | Hep3B2.1-7 | ATCC HB-8064 | 4 | n.d. | ++(+) |
| | PLC-PRF5 | ATCC CRL8024 | 2 | n.d. | ++ |
| Prostate CA | PC3 | DSMZ ACC465 | 1 | ++ | ++(+) |
| Neuroblastoma | SKNAS | ATCC CRL-2137 | 5 | n.d. | +(+) |
| Murine CA cell | Lewis Lung | ATCC CRL-1642 | 3 | + | n.d. |
| lines | | | | | |
| | 4T1 | ATCC CRL-2539 | 2 | +(+) | n.d. |

(Geo Mean-Geo Mean of secondary antibody alone >10: +, >100: ++, >1000: +++, >10000: ++++, close to category border in ( ))

*List of growth media for cancer cell lines from Table 21:
1. DMEM/Ham's F12; (Biochrom;# FG 4815, with stable Glutamin), 10% FCS
2. RPMI 1640; (Biochrom;# FG 1215, with stable Glutamin), 10% FCS
3. DMEM; (Biochrom;# FG 0435, with stable Glutamin), 10% FCS
4. MEM Earle's; (Biochrom;# FG 0325, with stable Glutamin), 10% FCS
5. DMEM; (Biochrom;# FG 0435, with stable Glutamin) L-Alanyl-L-Glutamin; (2 mM extra for final 4 mM, Biochrom, # K 0302) Non Essentiell Amino Acids; (final: 1×, Biochrom; # K 0293), 10% FCS
6. DMEM; (Biochrom;# FG 0445, high glucose with stable Glutamin), 10% FCS
7. RPMI 1640; (Biochrom;# FG 1215, with stable Glutamin), 20% FCS
8. MEM Earle's; (Biochrom;# F 0315), L-Alanyl-L-Glutamine; (final: 2 mM, Biochrom; # K 0302) Non Essentiell Amino Acids; (final: lx, Biochrom; # K 0293), 10% FCS
9. DMEM/Ham's F12; (Biochrom;# FG 4815, with stable Glutamin), Horse Serum (final: 2.5%); (Biochrom; # S 9135) 10% FCS
10. DMEM/Ham's F12; (Biochrom;# FG 4815, with stable Glutamin), Hydrocortison; (final: 40 ng/mL, Biochrom; # K 3520), 10% FCS
11. McCoy's 5A; (Biochrom;# F 1015) L-Alanyl-L-Glutamine; (1.5 mM extra, Biochrom; # K 0302), 10% FCS
   Overall, it has to be noted, that the maximal cellular binding of the antibodies of the invention as detected by FACS analysis is moderate compared to other known antibodies. As shown in Table 23 and FIG. 12, the amount of antibody bound to the different cells as detected by FACS analysis is lower as compared to other known antibodies (PDL-192(TPP-1104), P4A8(TPP-1324)) at 10 μg/ml, a concentration where cellular binding of the antibody as detected by FACS analysis has reached its plateau (data not shown).

TABLE 22
List of antibodies used for FACS analysis

SEQ ID NO

| Nomenclature | Description | Light chain | Heavy chain |
|---|---|---|---|
| P4A8(TPP- | Human IgG1 | 125 | 126 |
| 1324) | | | |
| PDL-192(TPP- | Human IgG1 | 127 | 128 |
| 1104) | | | |
| TPP-1538 | Human IgG1 | 61 | 62 |
| TPP-2084 | Human IgG1 | 41 | 42 |
| TPP-2093 | Human IgG1 | 21 | 22 |
| TPP 2090 | Human IgG1 | 1 | 2 |

TABLE 23
Binding of different anti-TWEAKR antibodies 10 μg/ml to a panel of cell lines
by scoring of FACS analysis. GeoMean of Fluorescence measures by detection with a
specific antibody minus GeoMean measured with the secondary antibody only is shown.

| | NCI-H322 | 786-O | HT29 | WiDr | SKOV-3 | A375 |
|---|---|---|---|---|---|---|
| TPP-1538 | 98 | 17467 | 634 | 1186 | 1626 | 229 |
| TPP-2084 | 126 | 16342 | 750 | 1346 | 1664 | 199 |
| TPP-2090 | 291 | 20328 | 1423 | 2424 | 2873 | 561 |
| TPP-2093 | 160 | 18156 | 875 | 1718 | 2181 | 255 |
| P4A8(TPP- | 978 | 30230 | 3666 | 5921 | 5971 | 1582 |
| 1324) | | | | | | |
| PDL-192(TPP- | 330 | 25283 | 2127 | 3563 | 4053 | 865 |
| 1104) | | | | | | |

### EXAMPLE 4

### Induction of Caspase-3/7 Activation in Different TWEAKR Expressing Cell Lines

To determine the level of apoptosis induction, Caspase-3/7 activation was measured after treatment of cancer cells with TWEAK or agonistic anti-TWEAKR antibodies. Therefore HT-29 cells were plated at a density of 4000 cells/75 μl/well in 96 well plates in assay medium (DMEM/Ham's F12, Biochrom #FG4815+10% FCS+100 ng/ml IFN gamma (R&D Systems #285-IF)). 24 h later cells were incubated with antibodies to the TWEAKR (see Table 24), recombinant human TWEAK (R&D, #1090-TW-025/CF, E. coli derived recombinant soluble human TWEAK, Arg93-His249 of accession # Q4ACW9, Entrez Gene ID 8742, with an N-terminal Met and 6-His tag) or corresponding isotype control IgG at various concentrations as indicated. After 24 h incubation with the antibodies, Caspase 3/7 activity was determined by adding 100 μl/well Caspase 3/7 Solution (Promega, #G8093) to the cells, incubation for one hour and reading of luminescence on a VICTOR V (Perkin Elmer).

As shown in FIG. 13 and Table 25, incubation with the antibodies of the invention lead to a stronger maximal induction of Caspase 3/7 as compared to the antibodies described in the art (PDL-192(TPP-1104), P4A8(TPP-1324), 136.1(TPP-2194)) and also as compared to 300 ng/ml recombinant human TWEAK (Table 25). Thus, the antibodies described herein, are superior to the previously described antibodies to induce Caspase 3/7 in HT-29 cells.

To determine whether the strong efficacy of the antibodies of the invention also holds true in other cell lines than HT-29, the capacity to induce Caspase 3/7 by anti-TWEAKR antibodies as compared to recombinant human TWEAK was evaluated in a panel of cell lines. For this analysis the following conditions were used: WiDr cells were plated at 3000 cells/well and incubated for 48 h in the presence of TWEAK or the described antibodies, A253 cells were plated at 2500 cells/well and incubated for 24 h, NCI-H322 cells were plated at 5000 cells/well and incubated for 48 h and 786-O cells were plated at 2500 cells/well and incubated for 48 h. The cells were plated in the media as described in Table 21, for A253, NCI-H322 and 786-O cells 100 ng/ml IFN gamma (R&D Systems #285-IF) was added. 24 h after plating antibodies at 100 μg/ml or TWEAK at 300 ng/ml (100 ng/ml TWEAK for NCI-H322 cells) were added and the cells were further incubated for the time periods indicated above. At the end of the incubation time Caspase 3/7 activity was determined as described for HT-29 cells. The fold induction of Caspase 3/7 was calculated as compared to untreated cells.

As shown in Table 25, all tested antibodies of the invention showed an increased Caspase 3/7 induction in HT-29 cells as compared to other known antibodies and reach a stronger activity as compared to 300 ng/ml TWEAK ligand. This strong efficacy to induce apoptosis in cancer cells (as measured by Caspase 3/7 activation) was also seen in WiDr, A253, NCI-H322 and 786-O cells, where the tested antibodies of the invention induced higher fold-changes as compared to other antibodies and 300 ng/ml TWEAK in most experiments.

TABLE 24
List of antibodies used for Caspase induction assay

SEQ ID NO

| Nomenclature | Description | Light chain | Heavy chain |
|---|---|---|---|
| P4A8(TPP- | Human IgG1 | 125 | 126 |
| 1324) | | | |
| 136.1(TPP- | Murine IgG2a | 123 | 124 |
| 2194) | | | |
| PDL-192(TPP- | Human IgG1 | 127 | 128 |
| 1104) | | | |
| TPP-1538 | Human IgG1 | 61 | 62 |
| TPP-2084 | Human IgG1 | 41 | 42 |
| TPP-2093 | Human IgG1 | 21 | 22 |
| TPP-2090 | Human IgG1 | 1 | 2 |
| TPP-1853 | Human IgG1 | 91 | 92 |
| TPP-1854 | Human IgG1 | 81 | 82 |
| TPP-1857 | Human IgG1 | 101 | 102 |
| TPP-1858 | Human IgG1 | 111 | 112 |

TABLE 25
Fold induction of Caspase 3/7 in different cancer cells after incubation with
100 μg/ml anti-TWEAKR antibodies or recombinant human TWEAK (300 ng/ml or
*100 ng/ml). Results from 1-3 representative experiment carried out in triplicates are
shown, including standard deviations. Tested antibodies of the invention show
enhanced induction of Caspase 3/7 in different cell types as compared to known
antibodies or recombinant TWEAK.

| | HT-29 | WiDr | A253 | NCI-H322 | 786-O |
|---|---|---|---|---|---|
| TWEAK | 2.31 ± 0.29 | 1.22 ± 0.02 | 1.53 ± 0.13 | 1.39* ± 0.01 | 1.45 ± 0.05 |
| TPP-1538 | 2.44 ± 0.12 | 1.53 ± 0.11 | n.d. | 1.49 ± 0.07 | 1.81 ± 0.09 |
| TPP-1853 | 2.53 ± 0.03 | n.d. | n.d. | 1.94 ± 0.10 | 1.88 ± 0.21 |
| TPP-1854 | 2.70 ± 0.14 | n.d. | n.d. | 1.98 ± 0.04 | 2.11 ± 0.19 |
| TPP-1857 | 2.37 ± 0.14 | n.d. | n.d. | 1.89 ± 0.04 | 1.76 ± 0.04 |
| TPP-1858 | 2.41 ± 0.16 | n.d. | n.d. | 2.01 ± 0.12 | 2.22 ± 0.14 |
| TPP-2084 | 2.59 ± 0.14 | n.d. | 1.60 ± 0.10 | 1.58 ± 0.23 | 1.61 ± 0.13 |
| TPP-2090 | 2.84 ± 0.31 | 1.73 ± 0.14 | 1.75 ± 0.18 | 1.95 ± 0.14 | 1.56 ± 0.04 |
| TPP-2093 | 2.54 ± 0.04 | n.d. | n.d. | n.d. | 1.43 ± 0.17 |
| P4A8(TPP- | 1.49 ± 0.24 | 1.12 ± 0.07 | 1.38 ± 0.10 | 1.02 ± 0.01 | 1.10 ± 0.05 |
| 1324) | | | | | |
| PDL- | 1.89 ± 0.17 | 1.15 ± 0.04 | 1.30 ± 0.08 | 1.40 ± 0.3 | 1.16 ± 0.06 |
| 192(TPP- | | | | | |
| 1104) | | | | | |
| 136.1(TPP- | 1.81 ± 0.02 | n.d. | n.d. | n.d. | n.d. |
| 2194) | | | | | |

### EXAMPLE 5

### Inhibition of Proliferation by Agonistic Anti-TWEAKR Antibodies in Cancer Cell Lines

To investigate whether the efficacy of the antibodies of this invention to induce Caspase 3/7 is also reflected by an efficacious inhibition of proliferation of different cancer cell lines, anti-proliferative activity was measured after incubation with the antibodies of the invention as compared to reference antibodies or TWEAK ligand.

Therefore cells were plated in 96well plates in 750 assay medium (growth media from Table 21, plus 100 ng/ml IFN gamma for 786-O cells) at the following cell numbers: WiDr cells 3000 cells/well, 786-O cells 2500 cells/well. 24 h later cells were incubated with anti-TWEAKR antibodies (see Table 26), recombinant human TWEAK or isotype control IgG (not shown) at the indicated concentrations (antibodies from 0.03-300 μg/ml, TWEAK 100 or 300 ng/ml). At the time of antibody addition cell viability was determined in sister plates (time point zero): therefore 750/well CTG solution (Promega Cell Titer Glo solution (catalog # G755B and G756B) was added to the cells, incubated for 10 minutes and luminescence was read on a Victor V (Perkin Elmer). 96 h after incubation with the agents, cell viability was determined in the assay plates by addition of 100 μl/well CTG solution, 10 min incubation and reading of luminescence. Proliferation in control wells was calculated by subtracting time zero values from the luminescence values in the untreated control wells. % of cell proliferation was calculated in the compound treated wells as compared to the untreated control wells.

The resulting dose response curves representing cell proliferation in the treated cells as compared to control cells are shown in FIG. 14A (WiDr cells) and FIG. 14B (786-O cells). All tested antibodies of the current invention inhibited proliferation of WiDr cells by 50-60% and of 786-O cells by 70-80%, which was significantly more than the proliferation inhibition reached by other known antibodies, e.g. PDL-192(TPP-1104) and P4A8(TPP-1324). Thus, the antibodies of the current invention are the most efficacious anti-proliferative anti-TWEAKR antibodies described to date.

To evaluate, whether this strong anti-proliferative activity can also be observed in a broader cell panel, in addition LOVO, NCI-H1975, SW-480 (all 3000 cells/well), HT-29 (4000 cells/well), A253 and SK-OV3 (both 2500 cells/well) cells were incubated with 100 μg/ml anti-TWEAKR antibodies or TWEAK ligand for the time periods as indicated in Table 27. All cells were seeded in the growth media indicated in Table 21 and for all cells except for WiDr cells 100 ng/ml IFNgamma was added to the assay medium when seeding the cells. The percentage of growth inhibition for treated cells as compared to proliferation in untreated control cells was measured and calculated as described above. As shown in Table 27 the antibodies of the current invention are more efficacious as compared to other known antibodies in inhibiting proliferation of various cancer cell lines at 100 μg/ml. In most experiments the antibodies of the current invention also show equal or stronger efficacy as compared to 100-300 ng/ml TWEAK ligand. Thus, the antibodies described in this invention are unique in their activity to induce apoptosis and proliferation inhibition in a broad panel of cancer cell lines.

TABLE 26
List of antibodies used for proliferation assay

SEQ ID NO

| Nomenclature | Description | Light chain | Heavy chain |
|---|---|---|---|
| P4A8(TPP- | hIgG1 | 125 | 126 |
| 1324) | | | |
| PDL-192(TPP- | hIgG1 | 127 | 128 |
| 1104) | | | |
| TPP-1538 | Human IgG1 | 61 | 62 |
| TPP-2084 | Human IgG1 | 41 | 42 |
| TPP-2093 | Human IgG1 | 21 | 22 |
| TPP-2090 | Human IgG1 | 1 | 2 |

TABLE 27
% Inhibition of proliferation induced by incubation with 100 μg/ml anti-
TWEAKR antibodies or TWEAK ligand (*100 ng/ml, **300 ng/ml). Incubation time in
the presence of the agents is indicated as time of assay in [h]. Results from 1-3
representative experiments carried out in triplicates are shown. Antibodies of the
invention show stronger inhibition of cancer cell proliferation as compared to known
antibodies (PDL-192(TPP-1104), P4A8(TPP-1324)) and equal or stronger activity as
compared to recombinant TWEAK.

| | time of | | | | | | | |
|---|---|---|---|---|---|---|---|---|
| | assay [h] | TWEAK | TPP-1538 | TPP-2084 | TPP-2090 | TPP-2093 | PDL-192 | P4A8 |
| 786-O | 96h | 12%* | 75% | 68% | 71% | n.d. | 41% | 29% |
| LOVO | 72h | 53%* | 65% | n.d. | n.d. | n.d. | 49% | −5% |
| NCI-H1975 | 72h | 56%* | 64% | n.d. | n.d. | n.d. | 46% | 20% |
| SW480 | 72h | 0%* | 49% | n.d. | n.d. | n.d. | 27% | 5% |
| WiDr | 96h | n.d. | 55% | 52% | 50% | n.d. | 12% | 4% |
| HT-29 | 24h | 98%** | 97% | 98% | >100% | >100% | 62% | −10% |
| A253 | 72h | >100%** | >100% | >100% | 99% | >100% | 87% | 37% |
| SK-OV3 | 96h | n.d. | >100% | n.d. | n.d. | n.d. | 44% | n.d. |

### EXAMPLE 6

### Cytokine Secretion Induced by Anti-TWEAKR Antibodies from Cancer Cells and Xenograft Tumors

Next, it was of interest to investigate whether the strong agonistic activity of the antibodies of this invention is also seen in the induction of cytokine secretion from cancer cells.

Therefore A375 cells were plated at 2500 cells/well in 96well plates in growth medium DMEM (Biochrom;# FG 0435, with stable Glutamin), 10% FCS. 24 h later cells were incubated with anti-TWEAKR antibodies, recombinant human TWEAK at various concentrations as indicated or corresponding isotype control IgG. 24 h after start of the incubation with the antibodies, cell supernatant or dilutions thereof were added to the Capture Elisa Plate of the human CXCL8/IL-8 ELISA Kit (R&D Systems DY208) and incubated over night at 4° C. by shaking 300 rpm. On the next day, samples were analyzed by using the human CXCL8/IL-8-ELISA Kit (R&D Systems DY208) according to the manufacturer's instructions. Optical density was measured at 450 nm (Tecan Spectra, Rainbow) together with background correction. To calculate absolute levels of IL-8 a standard curve using recombinant human IL-8 protein was applied according to the manufacturer's recommendations (R&D Systems).

As shown in FIG. 15, the antibodies of this invention showed increased induction of IL-8 release as compared to other antibodies previously known. At 100 μg/ml the antibodies of this invention TPP-1538/-1854/-2084/-2090 reached 134/129/113/103% of the activation as compared to 300 ng/ml TWEAK ligand respectively. In contrast, the antibodies used for comparison, PDL-192(TPP-1104)/P4A8(TPP-1324)/136.1(TPP-2194), reached only 66/29/93%, respectively. Thus, the antibodies of this invention show the strongest activity with regard to induction of IL-8 secretion as compared to previously known antibodies and 300 ng/ml TWEAK ligand.

To investigate, whether the observed cytokine secretion is also of relevance in xenograft tumors in mice, human and murine cytokines in serum/plasma from tumor bearing (A375, WiDr) as well as tumor free mice were investigated. 5×10⁶ A375 cells in Matrigel/PBS (1:1) or 5×10⁶ WiDr cells in Matrigel/Medium (1:1) were subcutaneously inoculated in immunodeficient female NMRI nude mice. Parallel to A375-bearing mice, non-tumor mice were investigated. Treatment started 7d after inoculation with established tumors of about 40 mm². Mice were treated by a single intravenous injection of TPP-1538 (10 mg/kg) or TPP-2090 (3 mg/kg) both diluted in PBS into the tail vein. Mice were then sacrificed at given time-points (0, 6 h, 24 h for A375-bearing mice and 0, 7 h, 24 h, 72 h, 168 h, 240 h for WiDr-bearing mice) to harvest serum/plasma samples. Blood was collected after decapitation and serum was prepared by 30 minutes clotting with subsequent centrifugation at 1000×g.

Cytokines were quantified using Luminex® bead immunoassays. Human cytokines were determined with Human Cytokine Magnetic 25-plex panel (Invitrogen®, Cat-No. LHC0009M), comprising IL-1β, IL-1RA, IL-2, IL-2R, IL-4, IL-5, IL-6, IL-7, IL-8, IL-10, IL-12 (p40), IL-13, IL-15, IL-17, TNF-α, IFN-α, IFN-γ, GM-CSF, MIP-1α, MIP-1β, IP-10, MIG, Eotaxin, RANTES, and MCP-1). Murine cytokines were determined with Mouse Cytokine Magnetic 20-plex panel (Invitrogen®, Cat-No. LHC0006M) comprising FGF basic, GM-CSF, IFN-γ, IL-1α, IL-1β, IL-2, IL-4, IL-5, IL-6, IL-10, IL-13, IL-12 (p40/p70), IL-17, IP-10, KC, MCP-1, MIG, MIP-1α, TNF-α and VEGF). The assays were conducted according to the manufacturer's instructions and measured by the Luminex reader Bio-Plex 200 (Bio-Rad GmbH). Cytokine concentrations were interpolated from standard curves, as part of the assay procedure, by the operating software Bio-Plex Manger (Bio-Rad GmbH).

As shown in FIG. 16A, human IL-8 is released from WiDr xenograft by a single treatment with TPP-2090 3 mg/kg in a time dependent manner. In addition, induction of secretion of human MCP-1, IP-10 and IL-15 was observed after treatment with TPP-2090 (not shown).

To further investigate whether the cytokine induction observed in the plasma of tumor bearing mice after treatment with agonistic anti-TWEAKR antibodies of the invention was indeed tumor specific, a similar investigation was carried out in A375 tumor bearing and tumor free mice and human as well as murine cytokines were measured. As shown in FIG. 16B, human IL-8 is released from A375 xenografts in tumor bearing mice 6 h after treatment with TPP-1538 at 10 mg/kg. In addition, increased levels of human MCP-1, IP-10 and IL-1RA were observed (not shown). In contrast, in the plasma of treated tumor free mice no increased secretion of human cytokines was detected (FIG. 16B and not shown). In addition, no increase of murine cytokines including the murine IL-8 analogue KC was detected in the plasma of neither tumor bearing nor tumor free mice after treatment with TPP-1538 (data not shown). To summarize, the antibodies of the present invention potently induce secretion of cytokines from cancer cells and xenografts in vivo in a tumor specific manner.

### EXAMPLE 7

### Internalization of Anti-TWEAKR Antibodies and Usability for Drug Conjugate Approaches

To investigate, whether anti-TWEAKR antibodies of the current invention are potentially usable for the generation of antibody drug conjugates (ADC)s, the internalization capacity of the antibodies was investigated.

To visualize this process the TWEAKR specific antibodies TPP-1538 and TPP-2090 and an isotype control antibody were selected. The antibodies were conjugated in the presence of a twofold molar excess of CypHer 5E mono NHS ester (batch 357392, GE Healthcare) at pH 8.3. After the conjugation the reaction mixture was dialyzed (slide-A-Lyser Dialysis Cassettes MWCD 101(D, Fa. Pierce) overnight at 4° C. to eliminate excess dye and to adjust the pH-value. Afterwards the protein solution was concentrated (VIVASPIN 500, Fa Sartorius Stedim Biotec). In addition to the pH-dependent fluorescent dye CypHer5E the ph-independent dye Alexa 488 was used. The dye load of the antibody was determined with a spectrophotometer (Fa. NanoDrop). The dye load of TPP-1538, TPP-2090 and an isotype control antibody were in a similar range. The affinity of the labeled antibodies was tested in a cell binding-assay to ensure that labeling did not alter the binding to TWEAKR. These labeled antibodies were used in the following internalization assays. Prior to treatment cells (2×10⁴/well) were seeded in 100 μl medium in a 96-MTP (fat, black, clear bottom No 4308776, Fa. Applied Biosystems). After 18 h incubation at 37° C./5% CO2 medium was changed and labeled anti-TWEAKR antibodies TPP-1538 and TPP-2090 were added in various concentrations (10, 5, 2.5, 1, 0.3, 0.1 μg/ml). The identical treatment was carried out with the isotope control antibody (negative control). The incubation time was chosen to be 0, 0.5 h, 1 h, 2 h, 3 h, 6 h and 24 h. The fluorescence measurement was performed with the InCellAnalyzer 1000 (Fa. GE Healthcare). Granule counts per cell and total fluorescence intensity were measured in a kinetic fashion. A highly specific and significant internalization of TPP-1538 and TPP-2090 was observed in endogenous TWEAKR expressing cancer cell lines 786-O (renal cancer) and HT-29 (colon cancer).

This internalization was target dependent as uptake could only be demonstrated using the anti-TWEAKR antibodies while no internalization was observed with the isotype control antibodies. During the first 6 h the anti-TWEAKR antibodies showed a 20-40-fold increase of antibody internalization compared to isotype controls. Isotype control antibodies showed a minor internalization after a long exposure (>24 h). Internalization of anti-TWEAKR antibodies labeled with Alexa 488 upon binding reveals that more than 50% of internalized antibodies seem to follow the endocytotic pathway. In FIG. 17, evaluation of the time course of specific internalization of TPP-1538 and TPP-2090 upon binding to endogenous TWEAKR expressing cells is shown. Internalization of antibodies (1 μg/ml) was investigated on renal cancer cell line 786-O. Granule counts per cell were measured in a kinetic fashion. Rapid internalization could be observed for TPP-1538 and TPP-2090, whereas the isotype control hIgG1 did not internalize. Additionally, a significantly improved internalization efficacy was seen with TPP-2090. The higher efficacy of TPP-2090 could be verified in internalization assays performed using a variety of cancer cells with different receptor levels (not shown).

Additionally, the activity of anti-TWEAKR antibodies to inhibit proliferation of cells when incubated in the presence of saporin-conjugated secondary antibodies was evaluated in Hum-Zap assays. Therefore 786-O cells were plated at 2500 cells/750/well in 96 well plates in growth medium (DMEM/Ham's F12, Biochrom #FG4815+10% FCS). 24 h later 40 nM antibodies (TPP-1538, TPP-2090 or isotype control antibody) were incubated in the presence or absence of 40 nM saporin-conjugated secondary antibodies (Hum Zap, Advanced Targeting Systems Cat #IT-22, Lot 59-83) for 15 min at room temperature. After the incubation time 250 of the reaction mix was added to the cells, resulting in a final concentration of 10 nM antibody in the sample wells. At the time of the antibody addition cell viability was determined in sister plates (time point zero). Therefore, 750/well CTG solution (Promega Cell Titer Glo solution (catalog # G755B and G756B) was added to the cells, incubated for 10 minutes and luminescence was read on a Victor V (Perkin Elmer).

48 h after start of incubation with the antibody/Zap complex, 100 μL/well CTG solution was added to all test wells, incubated for 10 minutes and luminescence was read on a VICTOR V.

As shown in FIG. 18, incubation of 786-O cells with anti-TWEAKR antibodies at 10 nM in the presence of saporin-conjugated secondary antibodies almost completely inhibited cell proliferation, whereas under the same experimental conditions (absence of IFNgamma, 48 h incubation time only) no anti-proliferative activity was observed in the absence of saporin-conjugated secondary antibodies or with isotype control antibodies. To summarize, anti-TWEAKR antibodies of the present invention show rapid internalization and targeted delivery of conjugated payloads and are thus well suitable for the generation and use as ADCs.

### EXAMPLE 8

### Anti-Tumor Efficacy of Anti-TWEAKR Antibodies in Xenograft Models In Vivo

To investigate, whether anti-TWEAKR antibodies show anti-tumor activity in vivo xenograft tumors derived from different cancer cell lines or patient derived tumor models were tested for their sensitivity against tumor growth inhibition by agonistic anti-TWEAKR antibodies in mono- or combination therapy.

Before start of the in vivo experiments expression of TWEAKR in the selected xenograft models was evaluated by immunohistochemistry. Therefore, frozen sections (5 μM) of the corresponding xenografts were fixed with acetone for 5 min at 4° C. and blocked against unspecific protein binding and peroxidase activity. Tissue sections were incubated with rabbit anti-TWEAKR antibody (Fn14, Epitomics, 3488-1) at room temperature for 60 min, followed by peroxidase labeled anti rabbit polymer (DAKO, K4011) incubation for 30 min. Sections were developed with diaminobenzidine and finally counterstained with hematoxylin. Only models that were positive for expression of the TWEAKR were used for in vivo experiments

For the investigation of the anti-tumor activity of the anti-TWEAKR antibodies in vivo, nude mice bearing xenografts from different human tumor cell lines or patient-derived tumors were treated by repeated intravenous injections.

Tumor cell lines were cultivated as described in the parts above and 100 μl containing cell line specific numbers of tumor cells inoculated subcutaneously (s.c.) into female athymic nude mice (NMRI nu/nu, 6-8 weeks, 20-25 g, Taconic). Mice were housed under standardized pathogen free conditions and treated according to the animal welfare guidelines.

After tumor growth to a size of approximately 40 mm² mice were randomized into control and treatment groups with a respective group size of n=8-10. Mice were treated with various doses of anti-TWEAKR antibodies diluted in PBS by intravenous injection (i.v.) into the tail vein with a twice per week schedule (q4dx3: applications twice per week, three applications in total; q4dx8: applications twice per week, eight applications in total). Combination therapy partners such as Regorafenib (10 mg/kg daily, per os) and the PI3K-inhibitor 1 (10 mg/kg, BID, 2d on, 5d off (applications twice daily on two consecutive days, followed by five days without treatment), i.v.) were diluted in their respective formulations whereas the standard of care therapies Irinotecan (5 mg/kg, 4d on, 3d off (applications once daily on four consecutive days followed by three days without treatment) i.v.) and Paclitaxel (16 mg/kg, q7dx4 (applications once per week, four applications in total), i.v.) were diluted in 0.9% NaCl. Animals injected with PBS served as the control (vehicle) group. The applied volume of the compounds was 5 ml/kg body weight per mouse.

Tumor growth was monitored 2-3 times per week by caliper measurement (length×width in mm) as well as body weight (in g). At the end of study tumors were dissected, weighted and used for the calculation of tumor-to-control (T/C) ratios (mean tumor weight of treated animals divided by mean tumor weight of control/vehicle animals). In the human renal cell cancer model 786-O (positive TWEAKR expression) efficacy of the anti-TWEAKR antibodies TPP-2084 and TPP-2090 was tested at three different low doses against the isotype control antibody. 2×10⁶ tumor cells in 100% Matrigel were s.c. inoculated in female nude mice. After 7d established tumors with a size of about 40 mm² were treated with 0.3 mg/kg, 1 mg/kg and 3 mg/kg of antibodies (i.v., q4dx3: applications twice per week, three applications in total). At day 40, comparison of tumor weights after dissection showed dose-dependent efficacy of TPP-2084 and TPP-2090 which was highest at 3 mg/kg (FIG. 19). A clear differentiation against the isotype and vehicle group (treated with PBS) could be demonstrated. No loss of bodyweight was observed in any of the groups. Tumor-to-control ratios listed in Table 28 demonstrate good efficacy in the 786-O model and in further tumor models (A375, A253, SK-OV-3, Bx-PC3, treated with 3-10 mg/kg anti-TWEAKR antibodies TPP-1538, TPP-2094 or TPP-2090 in a q7dx3 (applications once per week, three applications in total) or q4dx3 (applications twice per week, three applications in total) schedule with the exception of MDA-MB-231 where more intense dosing schedules of anti-TWEAKR antibodies might be required to reach monotherapy efficacy.

TABLE 28
Final Tumor-to-Control (T/C) ratios in 786-O and further tumor
models after treatment with TPP-1538, TPP-2084 or TPP-2090.
Anti-TWEAKR antibodies show strong anti-tumor activity in a
variety of xenograft models from different solid tumor
indications.

| | | TWEAKR | | | |
|---|---|---|---|---|---|
| | | Expres- | | | |
| Tumor | | sion | Final T/C, | Final T/C, | Final T/C, |
| Model | Indication | (IHC) | TPP-1538 | TPP-2084 | TPP-2090 |
| 786-O | Renal cell | +++ | 0.57* | 0.34 | 0.45 |
| | cancer | | | | |
| A375 | Melanoma | ++ | 0.46* | | n.d. |
| A253 | Salivary | +++ | 0.43* | 0.65 | 0.64 |
| | gland | | | | |
| | cancer | | | | |
| SK-OV-3 | Ovarian | ++ | 0.55* | n.d. | n.d. |
| | cancer | | | | |
| Bx-PC3 | Pancreatic | +++ | 0.54* | n.d. | n.d. |
| | cancer | | | | |
| MDA-MB- | Breast | +++ | 0.91 | n.d. | n.d. |
| 231 | cancer | | | | |

T/C: tumor-to-control ratio based on final tumor weight after dissection or based on measurement of tumor area (*).

FIG. 20 shows the efficacy of the anti-TWEAKR antibody TPP-2090 in the human colon cancer xenograft WiDr (which represents a subclone of the HT-29 tumor cell line) in monotherapy and combination therapy with Irinotecan and Regorafenib. 5×10⁶ WiDr cells in Matrigel/Medium (1:1) were s.c. inoculated in immunodeficient NMRI nude mice. Treatment started 7d after inoculation with established tumors of about 40 mm². Even 3 mg/kg of TPP-2090 (i.v., q4dx7: applications twice per week, seven applications in total) in monotherapy was strongly effective to control tumor growth. Combination of 3 mg/kg TPP-2090 with either Irinotecan (5 mg/kg, i.v., 4d on, 3d off) or Regorafenib (10 mg/kg, p.o., daily) resulted in additive efficacy with tumor regression. All therapeutic regimens were well tolerated by the mice (max. 4% initial and reversible body weight loss). Final T/C values are listed in Table 29.

TPP-2090 was also investigated in other colorectal tumor models such as SW480 and the patient-derived tumor model Co5682 with similar good results (Table 29). A dose of 10 mg/kg TPP-2090 was effective in monotherapy in SW480 to control tumor growth (T/C 0.49) and to lead to tumor regression in combination with 5 mg/kg Irinotecan (T/C 0.22) or 10 mg/kg Regorafenib (T/C 0.37). In Co5682 xenografts 3 mg/kg of TPP-2090 showed synergistic efficacy with tumor regression in combination with Irinotecan (T/C 0.23) and tumor stasis in combination with Regorafenib (T/C 0.27).

TABLE 29
Final Tumor-to-Control (T/C) ratios of two colon cancer cell lines
WiDr and SW480 and one patient-derived colon cancer xenograft
Co5682 after treatment with TPP-2090 and combination partners
based on tumor weights at study end.

| | T/C (final) | | |
|---|---|---|---|
| Tumor | TPP-2090 | Combo TPP-2090 + | Combo TPP-2090 + |
| Model | mono Tx | Irinotecan | Regorafenib |
| WiDr | 0.17 | 0.04 | 0.07 |
| SW480 | 0.49 | 0.22 | 0.37 |
| Co5682 | 0.97 | 0.23 | 0.27 |

T/C: tumor-to-control ratio based on final tumor weight after dissection,
Tx: therapy,
combo: combination therapy

FIG. 21 shows the efficacy of the anti-TWEAKR antibody TPP-2090 in the human non-small-cell lung cancer xenograft NCI-H322 in monotherapy and combination therapy with Paclitaxel. 5×10⁶ NCI-H322 cells in Matrigel were s.c. inoculated in immunodeficient NMRI nude mice. Treatment started 14d after inoculation with established tumors of about 45 mm². At a dose of 5 mg/kg TPP-2090 (i.v., q4dx8) was strongly effective in monotherapy demonstrating tumor regression. Combination of 10 mg/kg TPP-2090 with Paclitaxel (16 mg/kg, i.v., q7dx4) resulted in slight additive efficacy. All therapeutic regimens were well tolerated by the mice (max. 3% reversible body weight loss). Final T/C values are listed in Table 30.

Again, TPP-2090 was also investigated in other lung cancer models such as NCI-H1975 and the patient-derived tumor models Lu7343 and Lu7433 with comparable results (Table 30). A dose of 3 mg/kg TPP-2090 showed additive effects in NCI-H1975 in combination with 16 mg/kg Paclitaxel resulting in tumor regression (T/C 0.08). Similarly, in the patient-derived NSCLC models Lu7343 and Lu7433 combination of 3 mg/kg TPP-2090 with 10 mg/kg of the PI3K-inhibitor 1 led to tumor control or regression (T/C 0.18-0.36) in an additive efficacious manner.

PI3K-inhibitor 1 is (2-amino-N-[7-methoxy-8-(3-morpholin-4-ylpropoxy)-2,3-dihydroimidazo[1,2-dquinazolin-5-yl]pyrimidine-5-carboxamide dihydrochloride

TABLE 30
Final Tumor-to-Control (T/C) ratios of two NSCLC cell lines
NCI-H322 and -H1975 and two patient-derived lung cancer
xenografts Lu7343 and Lu7433 after treatment with TPP-2090
and combination partners based on tumor weights at study end.

| | T/C (final) | | |
|---|---|---|---|
| | | | Combo TPP- |
| | TPP-2090 | Combo TPP-2090 + | 2090 + PI3K- |
| Tumor Model | mono Tx | Paclitaxel | inhibitor 1 |
| NCI-H322 | 0.17 | 0.14 | — |
| NCI-H1975 | 0.48 | 0.08 | — |
| Lu7343 | 0.65 | — | 0.18 |
| Lu7433 | 0.53 | — | 0.36 |

T/C: tumor-to-control ratio based on final tumor weight after dissection,
Tx: therapy,
combo: combination therapy

All described in vivo examples demonstrate the strong efficacy of the anti-TWEAKR antibody TPP-2090 in a broad panel of cell line-derived and patient-derived human tumor models (all with TWEAKR positive expression) in monotherapy as well as in combination therapy. TPP-2090 was well tolerated by the mice at all doses used.

### EXAMPLE 9

### Mode of Action of Anti-TWEAKR Antibodies in Xenograft Models

To investigate the mode of action of anti-TWEAKR antibodies in vivo, tumors from WiDr xenografts were taken at study end as described in Example 8 and investigated by immunohistochemistry and Western Blot Analysis.

Frozen sections (5 μm) of WiDr xenografts (tumors from 3 individual animals per group) were stained immunohistochemically for the proliferation marker protein Ki67 (see EXAMPLE 8 for details of in vivo experiment). Sections were fixed with freshly prepared 4% paraformaldehyde for 20 min at 4° C. and blocked against unspecific protein binding and peroxidase activity. Murine anti Ki67 antibody (DAKO, M7240) was labeled with biotin according to manufacturer's instruction (DAKO, K3954) and incubated at room temperature for 60 min with the tissue sections, followed by ExtraVidin-peroxidase (DAKO, K3468) incubation for 30 min. Sections were developed with diaminobenzidine and finally counterstained with hematoxylin. For quantification, entire tumor sections were scanned and analyzed using ARIOL automated microscopy version 3.2 (Applied Imaging, San Jose Calif., USA). Representative images of PBS (i.v., q4dx7) and TPP-2090 (10 mg/kg, i.v., q4dx7) treated xenografts stained for Ki67 are shown in FIGS. 22 A and B respectively. The quantification using the ARIOL image system, revealed 355+/−59 Ki67 positive cells/mm² in the group treated with 10 mg/kg TPP-2090 (i.v. q4dx7), and 863+/−90 Ki67 positive cells/mm² in the vehicle treated group. Thus, in line with the observed reduction in tumor volume, treatment with agonistic anti-TWEAKR antibodies leads to a reduction of the proliferation marker Ki67 in xenograft tumors. In addition WiDr xenograft tumors (see Example 8 for details of in vivo experiment), snap frozen at study end were analyzed by Western Blot to evaluate effects of the antibody treatment on Stat-1 and NF-kappaB signaling pathways. Tumors of 4 individual animals per group were cut in slices of around 5 mm diameter and each slice deposited in a 2 ml Eppendorf tube together with a precooled 5 mm steel bull (Qiagen) and 500 μl lysis buffer (50 mM Hepes pH 7.2, 150 mM NaCl, 1 mM MgCl₂, 10 mM Na₄P₂O₇, 100 mM NaF, 10% Glycerin, 1.5% Triton X-100, freshly added Complete Protease Inhibitor cocktail (Roche No. 1873580001), 4 mM Na₃VO₄, pH adjusted to 7.4 with NaOH)). Samples were lysed for 3 min at 300 Hz in a Tissuelyzer (Qiagen) followed by incubation on ice for 30 min. In the following, samples were centrifuged for 10 min at 13000 rpm at 4° C. in a Micro-centrifuge (Eppendorf) and supernatants from one original tumor pooled back together. Protein levels in the tumor lysates were determined by using the BCA protein assay kit (Novagen, lysates 1:50 diluted in H₂0). Samples were diluted to a final concentration of 4 mg/ml and 10 μl of sample were mixed with 3.08 μl of (10*) Sample Reducing agend, 10 μl H₂O and 7.68 μl (4*) NuPAGE Sample Buffer (Invitrogen). Samples corresponding to 40 μg of protein were applied to NuPage 4-12% SDS page gels from Invitrogen and run for 2 h45 min at 120V. Blotting was carried out by an iBlot system (Invitrogen) according to the manufacturer's recommendations. Membranes were blocked for 2 h at room temperature in 5% BLOT QuickBlocker in PBST (Invitrogen), followed by incubation with primary antibodies over night at 4° C. Primary antibodies were as follows: Phospho-Statl #9167S, Stat-1 #9172, both Cell Signaling Technology, dilution 1:1000; TWEAKR/Fn14 #3488-1 Epitomics, dilution 1:10000; NF-kappaB2 p100/p52 #4882S, Cell Signaling Technology, dilution 1:1000 in 3% BLOT QuickBlocker in PBST. On the next day membranes were washed three times in PBST, followed by incubation with secondary antibodies (Peroxidase-conjugated donkey anti-rabbit IgG # NA934, GE Healthcare 1:10000 in 3% BLOT QuickBlocker/PBST) for 2 h at room temperature. Subsequently, membranes were washed four times for 10 min with PBST and signals were detected by chemoluminescence after incubation with ECL reagent. To detect the loading control, membranes were stripped with stripping solution Re-Blot Plus strong solution #2504, Milipore (1:10 in Milipore-H2O) for 15 min shaking at room temperature, followed by blocking and detection with anti-GAPDH antibody (clone6C5, # MAB374, Millipore 1:10000 in 3% QuickBlocker/PBST) and secondary antibody (Peroxidase-conjugated goat anti-mouse IgG, Jackson Immunoresearch #115-035-003, 1:10000 in 3% BLOT Quickblocker/PBST).

Representative Blots from tumors of 2 animals per group treated with TPP-2090 3 mg/kg side by side with tumors from vehicle treated animals are shown in FIG. 23. Treatment with TPP-2090 leads to strong increase of total and phosphorylated Stat-1 levels as well as a strong activation of NF-kappaB2 as indicated by the appearance of the p52 fragment. Thus, the NF-kappaB2 as wells as Stat-1 pathways are activated by agonistic anti-TWEAKR antibodies in xenograft tumors and this activation is potentially involved in the anti-tumor activity of the corresponding antibodies.

TABLE 31
Protein sequences of the antibodies:

| | | | | | | | | | |
|---|---|---|---|---|---|---|---|---|---|
| SEQ ID NO: | SEQ ID NO: | SEQ ID | SEQ ID | SEQ ID | SEQ ID | SEQ ID | SEQ ID | SEQ ID | SEQ ID |
| IgG1 Light | IgG1 Heavy | NO: | NO: | NO: | NO: | NO: | NO: | NO: | NO: |
| Chain | Chain | L-CDR1 | L-CDR2 | L-CDR3 | H-CDR1 | H-CDR2 | H-CDR3 | VL Protein | VH Protein |

Antibodies of the invention:

| | | | | | | | | | | |
|---|---|---|---|---|---|---|---|---|---|---|
| TPP-2090 | 1 | 2 | 3 | 4 | 5 | 6 | 7 | 8 | 9 | 10 |
| TPP-2149 | 11 | 12 | 13 | 14 | 15 | 16 | 17 | 18 | 19 | 20 |
| TPP-2093 | 21 | 22 | 23 | 24 | 25 | 26 | 27 | 28 | 29 | 30 |
| TPP-2148 | 31 | 32 | 33 | 34 | 35 | 36 | 37 | 38 | 39 | 40 |
| TPP-2084 | 41 | 42 | 43 | 44 | 45 | 46 | 47 | 48 | 49 | 50 |
| TPP-2077 | 51 | 52 | 53 | 54 | 55 | 56 | 57 | 58 | 59 | 60 |
| TPP-1538 | 61 | 62 | 63 | 64 | 65 | 66 | 67 | 68 | 69 | 70 |
| TPP-883 | 71 | 72 | 73 | 74 | 75 | 76 | 77 | 78 | 79 | 80 |
| TPP-1854 | 81 | 82 | 83 | 84 | 85 | 86 | 87 | 88 | 89 | 90 |
| TPP-1853 | 91 | 92 | 93 | 94 | 95 | 96 | 97 | 98 | 99 | 100 |
| TPP-1857 | 101 | 102 | 103 | 104 | 105 | 106 | 107 | 108 | 109 | 110 |
| TPP-1858 | 111 | 112 | 113 | 114 | 115 | 116 | 117 | 118 | 119 | 120 |
| TPP-2658 | 1 | 213 | 3 | 4 | 5 | 6 | 7 | 8 | 9 | 10 |

Reference Antibodies

| | | |
|---|---|---|
| P3G5(TPP- | 121 | 122 |
| 2195) | | |
| 136.1(TPP- | 123 | 124 |
| 2194) | | |
| P4A8(TPP- | 125 | 126 |
| 1324) | | |
| PDL- | 127 | 128 |
| 192(TPP- | | |
| 1104) | | |
| 18.3.3(TPP- | 129 | 130 |
| 2193) | | |
| P2D3(TPP- | 131 | 132 |
| 2196) | | |

TABLE 32
DNA sequences of the antibodies of the invention

| | | |
|---|---|---|
| | SEQ ID NO: | SEQ ID NO: |
| Antibody | IgG1 Light Chain | IgG1 Heavy Chain |

Antibodies of the invention:

| | | |
|---|---|---|
| TPP-2090 | 177 | 178 |
| TPP-2149 | 179 | 180 |
| TPP-2093 | 181 | 182 |
| TPP-2148 | 183 | 184 |
| TPP-2084 | 185 | 186 |
| TPP-2077 | 187 | 188 |
| TPP-1538 | 189 | 190 |
| TPP-883 | 191 | 192 |
| TPP-1854 | 193 | 194 |
| TPP-1853 | 195 | 196 |
| TPP-1857 | 197 | 198 |
| TPP-1858 | 199 | 200 |

Reference antibodies:

| | | |
|---|---|---|
| P3G5(TPP-2195) | 201 | 202 |
| 136.1(TPP-2194) | 203 | 204 |
| P4A8(TPP-1324) | 205 | 206 |
| PDL-192(TPP-1104) | 207 | 208 |
| 18.3.3(TPP-2193) | 209 | 210 |
| P2D3(TPP-2196) | 211 | 212 |

### EXAMPLE 10

### Anti-Tumor Efficacy of Anti-TWEAKR Antibody TPP-2090 in Further Human Colorectal Cancer Models In Vivo

Animal studies were conducted as described in example 8 for further human colorectal cancer tumor cell lines Colo205 and LoVo and for further human colorectal cancer patient-derived models Co7553, Co5896, Co5676, Co5841, CXF 1103 and CXF 533. Standard dosing schedule was 10 mg/kg of TPP-2090 twice weekly for 4 weeks in monotherapy or in combination with regorafenib or the standard of cares (SoCs) irinotecan (5-15 mg/kg i.p., 4d on, 3d off), oxaliplatin (3-8 mg/kg i.p., twice weekly), 5-fluorouracil (50-100 mg/kg i.p., once weekly) and cetuximab (15 mg/kg i.p., twice weekly). TPP-2090 and cetuximab were formulated in PBS, which was also used as the vehicle in the control group, and the SoCs were formulated in 0.9% NaCl. The formulation of regorafenib is described in example 8.

The monotherapeutic efficacy of TPP-2090 in these human colorectal cancer patient-derived and cell line based models was moderate with final Tumor-to-Control (T/C) ratios in the range of 0.48-1.07. The combinations of TPP-2090 with SoCs (in particular 5-FU and irinotecan) resulted in significant additive and synergistic effects (see Table 33-35). In cases where the monotherapeutic efficacy of TPP-2090 in these models were limited more intense dosing schedules of anti-TWEAKR antibodies might be required to reach higher monotherapy efficacy, as has been shown in example 8 for colorectal cancer.

TABLE 33
Final Tumor-to-Control (T/C) ratios of colorectal cancer models treated
with TPP-2090 in monotherapy or combination with irinotecan and oxaliplatin

| | | Combination TPP-2090 with: | | | | | |
|---|---|---|---|---|---|---|---|
| | TPP-2090 | Irinotecan | | | Oxaliplatin | | |
| | MonoTx | | | Benefit of | | | Benefit |
| Tumormodel | Response | Mono | Combi | Combi | Mono | Combi | of Combi |
| Colo205 | 1.07 | 0.47 | 0.27 | synergistic | 0.89 | 1.20 | no benefit |
| | | | | effect | | | |
| LoVo | 1.25 | 0.52 | 0.64 | no benefit | 0.76 | 0.8 | no benefit |
| Co7553 | 0.79 | 0.11 | 0.15 | no benefit | 0.78 | 0.59 | additive |
| | | | | | | | effect |
| Co5896 | 0.91 | n.d. | n.d. | n.d. | n.d. | n.d. | n.d. |
| Co5676 (*) | 0.48 | 0.37 | 0.27 | additive | n.d. | n.d. | n.d. |
| | | | | effect | | | |
| Co5841 | 0.56 | 0.17 | 0.1 | no benefit | 0.81 | 0.58 | no benefit |
| CXF1103 | 0.87 | 0.66 | 0.39 | additive | 1.29 | 0.79 | no benefit |
| | | | | effect | | | |
| CXF533 | 0.88 | n.d. | n.d. | n.d. | 1.08 | 0.85 | no benefit |

T/C: tumor-to-control ratio based on final tumor weight after dissection or based on measurement of tumor area (*).
n.d.: not determined

TABLE 34
Final Tumor-to-Control (T/C) ratios of colorectal cancer models treated
with TPP-2090 in combination with 5-FU and regorafenib

| | Combination TPP-2090 with: | | | | | |
|---|---|---|---|---|---|---|
| | 5-FU | | | Regorafenib | | |
| | | | Benefit of | | | Benefit of |
| Tumormodel | Mono | Combi | Combi | Mono | Combi | Combi |
| Colo205 | n.d. | n.d. | n.d. | 0.42 | 0.46 | no benefit |
| LoVo | n.d. | n.d. | n.d. | 0.63 | 0.79 | no benefit |
| Co7553 | n.d. | n.d. | n.d. | n.d. | n.d. | n.d. |
| Co5896 | n.d. | n.d. | n.d. | 0.67 | 0.47 | synergistic |
| | | | | | | effect |
| Co5676 (*) | 0.45 | 0.20 | additive | n.d. | n.d. | n.d. |
| | | | effect | | | |
| Co5841 | 0.48 | 0.23 | additive | n.d. | n.d. | n.d. |
| | | | effect | | | |
| CXF1103 | 0.97 | 0.59 | synergistic | n.d. | n.d. | n.d. |
| | | | effect | | | |
| CXF533 | 0.28 | 0.34 | no benefit | n.d. | n.d. | n.d. |

T/C: tumor-to-control ratio based on final tumor weight after dissection or based on measurement of tumor area (*).
n.d.: not determined

TABLE 35
Tumor-to-Control (T/C) ratios of colorectal cancer models treated with
TPP-2090 in combination with cetuximab

| | Combination | | |
|---|---|---|---|
| | TPP-2090 with: | | |
| | Cetuximab | | |
| | | | Benefit of |
| Tumormodel | Mono | Combi | Combi |
| Colo205 | n.d. | n.d. | n.d. |
| LoVo | n.d. | n.d. | n.d. |
| Co7553 | n.d. | n.d. | n.d. |
| Co5896 | n.d. | n.d. | n.d. |
| Co5676(*) | 0.22 | 0.25 | no benefit |
| Co5841 | n.d. | n.d. | n.d. |
| CXF1103 | n.d. | n.d. | n.d. |
| CXF533 | 1.53 | 0.78 | additive |
| | | | effect |

T/C: tumor-to-control ratio based on final tumor weight after dissection or based on measurement of tumor area (*).
n.d.: not determined

### EXAMPLE 11

### Anti-Tumor Efficacy of Anti-TWEAKR Antibody TPP-2090 in Human Bladder Cancer Models In Vivo

Animal studies were conducted as described in example 8 for the human bladder cancer cell lines SCaBER and KU-19-19 and for the human bladder cancer patient-derived models BXF1352 and BXF1228. Standard dosing schedule was 10 mg/kg of TPP-2090 twice weekly for 4 weeks in monotherapy or in combination with the standard of cares (SoCs) gemcitabine (200 mg/kg i.p., once weekly) and cisplatin (3 mg/kg i.p., once weekly). TPP-2090 was formulated in PBS, which was also used as the vehicle in the control group, and the standard of cares (SoCs) were formulated in 0.9% NaCl. Strong monotherapeutic efficacy of TPP-2090 was found in SCaBER xenograft model. The combination of TPP-2090 in the human bladder cancer patient-derived bladder cancer models BXF1352 and BXF1228 with SoCs (Cisplatin and Gemcitabine) resulted in significant synergistic effects (see Table 36).). In cases where the monotherapeutic efficacy of TPP-2090 in these bladder cancer models were limited more intense dosing schedules of anti-TWEAKR antibodies might be required to reach higher monotherapy efficacy.

TABLE 36
Tumor-to-Control (T/C) ratios of human bladder cancer
models treated with TPP-2090 in monotherapy or
combination with cisplatin or gemcitabine

| | TPP- | Combination TPP-2090 with: | | | | | |
|---|---|---|---|---|---|---|---|
| | 2090 | | | | | | |
| | Mono | Oxaliplatin/Cisplatin | | | Gemcitabine | | |
| | Tx | | | Benefit | | | Benefit |
| Tumor- | Re- | | | of | | | of |
| model | sponse | Mono | Combi | Combi | Mono | Combi | Combi |
| SCaBER | 0.40 | 1.22 | 0.28 | synergis- | 0.85 | 0.42 | no |
| | | | | tic effect | | | benefit |
| Ku-19-19 | 0.86 | n.d. | n.d. | n.d. | 0.07 | n.d. | n.d. |
| BXF1352 | 0.93 | 0.85 | 0.4 | synergis- | n.d. | n.d. | n.d. |
| | | | | tic effect | | | |
| BXF1228 | 0.85 | 0.84 | 0.36 | synergis- | 0.93 | 0.46 | n.d. |
| | | | | tic effect | | | |

T/C: tumor-to-control ratio based on final tumor weight
n.d.: not determined

### EXAMPLE 12

### Anti-Tumor Efficacy of Anti-TWEAKR Antibody TPP-2090 in Further Human Cancer Models In Vivo

Animal studies were conducted as described in example 8 for further human cancer cell lines of different indications. Standard dosing schedule was 10 mg/kg of TPP-2090 twice weekly for 2-3 weeks in monotherapy. TPP-2090 was formulated in PBS, which was also used as the vehicle in the control group.

Strong monotherapeutic efficacy of TPP-2090 was found in SCC4 (head & neck cancer) and A375 (melanoma) xenografts, and moderate efficacy in BxPC3 (pancreatic cancer) xenografts (see Table 37). In cases where the monotherapeutic efficacy of TPP-2090 in certain xenograft models were limited (ACHN (renal cell cancer), PA-1 (ovarian cancer), NCI-292 (non-small cell lung cancer) and U87MG (glioblastoma)) more intense dosing schedules of anti-TWEAKR antibodies might be required to reach higher monotherapy efficacy.

TABLE 37
Tumor-to-control (T/C) ratios values of further human cancer models
treated with TPP-2090 in monotherapy

| Tumor | | Dose | Final T/C, TPP- |
|---|---|---|---|
| Model | Indication | schedule | 2090 |
| SCC4 | Head & neck cancer | Q4dx4 | 0.36 |
| A375 | Melanoma | Q4dx3 | 0.32 |
| Bx-PC3 | Pancreatic cancer | Q4dx3 | 0.50 |
| ACHN | Renal cell cancer | Q4dx6 | 0.78 (*) |
| NCI-H292 | Non-small cell lung | Single dose | 0.99 |
| | cancer | | |
| PA-1 | Ovarian cancer | Q4dx3 | 0.75 |
| U87MG | glioblastoma | Q4dx3 | 0.85 (*) |

T/C: tumor-to-control ratio based on final tumor weight after dissection or based on measurement of tumor area (*).

### EXAMPLE 13

### Further Mode of Action of Anti-TWEAKR Antibodies in Xenograft Models

To evaluate if the anti-tumor efficacy of TPP-2090 is dependent on antibody-dependent cellular cytotoxicity (ADCC) or agonistic activity alone is already sufficient, xenografts studies in SCID beige mice (Janvier) were conducted, and the aglycosylated variant of TPP-2090, namely TPP-2658, was investigated in NMRI nude mice.

Animal studies were conducted as described in example 8 in WiDr (human colorectal cancer) and SCaBER (human bladder cancer) xenografts in SCID beige mice with tumor growth of control groups comparable to those in NMRI nude mice of previous studies. Standard dosing schedule was 10 mg/kg of TPP-2090 i.v. (formulated in PBS) twice weekly for 2 weeks in monotherapy.

A similar strong monotherapeutic efficacy of TPP-2090 in NK-cell lacking SCID beige mice xenograft models (WiDr and SCaBER) was found as seen in NMRI nudes mice. This indicates an in vivo mode of action independent from ADCC (see Table 38).

TABLE 38
Tumor-to-Control (T/C) ratios of WiDr and SCaBER tumors in SCID
beige mice treated with TPP-2090 in monotherapy
(NMRI mice for comparison)

| | | | NMRI mice |
|---|---|---|---|
| | | SCID mice | final T/C |
| Tumor Model | Indication | Final T/C, TPP-2090 | TPP-2090 |
| WiDr | Colon cancer | 0.18 | 0.17 |
| SCaBER | Bladder cancer | 0.17 (*) | 0.40 |

T/C: tumor-to-control ratio based on final tumor weight after dissection or based on measurement of tumor area (*).

In vitro analysis showed that HT29 cell binding of TPP-2090 resulted in dose-dependent ADCC of target cells by NK92V effector cells while the aglycosylated TPP-2658 was not capable of inducing ADCC (Table 40). 1×10⁴ HT-29 target cells were dispensed and the tested antibodies were added in a final concentration of 25 μg/ml; 5 μg/ml; 1 μg/ml; 0.2 μg/ml and 0.04 μg/ml. After a preincubation time of 30 min effector cells were added (5×10⁴ NK92V effector cells). After 4 h incubation at 37° C. HT29 cell lysis was determined with the “Cytotoxicity Detection Kit—LDH (Roche)”, maximum release was obtained from cells solubilized in 1% Triton X-100, negative controls were not preincubated with an antibody. The following formula was used for calculation of % HT29 lysis: [Ext (sample)−Ext (negative)×100]/[Ext (Maximum release)−Ext (negative)]. TPP-2090 resulted in dose-dependent ADCC of target cells by NK92V effector cells and is dependent on N297 glycosylation.

Whereas in vivo a similar effect was found when an aglycosylated variant of TPP-2090, namely TPP-2658, was used in either a WiDr- or A375-xenograft model (see Table 39). The variant TPP-2658 showed equally strong monotherapeutic efficacy as the TPP-2090 in both models indicating an ADCC-independent mode of action.

TABLE 39
Tumor-to-Control (T/C) ratios of WiDr and A375-xenografts
treated with TPP-2658 (TPP-2090 for comparison)

| | | | Final T/C, | Final T/C |
|---|---|---|---|---|
| Tumor Model | Indication | Dose/schedule | TPP-2658 | TPP-2090 |
| WiDr | Colon cancer | 10 mg/kg, | 0.31 | 0.31 |
| | | q4dx3 | | |
| A375 | Melanoma | 10 mg/kg, | 0.45 | 0.32 |
| | | q4dx3 | | |

T/C: tumor-to-control ratio based on final tumor weight after dissection.

TABLE 40
In vitro ADCC assay with HT-29 target cells and NK92V effector
cells for testing antibody TPP-2090 (hIgG1) and TPP-2658
(aglycosylated counterpart of TPP-2090 - hIgG1 N297A):

| Antibody concentration | Lysis [%] for | Lysis [%] for TPP-2658 |
|---|---|---|
| [μg/ml] | TPP-2090 | (aglycosylated) |
| 25 | 16.8 | −1.1 |
| 5 | 16.4 | −1.3 |
| 1 | 15.1 | −1.3 |
| 0.2 | 9.8 | −1.4 |
| 0.04 | 6.3 | −0.8 |

1. An isolated anti-TWEAKR antibody or an antigen-binding fragment thereof, which specifically binds to the D at position 47 (D47) of TWEAKR as depicted in SEQ ID NO:169.

2. The antibody or an antigen binding fragment thereof according to claim 1 wherein the antibody is an agonistic antibody.

3. The antibody or an antigen binding fragment thereof according to claim 1, which comprises:
a variable heavy chain comprising:
(a) a heavy chain CDR1 encoded by an amino acid sequence comprising the formula PYPMX (SEQ ID NO: 171), wherein X is I or M;
(b) a heavy chain CDR2 encoded by an amino acid sequence comprising the formula YISPSGGXTHYADSVKG (SEQ ID NO: 172), wherein X is S or K; and
(c) a heavy chain CDR3 encoded by an amino acid sequence comprising the formula GGDTYFDYFDY (SEQ ID NO: 173);
and a variable light chain comprising:
(a) a light chain CDR1 encoded by an amino acid sequence comprising the formula RASQSISXYLN (SEQ ID NO: 174), wherein X is G or S;
(b) a light chain CDR2 encoded by an amino acid sequence comprising the formula XASSLQS (SEQ ID NO: 175), wherein X is Q, A, or N; and
(c) a light chain CDR3 encoded by an amino acid sequence comprising the formula QQSYXXPXIT (SEQ ID NO: 176), wherein X at position 5 is T or S, and X at position 6 is T or S, and X at position 8 is G, or F.

4. The antibody or an antigen binding fragment thereof according to claim 1 comprising:
a. a variable heavy chain comprising the variable heavy chain CDR1 sequence as presented by SEQ ID NO: 6, the variable heavy chain CDR2 sequence as presented by SEQ ID NO: 7, and the variable heavy chain CDR3 sequence as presented by SEQ ID NO: 8, and
a variable light chain comprising the variable light chain CDR1 sequence presented by SEQ ID NO: 3, the variable light chain CDR2 sequence presented by SEQ ID NO: 4, and the variable light chain CDR3 sequence presented by SEQ ID NO: 5, or
b. a variable heavy chain comprising the variable heavy chain CDR1 sequence as presented by SEQ ID NO: 16, the variable heavy chain CDR2 sequence as presented by SEQ ID NO: 17, the variable heavy chain CDR3 sequence as presented by SEQ ID NO:18, and
a variable light chain comprising the variable light chain CDR1 sequence presented by SEQ ID NO: 13, the variable light chain CDR2 sequence presented by SEQ ID NO: 14, and the variable light chain CDR3 sequence presented by SEQ ID NO:15, or
c. a variable heavy chain comprising the variable heavy chain CDR1 sequence as presented by SEQ ID NO: 26, the variable heavy chain CDR2 sequence as presented by SEQ ID NO: 27, the variable heavy chain CDR3 sequence as presented by SEQ ID NO:28, and
a variable light chain comprising the variable light chain CDR1 sequence presented by SEQ ID NO: 23, the variable light chain CDR2 sequence presented by SEQ ID NO: 24, and the variable light chain CDR3 sequence presented by SEQ ID NO:25, or
d. a variable heavy chain comprising the variable heavy chain CDR1 sequence as presented by SEQ ID NO: 36, the variable heavy chain CDR2 sequence as presented by SEQ ID NO: 37, the variable heavy chain CDR3 sequence as presented by SEQ ID NO:38, and
a variable light chain comprising the variable light chain CDR1 sequence presented by SEQ ID NO: 33, the variable light chain CDR2 sequence presented by SEQ ID NO: 34, and the variable light chain CDR3 sequence presented by SEQ ID NO:35, or
e. a variable heavy chain comprising the variable heavy chain CDR1 sequence as presented by SEQ ID NO: 46, the variable heavy chain CDR2 sequence as presented by SEQ ID NO: 47, the variable heavy chain CDR3 sequence as presented by SEQ ID NO:48, and
a variable light chain comprising the variable light chain CDR1 sequence presented by SEQ ID NO: 43, the variable light chain CDR2 sequence presented by SEQ ID NO: 44, and the variable light chain CDR3 sequence presented by SEQ ID NO:45, or
f. a variable heavy chain comprising the variable heavy chain CDR1 sequence as presented by SEQ ID NO: 56, the variable heavy chain CDR2 sequence as presented by SEQ ID NO: 57, the variable heavy chain CDR3 sequence as presented by SEQ ID NO:58, and
a variable light chain comprising the variable light chain CDR1 sequence presented by SEQ ID NO: 53, the variable light chain CDR2 sequence presented by SEQ ID NO: 54, and the variable light chain CDR3 sequence presented by SEQ ID NO:55, or
g. a variable heavy chain comprising the variable heavy chain CDR1 sequence as presented by SEQ ID NO: 66, the variable heavy chain CDR2 sequence as presented by SEQ ID NO: 67, the variable heavy chain CDR3 sequence as presented by SEQ ID NO:68, and
a variable light chain comprising the variable light chain CDR1 sequence presented by SEQ ID NO: 63, the variable light chain CDR2 sequence presented by SEQ ID NO: 64, and the variable light chain CDR3 sequence presented by SEQ ID NO:65, or
h. a variable heavy chain comprising the variable heavy chain CDR1 sequence as presented by SEQ ID NO: 76, the variable heavy chain CDR2 sequence as presented by SEQ ID NO: 77, the variable heavy chain CDR3 sequence as presented by SEQ ID NO:78, and
a variable light chain comprising the variable light chain CDR1 sequence presented by SEQ ID NO: 73, the variable light chain CDR2 sequence presented by SEQ ID NO: 74, and the variable light chain CDR3 sequence presented by SEQ ID NO:75, or
i. a variable heavy chain comprising the variable heavy chain CDR1 sequence as presented by SEQ ID NO: 86, the variable heavy chain CDR2 sequence as presented by SEQ ID NO: 87, the variable heavy chain CDR3 sequence as presented by SEQ ID NO:88, and
a variable light chain comprising the variable light chain CDR1 sequence presented by SEQ ID NO: 83, the variable light chain CDR2 sequence presented by SEQ ID NO: 84, and the variable light chain CDR3 sequence presented by SEQ ID NO:85, or
j. a variable heavy chain comprising the variable heavy chain CDR1 sequence as presented by SEQ ID NO: 96, the variable heavy chain CDR2 sequence as presented by SEQ ID NO: 97, the variable heavy chain CDR3 sequence as presented by SEQ ID NO:98, and
a variable light chain comprising the variable light chain CDR1 sequence presented by SEQ ID NO: 93, the variable light chain CDR2 sequence presented by SEQ ID NO: 94, and the variable light chain CDR3 sequence presented by SEQ ID NO:95, or
k. a variable heavy chain comprising the variable heavy chain CDR1 sequence as presented by SEQ ID NO: 106, the variable heavy chain CDR2 sequence as presented by SEQ ID NO: 107, the variable heavy chain CDR3 sequence as presented by SEQ ID NO:108, and
a variable light chain comprising the variable light chain CDR1 sequence presented by SEQ ID NO: 103, the variable light chain CDR2 sequence presented by SEQ ID NO: 104, and the variable light chain CDR3 sequence presented by SEQ ID NO:105 or
l. a variable heavy chain comprising the variable heavy chain CDR1 sequence as presented by SEQ ID NO: 116, the variable heavy chain CDR2 sequence as presented by SEQ ID NO: 117, the variable heavy chain CDR3 sequence as presented by SEQ ID NO:118, and
a variable light chain comprising the variable light chain CDR1 sequence presented by SEQ ID NO: 113, the variable light chain CDR2 sequence presented by SEQ ID NO: 114, and the variable light chain CDR3 sequence presented by SEQ ID NO:115.

5. The antibody or antigen-binding fragment thereof according to claim 1 comprising:
a. a variable heavy chain sequence as presented by SEQ ID NO:10 and a variable light chain sequences as presented by SEQ ID NO:9, or
b. a variable heavy chain sequence as presented by SEQ ID NO:20 and a variable light chain sequences as presented by SEQ ID NO:19, or
c. a variable heavy chain sequence as presented by SEQ ID NO:30 and a variable light chain sequences as presented by SEQ ID NO:29, or
d. a variable heavy chain sequence as presented by SEQ ID NO:40 and a variable light chain sequences as presented by SEQ ID NO:39, or
e. a variable heavy chain sequence as presented by SEQ ID NO:50 and a variable light chain sequences as presented by SEQ ID NO:49, or
f. a variable heavy chain sequence as presented by SEQ ID NO:60 and a variable light chain sequences as presented by SEQ ID NO:59, or
g. a variable heavy chain sequence as presented by SEQ ID NO:70 and a variable light chain sequences as presented by SEQ ID NO:69, or
h. a variable heavy chain sequence as presented by SEQ ID NO:80 and a variable light chain sequences as presented by SEQ ID NO:79, or
i. a variable heavy chain sequence as presented by SEQ ID NO:90 and a variable light chain sequences as presented by SEQ ID NO:89, or
j. a variable heavy chain sequence as presented by SEQ ID NO:100 and a variable light chain sequences as presented by SEQ ID NO:99, or
k. a variable heavy chain sequence as presented by SEQ ID NO:110 and a variable light chain sequences as presented by SEQ ID NO:109, or
l. a variable heavy chain sequence as presented by SEQ ID NO:120 and a variable light chain sequences as presented by SEQ ID NO:119.

6. The antibody according to claim 1, which is an IgG antibody.

7. The antibody according to claim 1 comprising:
a. a heavy chain sequence as presented by SEQ ID NO:2 and a light chain sequences as presented by SEQ ID NO:1, or
b. a heavy chain sequence as presented by SEQ ID NO:12 and a light chain sequences as presented by SEQ ID NO:11, or
c. a heavy chain sequence as presented by SEQ ID NO:22 and a light chain sequences as presented by SEQ ID NO:21, or
d. a heavy chain sequence as presented by SEQ ID NO:32 and a light chain sequences as presented by SEQ ID NO:31, or
e. a heavy chain sequence as presented by SEQ ID NO:42 and a light chain sequences as presented by SEQ ID NO:41, or
f. a heavy chain sequence as presented by SEQ ID NO:52 and a light chain sequences as presented by SEQ ID NO:51, or
g. a heavy chain sequence as presented by SEQ ID NO:62 and a light chain sequences as presented by SEQ ID NO:61, or
h. a heavy chain sequence as presented by SEQ ID NO:72 and a light chain sequences as presented by SEQ ID NO:71, or
i. a heavy chain sequence as presented by SEQ ID NO:82 and a light chain sequences as presented by SEQ ID NO:81, or
j. a heavy chain sequence as presented by SEQ ID NO:92 and a light chain sequences as presented by SEQ ID NO:91, or
k. a heavy chain sequence as presented by SEQ ID NO:102 and a light chain sequences as presented by SEQ ID NO:101, or
l. a heavy chain sequence as presented by SEQ ID NO:112 and a light chain sequences as presented by SEQ ID NO:111.

8. The antigen-binding fragment according to claim 1, which is an scFv, Fab, Fab′ fragment or a F(ab′)2 fragment.

9. The antibody or antigen-binding fragment according to claim 1, which is a monoclonal antibody or antigen-binding fragment thereof.

10. The antibody or antigen-binding fragment according to claim 1, which is a human, humanized or chimeric antibody or antigen-binding fragment.

11. An antibody-drug conjugate, comprising an antibody or antigen binding fragment thereof according to claim 1.

12. An isolated nucleic acid sequence that encodes the antibody or antigen-binding fragment according to claim 1.

13. A vector comprising a nucleic acid sequence according to claim 12.

14. An isolated cell expressing an antibody or antigen-binding fragment according to claim 1.

15. An isolated cell according to claim 14, wherein said cell is a prokaryotic or an eukaryotic cell.

16. A method of producing an antibody or antigen-binding fragment according to claim 1 comprising culturing of a prokaryotic or an eukaryotic cell expressing said antibody or antigen-binding fragment and purification of the antibody or antigen-binding fragment.

17. An antibody or antigen-binding fragment according to claim 1 for use as a medicament.

18. An antibody or antigen antigen-binding fragment according to claim 1 for use as a diagnostic agent.

19. An antibody or antigen-binding fragment according to claim 1 for use as a medicament for the treatment of cancer.

20. A pharmaceutical composition comprising an antibody or antigen-binding fragment according to claim 1.

21. A combination of a pharmaceutical composition according to claim 20 and one or more therapeutically active compounds.

22. A method for treating a disorder or condition associated with the undesired presence of TWEAKR, comprising administering to a subject in need thereof an effective amount of the pharmaceutical composition according to claim 20.


SEQUENCE LISTING (213 sequences)

SEQ ID NO: 1 - protein, 215 aa, Homo Sapiens
DIQMTQSPSSLSASVGDRVTITCRASQSISGYLNWYQQKPGKAPKLLIYQASSLQSGVPSRFSGSGSGTDFTLTISSLQPEDFATYYCQQSYTSPFITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, Homo Sapiens
EVQLLESGGGLVQPGGSLRLSCAASGFTFSPYPMIWVRQAPGKGLEWVSYISPSGGSTHYADSVKGRFTISRDNSKNTLYLQMNSLRAEDTAVYYCARGGDTYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 11 aa, Homo Sapiens
RASQSISGYLN

SEQ ID NO: 4 - protein, 7 aa, Homo Sapiens
QASSLQS

SEQ ID NO: 5 - protein, 10 aa, Homo Sapiens
QQSYTSPFIT

SEQ ID NO: 6 - protein, 5 aa, Homo Sapiens
PYPMI

SEQ ID NO: 7 - protein, 17 aa, Homo Sapiens
YISPSGGSTHYADSVKG

SEQ ID NO: 8 - protein, 11 aa, Homo Sapiens
GGDTYFDYFDY

SEQ ID NO: 9 - protein, 108 aa, Homo Sapiens
DIQMTQSPSSLSASVGDRVTITCRASQSISGYLNWYQQKPGKAPKLLIYQASSLQSGVPSRFSGSGSGTDFTLTISSLQPEDFATYYCQQSYTSPFITFGQGTKVEIK

SEQ ID NO: 10 - protein, 121 aa, Homo Sapiens
EVQLLESGGGLVQPGGSLRLSCAASGFTFSPYPMIWVRQAPGKGLEWVSYISPSGGSTHYADSVKGRFTISRDNSKNTLYLQMNSLRAEDTAVYYCARGGDTYFDYFDYWGQGTLVTVSSA

SEQ ID NO: 11 - protein, 215 aa, Homo Sapiens
DIQMTQSPATLSASVGDRVTITCRASQSISGYLNWYQQKPGKAPKLLIYQASSLQSGVPSRFSGSGSGTDFTLTISSLQPEDFATYYCQQSYTSPFITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 12 - protein, 449 aa, Homo Sapiens
EVQLLESGGGLVQPGGSLRLSCAASGFTFSPYPMIWVRQAPGKGLEWVSYISPSGGKTHYADSVKGRFTISRDNSKNTLYLQMNSLRAEDTAVYYCARGGDTYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 11 aa, Homo Sapiens
RASQSISGYLN

SEQ ID NO: 14 - protein, 7 aa, Homo Sapiens
QASSLQS

SEQ ID NO: 15 - protein, 10 aa, Homo Sapiens
QQSYTSPFIT

SEQ ID NO: 16 - protein, 5 aa, Homo Sapiens
PYPMI

SEQ ID NO: 17 - protein, 17 aa, Homo Sapiens
YISPSGGKTHYADSVKG

SEQ ID NO: 18 - protein, 11 aa, Homo Sapiens
GGDTYFDYFDY

SEQ ID NO: 19 - protein, 108 aa, Homo Sapiens
DIQMTQSPATLSASVGDRVTITCRASQSISGYLNWYQQKPGKAPKLLIYQASSLQSGVPSRFSGSGSGTDFTLTISSLQPEDFATYYCQQSYTSPFITFGPGTKVEIK

SEQ ID NO: 20 - protein, 121 aa, Homo Sapiens
EVQLLESGGGLVQPGGSLRLSCAASGFTFSPYPMIWVRQAPGKGLEWVSYISPSGGKTHYADSVKGRFTISRDNSKNTLYLQMNSLRAEDTAVYYCARGGDTYFDYFDYWGQGTLVTVSSA

SEQ ID NO: 21 - protein, 215 aa, Homo Sapiens
DIQMTQSPSSLSASVGDRVTITCRASQSISSYLNWYQQKPGKAPKLLIYQASSLQSGVPSRFSGSGSGTDFTLTISSLQPEDFATYYCQQSYTSPFITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 449 aa, Homo Sapiens
EVQLLESGGGLVQPGGSLRLSCAASGFTFSPYPMMWVRQAPGKGLEWVSYISPSGGSTHYADSVKGRFTISRDNSKNTLYLQMNSLRAEDTAVYYCARGGDTYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 11 aa, Homo Sapiens
RASQSISSYLN

SEQ ID NO: 24 - protein, 7 aa, Homo Sapiens
QASSLQS

SEQ ID NO: 25 - protein, 10 aa, Homo Sapiens
QQSYTSPFIT

SEQ ID NO: 26 - protein, 5 aa, Homo Sapiens
PYPMM

SEQ ID NO: 27 - protein, 17 aa, Homo Sapiens
YISPSGGSTHYADSVKG

SEQ ID NO: 28 - protein, 11 aa, Homo Sapiens
GGDTYFDYFDY

SEQ ID NO: 29 - protein, 108 aa, Homo Sapiens
DIQMTQSPSSLSASVGDRVTITCRASQSISSYLNWYQQKPGKAPKLLIYQASSLQSGVPSRFSGSGSGTDFTLTISSLQPEDFATYYCQQSYTSPFITFGQGTKVEIK

SEQ ID NO: 30 - protein, 121 aa, Homo Sapiens
EVQLLESGGGLVQPGGSLRLSCAASGFTFSPYPMMWVRQAPGKGLEWVSYISPSGGSTHYADSVKGRFTISRDNSKNTLYLQMNSLRAEDTAVYYCARGGDTYFDYFDYWGQGTLVTVSSA

SEQ ID NO: 31 - protein, 215 aa, Homo Sapiens
DIQMTQSPATLSASVGDRVTITCRASQSISSYLNWYQQKPGKAPKLLIYQASSLQSGVPSRFSGSGSGTDFTLTISSLQPEDFATYYCQQSYTSPFITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 32 - protein, 449 aa, Homo Sapiens
EVQLLESGGGLVQPGGSLRLSCAASGFTFSPYPMMWVRQAPGKGLEWVSYISPSGGKTHYADSVKGRFTISRDNSKNTLYLQMNSLRAEDTAVYYCARGGDTYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 11 aa, Homo Sapiens
RASQSISSYLN

SEQ ID NO: 34 - protein, 7 aa, Homo Sapiens
QASSLQS

SEQ ID NO: 35 - protein, 10 aa, Homo Sapiens
QQSYTSPFIT

SEQ ID NO: 36 - protein, 5 aa, Homo Sapiens
PYPMM

SEQ ID NO: 37 - protein, 17 aa, Homo Sapiens
YISPSGGKTHYADSVKG

SEQ ID NO: 38 - protein, 11 aa, Homo Sapiens
GGDTYFDYFDY

SEQ ID NO: 39 - protein, 108 aa, Homo Sapiens
DIQMTQSPATLSASVGDRVTITCRASQSISSYLNWYQQKPGKAPKLLIYQASSLQSGVPSRFSGSGSGTDFTLTISSLQPEDFATYYCQQSYTSPFITFGPGTKVEIK

SEQ ID NO: 40 - protein, 121 aa, Homo Sapiens
EVQLLESGGGLVQPGGSLRLSCAASGFTFSPYPMMWVRQAPGKGLEWVSYISPSGGKTHYADSVKGRFTISRDNSKNTLYLQMNSLRAEDTAVYYCARGGDTYFDYFDYWGQGTLVTVSSA

SEQ ID NO: 41 - protein, 215 aa, Homo Sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPGITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 449 aa, Homo Sapiens; identical to SEQ ID NO: 22

SEQ ID NO: 43 - protein, 11 aa, Homo Sapiens
RASQSISSYLN

SEQ ID NO: 44 - protein, 7 aa, Homo Sapiens
AASSLQS

SEQ ID NO: 45 - protein, 10 aa, Homo Sapiens
QQSYSTPGIT

SEQ ID NO: 46 - protein, 5 aa, Homo Sapiens
PYPMM

SEQ ID NO: 47 - protein, 17 aa, Homo Sapiens
YISPSGGSTHYADSVKG

SEQ ID NO: 48 - protein, 11 aa, Homo Sapiens
GGDTYFDYFDY

SEQ ID NO: 49 - protein, 108 aa, Homo Sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPGITFGQGTKVEIK

SEQ ID NO: 50 - protein, 121 aa, Homo Sapiens; identical to SEQ ID NO: 30

SEQ ID NO: 51 - protein, 215 aa, Homo Sapiens
DIQMTQSPATLSASVGDRVTITCRASQSISSYLNWYQQKPGKAPKLLIYAASSLQSGVPSRFSGSGSGTDFTLTISSLQPEDFATYYCQQSYSSPGITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 52 - protein, 449 aa, Homo Sapiens; identical to SEQ ID NO: 32

SEQ ID NO: 53 - protein, 11 aa, Homo Sapiens
RASQSISSYLN

SEQ ID NO: 54 - protein, 7 aa, Homo Sapiens
AASSLQS

SEQ ID NO: 55 - protein, 10 aa, Homo Sapiens
QQSYSSPGIT

SEQ ID NO: 56 - protein, 5 aa, Homo Sapiens
PYPMM

SEQ ID NO: 57 - protein, 17 aa, Homo Sapiens
YISPSGGKTHYADSVKG

SEQ ID NO: 58 - protein, 11 aa, Homo Sapiens
GGDTYFDYFDY

SEQ ID NO: 59 - protein, 108 aa, Homo Sapiens
DIQMTQSPATLSASVGDRVTITCRASQSISSYLNWYQQKPGKAPKLLIYAASSLQSGVPSRFSGSGSGTDFTLTISSLQPEDFATYYCQQSYSSPGITFGPGTKVEIK

SEQ ID NO: 60 - protein, 121 aa, Homo Sapiens; identical to SEQ ID NO: 40

SEQ ID NO: 61 - protein, 217 aa, Homo Sapiens
AQDIQMTQSPATLSASVGDRVTITCRASQSISSYLNWYQQKPGKAPKLLIYAASSLQSGVPSRFSGSGSGTDFTLTISSLQPEDFATYYCQQSYSSPGITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 62 - protein, 449 aa, Homo Sapiens; identical to SEQ ID NO: 32

SEQ ID NO: 63 - protein, 11 aa, Homo Sapiens
RASQSISSYLN

SEQ ID NO: 64 - protein, 7 aa, Homo Sapiens
AASSLQS

SEQ ID NO: 65 - protein, 10 aa, Homo Sapiens
QQSYSSPGIT

SEQ ID NO: 66 - protein, 5 aa, Homo Sapiens
PYPMM

SEQ ID NO: 67 - protein, 17 aa, Homo Sapiens
YISPSGGKTHYADSVKG

SEQ ID NO: 68 - protein, 11 aa, Homo Sapiens
GGDTYFDYFDY

SEQ ID NO: 69 - protein, 110 aa, Homo Sapiens
AQDIQMTQSPATLSASVGDRVTITCRASQSISSYLNWYQQKPGKAPKLLIYAASSLQSGVPSRFSGSGSGTDFTLTISSLQPEDFATYYCQQSYSSPGITFGPGTKVEIK

SEQ ID NO: 70 - protein, 121 aa, Homo Sapiens; identical to SEQ ID NO: 40

SEQ ID NO: 71 - protein, 217 aa, Homo Sapiens; identical to SEQ ID NO: 61

SEQ ID NO: 72 - protein, 449 aa, Homo Sapiens
EVQLLESGGGLVQPGGSLRLSCAASGFTFSPYPMMWVRQAPGKGLEWVSYISPSGGKTHYADSVKGRFTISRDNSKNTLYLQMNSLRAEDTAVYYCARGGDGYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 11 aa, Homo Sapiens
RASQSISSYLN

SEQ ID NO: 74 - protein, 7 aa, Homo Sapiens
AASSLQS

SEQ ID NO: 75 - protein, 10 aa, Homo Sapiens
QQSYSSPGIT

SEQ ID NO: 76 - protein, 5 aa, Homo Sapiens
PYPMM

SEQ ID NO: 77 - protein, 17 aa, Homo Sapiens
YISPSGGKTHYADSVKG

SEQ ID NO: 78 - protein, 11 aa, Homo Sapiens
GGDGYFDYFDY

SEQ ID NO: 79 - protein, 110 aa, Homo Sapiens; identical to SEQ ID NO: 69

SEQ ID NO: 80 - protein, 121 aa, Homo Sapiens
EVQLLESGGGLVQPGGSLRLSCAASGFTFSPYPMMWVRQAPGKGLEWVSYISPSGGKTHYADSVKGRFTISRDNSKNTLYLQMNSLRAEDTAVYYCARGGDGYFDYFDYWGQGTLVTVSSA

SEQ ID NO: 81 - protein, 217 aa, Homo Sapiens
AQDIQMTQSPATLSASVGDRVTITCRASQSISGYLNWYQQKPGKAPKLLIYNASSLQSGVPSRFSGSGSGTDFTLTISSLQPEDFATYYCQQSYTSPFITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 82 - protein, 449 aa, Homo Sapiens; identical to SEQ ID NO: 12

SEQ ID NO: 83 - protein, 11 aa, Homo Sapiens
RASQSISGYLN

SEQ ID NO: 84 - protein, 7 aa, Homo Sapiens
NASSLQS

SEQ ID NO: 85 - protein, 10 aa, Homo Sapiens
QQSYTSPFIT

SEQ ID NO: 86 - protein, 5 aa, Homo Sapiens
PYPMI

SEQ ID NO: 87 - protein, 17 aa, Homo Sapiens
YISPSGGKTHYADSVKG

SEQ ID NO: 88 - protein, 11 aa, Homo Sapiens
GGDTYFDYFDY

SEQ ID NO: 89 - protein, 110 aa, Homo Sapiens
AQDIQMTQSPATLSASVGDRVTITCRASQSISGYLNWYQQKPGKAPKLLIYNASSLQSGVPSRFSGSGSGTDFTLTISSLQPEDFATYYCQQSYTSPFITFGPGTKVEIK

SEQ ID NO: 90 - protein, 121 aa, Homo Sapiens; identical to SEQ ID NO: 20

SEQ ID NO: 91 - protein, 217 aa, Homo Sapiens
AQDIQMTQSPATLSASVGDRVTITCRASQSISSYLNWYQQKPGKAPKLLIYNASSLQSGVPSRFSGSGSGTDFTLTISSLQPEDFATYYCQQSYTSPGITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 92 - protein, 449 aa, Homo Sapiens; identical to SEQ ID NO: 32

SEQ ID NO: 93 - protein, 11 aa, Homo Sapiens
RASQSISSYLN

SEQ ID NO: 94 - protein, 7 aa, Homo Sapiens
NASSLQS

SEQ ID NO: 95 - protein, 10 aa, Homo Sapiens
QQSYTSPGIT

SEQ ID NO: 96 - protein, 5 aa, Homo Sapiens
PYPMM

SEQ ID NO: 97 - protein, 17 aa, Homo Sapiens
YISPSGGKTHYADSVKG

SEQ ID NO: 98 - protein, 11 aa, Homo Sapiens
GGDTYFDYFDY

SEQ ID NO: 99 - protein, 110 aa, Homo Sapiens
AQDIQMTQSPATLSASVGDRVTITCRASQSISSYLNWYQQKPGKAPKLLIYNASSLQSGVPSRFSGSGSGTDFTLTISSLQPEDFATYYCQQSYTSPGITFGPGTKVEIK

SEQ ID NO: 100 - protein, 121 aa, Homo Sapiens; identical to SEQ ID NO: 40

SEQ ID NO: 101 - protein, 217 aa, Homo Sapiens
AQDIQMTQSPATLSASVGDRVTITCRASQSISGYLNWYQQKPGKAPKLLIYNASSLQSGVPSRFSGSGSGTDFTLTISSLQPEDFATYYCQQSYTSPGITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 102 - protein, 449 aa, Homo Sapiens; identical to SEQ ID NO: 32

SEQ ID NO: 103 - protein, 11 aa, Homo Sapiens
RASQSISGYLN

SEQ ID NO: 104 - protein, 7 aa, Homo Sapiens
NASSLQS

SEQ ID NO: 105 - protein, 10 aa, Homo Sapiens
QQSYTSPGIT

SEQ ID NO: 106 - protein, 5 aa, Homo Sapiens
PYPMM

SEQ ID NO: 107 - protein, 17 aa, Homo Sapiens
YISPSGGKTHYADSVKG

SEQ ID NO: 108 - protein, 11 aa, Homo Sapiens
GGDTYFDYFDY

SEQ ID NO: 109 - protein, 110 aa, Homo Sapiens
AQDIQMTQSPATLSASVGDRVTITCRASQSISGYLNWYQQKPGKAPKLLIYNASSLQSGVPSRFSGSGSGTDFTLTISSLQPEDFATYYCQQSYTSPGITFGPGTKVEIK

SEQ ID NO: 110 - protein, 121 aa, Homo Sapiens; identical to SEQ ID NO: 40

SEQ ID NO: 111 - protein, 217 aa, Homo Sapiens
AQDIQMTQSPATLSASVGDRVTITCRASQSISSYLNWYQQKPGKAPKLLIYNASSLQSGVPSRFSGSGSGTDFTLTISSLQPEDFATYYCQQSYTSPFITFGPGTKVEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 112 - protein, 449 aa, Homo Sapiens; identical to SEQ ID NO: 32

SEQ ID NO: 113 - protein, 11 aa, Homo Sapiens
RASQSISSYLN

SEQ ID NO: 114 - protein, 7 aa, Homo Sapiens
NASSLQS

SEQ ID NO: 115 - protein, 10 aa, Homo Sapiens
QQSYTSPFIT

SEQ ID NO: 116 - protein, 5 aa, Homo Sapiens
PYPMM

SEQ ID NO: 117 - protein, 17 aa, Homo Sapiens
YISPSGGKTHYADSVKG

SEQ ID NO: 118 - protein, 11 aa, Homo Sapiens
GGDTYFDYFDY

SEQ ID NO: 119 - protein, 110 aa, Homo Sapiens
AQDIQMTQSPATLSASVGDRVTITCRASQSISSYLNWYQQKPGKAPKLLIYNASSLQSGVPSRFSGSGSGTDFTLTISSLQPEDFATYYCQQSYTSPFITFGPGTKVEIK

SEQ ID NO: 120 - protein, 121 aa, Homo Sapiens; identical to SEQ ID NO: 40

SEQ ID NO: 121 - protein, 218 aa, Mus Musculus
DIVLTQSPASLAVSLGQRATISCRANKSVSTSSYSYMHWYQQKPGQPPKLLIKYASNLESGVPARFSGSGSGTDFILNIHPVEEEDAATYYCQHSRELPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 122 - protein, 450 aa, Mus Musculus
QVQLQQSGPEVVRPGVSVKISCKGSGYTFTDYGIHWVKQSHAKSLEWIGVISTYNGYTNYNQKFKGKATMTVDKSSSTAYMELARLTSEDSAIYYCARAYYGNLYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 123 - protein, 218 aa, Mus Musculus
DIVLTQSPASLTVSLGQRATISCRASQSVSTSSYSYMQWYQQRPGQPPKLLIKYATNLDSGVPARFSGSGSGTDFTLNIHPVEEEDAATYYCQHSWEIPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 124 - protein, 448 aa, Mus Musculus
EVKLEESGGGLVQPGGSMKLSCVASGFTFNNYWMSWVRQSPEKGLEWLAEIRLKSDNYATHYAESVKGKFTISRDDSKSRLYLQMNNLRAENTGIYYCTGGFADYFDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 125 - protein, 218 aa, Homo Sapiens
DIVLTQSPASLAVSLGQRATISCRASKSVSTSSYSYMHWYQQKPGQPPKLLIKYASNLESGVPARFSGSGSGTDFSLNIHPMEEDDTAMYFCQHSRELPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 126 - protein, 450 aa, Homo Sapiens
QVQLVQSGAEVKKPGASVKVSCKGSGYTFTDYGMHWVRQAPGQGLEWMGVISTYNGYTNYNQKFKGRVTMTVDKSTSTAYMELRSLRSDDTAVYYCARAYYGNLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 218 aa, Homo Sapiens
DIQMTQSPSSLSASVGDRVTITCRASQSVSTSSYSYMHWYQQKPGKAPKLLIKYASNLESGVPSRFSGSGSGTDFTLTISSLQPEDFATYYCQHSWEIPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 449 aa, Homo Sapiens
QVELVESGGGLVQPGGSLRLSCAASGFTFSSYWMSWVRQAPGKGLEWVAEIRLKSDNYATHYAESVKGRFTISRDDSKNSLYLQMNSLRAEDTAVYYCTGYYADAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 218 aa, Mus Musculus
DIVLTQSPASLAVSLGQRATISCKASQSVSTSTYSYMQWYQQRPGQSPKLLIKYASKLDSGVPARFSGSGSGTDFTLNIHPVEEEDTATYYCQHSWELPYTFGGGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 130 - protein, 448 aa, Mus Musculus
EVKLGESGGGLVQPGGSMKLSCVASGFPFTKYWMNWVRQSPEKGLEWVAEIRLKSDNYATHYAESAKGRFTISRDDSRSSVYLQMNNLRAEDTAIYYCSPTYADTMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 131 - protein, 218 aa, Mus Musculus
DIVLTQSPASLAVSLGQRATISCRASKSVSTSSYSYMHWYQQKPGQPPKLLIKYTSNLESGVPARFSGSGSGTDFILNIHPVEEEDAATYYCQHSRELPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 132 - protein, 451 aa, Mus Musculus
QVSLKESGPGILQPSQTLSLTCSFSGFSLSTSGMGVSWIRQPSGKGLEWLAHIYWDDDKRYNPSLKSRLTISKDTSRNQVFLKITSVDTADTATYYCARRGPDYYGYYPMDYWGQGTSVTVSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPG

SEQ ID NO: 133 - protein, 282 aa, Macaca fascicularis
EQAPGTAPCSHGSSWSADLDKCMDCASCRARPHSDFCLGCSAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 282 aa, Rattus norvegicus
EQAPGNAPCSSGSSWSADLDKCMDCASCPARPHSDFCLGCAAAPPAHFRMLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 282 aa, Sus scrofa
ERVPGTTPCSRGSSWSADLDKCMDCASCPARPHSDFCLGCAAAPPASFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 282 aa, Canis lupus
ERVPGTTPCPRGSSWSADLDKCMDCASCRARPHSDFCLGCTAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 282 aa, Mus Musculus
EQAPGTSPCSSGSSWSADLDKCMDCASCPARPHSDFCLGCAAAPPAHFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 282 aa, Homo Sapiens
EQAPGTAPCSRGSSWSADLDKCMDCASCRARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 296 aa, Homo Sapiens
EQAPGTAPCSRGSSWSADLDKCMDCASCRARPHSDFCLGCAAAPPAPFRLLWPIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 140 - protein, 278 aa, Homo Sapiens
APCSRGSSWSADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 141 - protein, 47 aa, Homo Sapiens
EQAPGTAPCSRGSSWSADLDKCMDCASCRARPHSDFCLGCAHHHHHH

SEQ ID NO: 142 - protein, 282 aa, Homo Sapiens
EQAPGTAPCSRGSSWSADLDKCMDCASCRARPHSDFCQGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 282 aa, Homo Sapiens
EQAPGTAPCSRGSSWSADLDKCMDCASCRARPKSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 282 aa, Homo Sapiens
EQAPGTAPCSRGSSWSADLDKCMDCASCPARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 282 aa, Homo Sapiens
EQAPGTAPCSRGSSWSADLDKCADCASCRARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 282 aa, Homo Sapiens
EQAPGTAPCSRGSSASADLDKCMDCASCRARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 282 aa, Homo Sapiens
EQAPGTAPCSRGRSWSADLDKCMDCASCRARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 282 aa, Homo Sapiens
EQAPGQAPCSRGSSWSADLDKCMDCASCRARPHSDFCLGCAAAPPAPFRLLWPRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 278 aa, Homo Sapiens
APCSRGSSWSADLDKCMDCASCRARPHSDFCAGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 150 - protein, 278 aa, Homo Sapiens
APCSRGSSWSADLDKCMDCASCRARPHSDACLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 151 - protein, 278 aa, Homo Sapiens
APCSRGSSWSADLDKCMDCASCRARPHSAFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 152 - protein, 278 aa, Homo Sapiens
APCSRGSSWSADLDKCMDCASCRARPHADFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 153 - protein, 278 aa, Homo Sapiens
APCSRGSSWSADLDKCMDCASCRARPASDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 154 - protein, 278 aa, Homo Sapiens
APCSRGSSWSADLDKCMDCASCRARAHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 155 - protein, 278 aa, Homo Sapiens
APCSRGSSWSADLDKCMDCASCRAAPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 156 - protein, 240 aa, Homo Sapiens
APCSRGSSWSADLDKCMDCASCAARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKS

SEQ ID NO: 157 - protein, 278 aa, Homo Sapiens
APCSRGSSWSADLDKCMDCAACRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 158 - protein, 278 aa, Homo Sapiens
APCSRGSSWSADLDKCMACASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 159 - protein, 278 aa, Homo Sapiens
APCSRGSSWSADLDACMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 160 - protein, 278 aa, Homo Sapiens
APCSRGSSWSADLAKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 161 - protein, 278 aa, Homo Sapiens
APCSRGSSWSAALDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 162 - protein, 278 aa, Homo Sapiens
APCSRGSSWAADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 163 - protein, 278 aa, Homo Sapiens
APCSRGSSASADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 164 - protein, 278 aa, Homo Sapiens
APCSRGSAWSADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 165 - protein, 278 aa, Homo Sapiens
APCSRGASWSADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 166 - protein, 278 aa, Homo Sapiens
APCSAGSSWSADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 167 - protein, 278 aa, Homo Sapiens
APCARGSSWSADLDKCMDCASCRARPHSDFCLGCAIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 168 - protein, 53 aa, Homo Sapiens
EQAPGTAPCSRGSSWSADLDKCMDCASCRARPHSDFCLGCAAAPPAPFRLLWP

SEQ ID NO: 169 - protein, 129 aa, Homo Sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 170 - DNA, 959 bp, Homo Sapiens
atggctcggggctcgctgcgccggttgctgcggctcctcgtgctggggctctggctggcgttgctgcgctccgtggccggggagcaagcgccaggcaccgccccctgctcccgcggcagctcctggagcgcggacctggacaagtgcatggactgcgcgtcttgcagggcgcgaccgcacagcgacttctgcctgggctgcgctgcagcacctcctgcccccttccggctgctttggcccatccttgggggcgctctgagcctgaccttcgtgctggggctgctttctggctttttggtctggagacgatgccgcaggagagagaagttcaccacccccatagaggagaccggcggagagggctgcccagctgtggcgctgatccagtgacaatgtgccccctgccagccggggctcgcccactcatcattcattcatccattctagagccagtctctgcctcccagacgcggcgggagccaagctcctccaaccacaaggggggtggggggcggtgaatcacctctgaggcctgggcccagggttcaggggaaccttccaaggtgtctggttgccctgcctctggctccagaacagaaagggagcctcacgctggctcacacaaaacagctgacactgactaaggaactgcagcatttgcacaggggaggggggtgccctccttccttaggacctgggggccaggctgacttggggggcagacttgacactaggccccactcactcagatgtcctgaaattccaccacgggggtcaccctggggggttagggacctatttttaacactaggggctggcccactaggagggctggccctaagatacagacccccccaactccccaaagcggggaggagatatttattttggggagagtttggaggggagggagaatttattaataaaagaatctttaactttaaaaaaaaaaaaaaaaa

SEQ ID NO: 171 - protein, 5 aa, Homo Sapiens
  misc_feature (5)..(5): Xaa can be any naturally occurring amino acid
PYPMX

SEQ ID NO: 172 - protein, 17 aa, Homo Sapiens
  misc_feature (8)..(8): Xaa can be any naturally occurring amino acid
YISPSGGXTHYADSVKG

SEQ ID NO: 173 - protein, 11 aa, Homo Sapiens
GGDTYFDYFDY

SEQ ID NO: 174 - protein, 11 aa, Homo Sapiens
  misc_feature (8)..(8): Xaa can be any naturally occurring amino acid
RASQSISXYLN

SEQ ID NO: 175 - protein, 7 aa, Homo Sapiens
  misc_feature (1)..(1): Xaa can be any naturally occurring amino acid
XASSLQS

SEQ ID NO: 176 - protein, 10 aa, Homo Sapiens
  misc_feature (5)..(6): Xaa can be any naturally occurring amino acid
QQSYXXPXIT

SEQ ID NO: 177 - DNA, 645 bp, Homo Sapiens
gacatccagatgacccagagccccagcagcctgagcgcctccgtgggcgacagagtgaccatcacctgtcgggccagccagagcatcagcggctacctgaactggtatcagcagaagcccggcaaggcccccaagctgctgatctaccaggccagctccctgcagagcggcgtgccaagcagattcagcggcagcggctccggcaccgacttcaccctgaccatcagcagcctgcagcccgaggacttcgccacctactactgccagcagagctacaccagccccttcatcaccttcggccagggcaccaaggtggaaatcaagcggaccgtggccgctcccagcgtgttcatcttcccacccagcgacgagcagctgaagtccggcacagccagcgtggtctgcctgctgaacaacttctacccccgcgaggccaaggtgcagtggaaggtggacaacgccctgcagtccggcaactcccaggaaagcgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 178 - DNA, 1347 bp, Homo Sapiens
gaagttcaattgttagagtccggcggaggcctggtgcagcctggcggcagcctgagactgtcttgcgccgccagcggcttcacattcagcccctaccccatgatctgggtccgccaggctccaggcaagggcctggaatgggtgtcctacatcagccccagcggcggcagcacccactacgccgatagcgtgaagggccggttcaccatcagccgggacaacagcaagaacaccctgtacctgcagatgaacagcctgcgggccgaggacaccgccgtgtactattgcgccagaggcggcgacacctacttcgattacttcgactactggggccagggcaccctggtgacagtgtccagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 179 - DNA, 645 bp, Homo Sapiens
gacatccagatgacccagtctccagccaccctgtctgcatctgtaggagacagagtcaccatcacttgccgggcaagtcagagcattagcggctatttaaattggtatcagcagaaaccagggaaagcccctaagctcctgatctatcaggcatccagtttgcaaagtggggtcccatcaaggttcagtggcagtggatctgggacagatttcactctcaccatcagcagtctgcaacctgaagattttgcaacttactactgtcaacagagctacactagtccattcatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 180 - DNA, 1347 bp, Homo Sapiens
gaagttcaattgttagagtctggtggcggtcttgttcagcctggtggttctttacgtctttcttgcgctgcttccggattcactttctctccttaccctatgatctgggttcgccaagctcctggtaaaggtttggagtgggtttcttatatctctccttctggtggcaagactcattatgctgactccgttaaaggtcgcttcactatctctagagacaactctaagaatactctctacttgcagatgaacagcttaagggctgaggacacggccgtgtattactgtgcgagagggggtgatacttatttcgactactttgactactggggccagggaaccctggtcaccgtctcaagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 181 - DNA, 645 bp, Homo Sapiens
gacatccagatgacccagagccccagcagcctgagcgcctccgtgggcgacagagtgaccatcacctgtcgggccagccagagcatcagcagctacctgaactggtatcagcagaagcccggcaaggcccccaagctgctgatctaccaggccagctccctgcagagcggcgtgccaagcagattcagcggcagcggctccggcaccgacttcaccctgaccatcagcagcctgcagcccgaggacttcgccacctactactgccagcagagctacaccagccccttcatcaccttcggccagggcaccaaggtggaaatcaagcggaccgtggccgctcccagcgtgttcatcttcccacccagcgacgagcagctgaagtccggcacagccagcgtggtctgcctgctgaacaacttctacccccgcgaggccaaggtgcagtggaaggtggacaacgccctgcagtccggcaactcccaggaaagcgtgaccgagcaggacagcaaggactccacctacagcctgagcagcaccctgaccctgagcaaggccgactacgagaagcacaaggtgtacgcctgcgaagtgacccaccagggcctgtccagccccgtgaccaagagcttcaaccggggcgagtgc

SEQ ID NO: 182 - DNA, 1347 bp, Homo Sapiens
gaagttcaattgttagagtccggcggaggcctggtgcagcctggcggcagcctgagactgtcttgcgccgccagcggcttcacattcagcccctaccccatgatgtgggtccgccaggctccaggcaagggcctggaatgggtgtcctacatcagccccagcggcggcagcacccactacgccgatagcgtgaagggccggttcaccatcagccgggacaacagcaagaacaccctgtacctgcagatgaacagcctgcgggccgaggacaccgccgtgtactattgcgccagaggcggcgacacctacttcgattacttcgactactggggccagggcaccctggtgacagtgtccagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 183 - DNA, 645 bp, Homo Sapiens
gacatccagatgacccagtctccagccaccctgtctgcatctgtaggagacagagtcaccatcacttgccgggcaagtcagagcattagcagctatttaaattggtatcagcagaaaccagggaaagcccctaagctcctgatctatcaggcatccagtttgcaaagtggggtcccatcaaggttcagtggcagtggatctgggacagatttcactctcaccatcagcagtctgcaacctgaagattttgcaacttactactgtcaacagagctacactagtccattcatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 184 - DNA, 1347 bp, Homo Sapiens
gaagttcaattgttagagtctggtggcggtcttgttcagcctggtggttctttacgtctttcttgcgctgcttccggattcactttctctccttaccctatgatgtgggttcgccaagctcctggtaaaggtttggagtgggtttcttatatctctccttctggtggcaagactcattatgctgactccgttaaaggtcgcttcactatctctagagacaactctaagaatactctctacttgcagatgaacagcttaagggctgaggacacggccgtgtattactgtgcgagagggggtgatacttatttcgactactttgactactggggccagggaaccctggtcaccgtctcaagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 185 - DNA, 645 bp, Homo Sapiens
gacatccagatgacccagagccccagcagcctgtctgccagcgtgggcgacagagtgaccatcacctgtagagccagccagagcatcagcagctacctgaactggtatcagcagaagcccggcaaggcccccaagctgctgatctatgccgccagctctctgcagagcggagtgcccagcagattttctggcagcggcagcggcaccgacttcaccctgacaatcagcagcctgcagcccgaggacttcgccacctactactgccagcagagctacagcacccccggcatcacatttggccagggcaccaaggtggaaatcaagcggacagtggccgctcccagcgtgttcatcttcccacctagcgacgagcagctgaagtccggcacagccagcgtcgtgtgcctgctgaacaacttctacccccgcgaggccaaggtgcagtggaaggtggacaatgccctgcagtccggcaactcccaggaaagcgtcaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgt

SEQ ID NO: 186 - DNA, 1347 bp, Homo Sapiens
gaagttcaattgttagagtccggcggaggcctggtgcagcctggcggatctctgagactgagctgtgccgccagcggcttcaccttcagcccctaccctatgatgtgggtccgacaggcccctggcaagggactggaatgggtgtcctacatctctcccagcggcggcagcacccactacgccgattctgtgaagggccggttcaccatcagccgggacaacagcaagaacaccctgtacctgcagatgaacagcctgcgggccgaggacaccgccgtgtactattgtgccagaggcggcgacacctacttcgattacttcgactactggggccagggcaccctggtcaccgtgtcatctgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 187 - DNA, 645 bp, Homo Sapiens
gacatccagatgacccagtctccagccaccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagctactctagtccagggatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 188 - DNA, 1347 bp, Homo Sapiens
gaagttcaattgttagagtctggtggcggtcttgttcagcctggtggttctttacgtctttcttgcgctgcttccggattcactttctctccttaccctatgatgtgggttcgccaagctcctggtaaaggtttggagtgggtttcttatatctctccttctggtggcaagactcattatgctgactccgttaaaggtcgcttcactatctctagagacaactctaagaatactctctacttgcagatgaacagcttaagggctgaggacacggccgtgtattactgtgcgagagggggtgatacgtatttcgactactttgactactggggccagggaaccctggtcaccgtctcaagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 189 - DNA, 651 bp, Homo Sapiens
gcacaagacatccagatgacccagtctccagccaccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagctactctagtccagggatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 190 - DNA, 1347 bp, Homo Sapiens
gaagttcaattgttagagtctggtggcggtcttgttcagcctggtggttctttacgtctttcttgcgctgcttccggattcactttctctccttaccctatgatgtgggttcgccaagctcctggtaaaggtttggagtgggtttcttatatctctccttctggtggcaagactcattatgctgactccgttaaaggtcgcttcactatctctagagacaactctaagaatactctctacttgcagatgaacagcttaagggctgaggacacggccgtgtattactgtgcgagagggggtgatacgtatttcgactactttgactactggggccagggaaccctggtcaccgtctcaagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagcgggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggaagagatgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 191 - DNA, 651 bp, Homo Sapiens; identical to SEQ ID NO: 189

SEQ ID NO: 192 - DNA, 1347 bp, Homo Sapiens
gaagttcaattgttagagtctggtggcggtcttgttcagcctggtggttctttacgtctttcttgcgctgcttccggattcactttctctccttaccctatgatgtgggttcgccaagctcctggtaaaggtttggagtgggtttcttatatctctccttctggtggcaagactcattatgctgactccgttaaaggtcgcttcactatctctagagacaactctaagaatactctctacttgcagatgaacagcttaagggctgaggacacggccgtgtattactgtgcgagagggggtgatggttatttcgactactttgactactggggccagggaaccctggtcaccgtctcaagcgcctccaccaagggcccatcggtcttcccgctagcacccagcagcaagagcaccagcggcggaacagccgccctgggctgcctggtgaaagactacttccccgagcccgtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagcgggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggaagagatgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 193 - DNA, 651 bp, Homo Sapiens
gcacaagacatccagatgacccagtctccagccaccctgtctgcatctgtaggagacagagtcaccatcacttgccgggcaagtcagagcattagcggctatttaaattggtatcagcagaaaccagggaaagcccctaagctcctgatctataacgcatccagtttgcaaagtggggtcccatcaaggttcagtggcagtggatctgggacagatttcactctcaccatcagcagtctgcaacctgaagattttgcaacttactactgtcaacagagctacactagtccattcatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 194 - DNA, 1347 bp, Homo Sapiens; identical to SEQ ID NO: 180

SEQ ID NO: 195 - DNA, 651 bp, Homo Sapiens
gcacaagacatccagatgacccagtctccagccaccctgtctgcatctgtaggagacagagtcaccatcacttgccgggcaagtcagagcattagcagctatttaaattggtatcagcagaaaccagggaaagcccctaagctcctgatctataacgcatccagtttgcaaagtggggtcccatcaaggttcagtggcagtggatctgggacagatttcactctcaccatcagcagtctgcaacctgaagattttgcaacttactactgtcaacagagctacactagtccagggatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 196 - DNA, 1347 bp, Homo Sapiens; identical to SEQ ID NO: 184

SEQ ID NO: 197 - DNA, 651 bp, Homo Sapiens
gcacaagacatccagatgacccagtctccagccaccctgtctgcatctgtaggagacagagtcaccatcacttgccgggcaagtcagagcattagcggctatttaaattggtatcagcagaaaccagggaaagcccctaagctcctgatctataacgcatccagtttgcaaagtggggtcccatcaaggttcagtggcagtggatctgggacagatttcactctcaccatcagcagtctgcaacctgaagattttgcaacttactactgtcaacagagctacactagtccagggatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 198 - DNA, 1347 bp, Homo Sapiens; identical to SEQ ID NO: 184

SEQ ID NO: 199 - DNA, 651 bp, Homo Sapiens
gcacaagacatccagatgacccagtctccagccaccctgtctgcatctgtaggagacagagtcaccatcacttgccgggcaagtcagagcattagcagctatttaaattggtatcagcagaaaccagggaaagcccctaagctcctgatctataacgcatccagtttgcaaagtggggtcccatcaaggttcagtggcagtggatctgggacagatttcactctcaccatcagcagtctgcaacctgaagattttgcaacttactactgtcaacagagctacactagtccattcatcactttcggccctgggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 200 - DNA, 1347 bp, Homo Sapiens; identical to SEQ ID NO: 184

SEQ ID NO: 201 - DNA, 654 bp, Mus Musculus
gatatcgtgctgacacagtctcccgccagcctggccgtgtctctcggccagagagccaccatcagctgccgggccaacaagagcgtgtccaccagcagctacagctacatgcactggtatcagcagaagcccggccagccccccaagctgctgattaagtacgccagcaacctggaaagcggcgtgcccgccagattcagcggcagcggctctggcaccgacttcatcctgaacatccaccccgtggaagaagaggacgccgccacctactactgccagcacagcagagagctgcccttcaccttcggcagcggcaccaagctggaaatcaagcgggccgatgccgcccctaccgtgtccatcttcccacccagcagcgagcagctgaccagcggcggagccagcgtcgtgtgcttcctgaacaacttctaccccaaggacatcaacgtgaagtggaagatcgacggcagcgagcggcagaacggcgtgctgaacagctggaccgaccaggacagcaaggactccacctacagcatgagcagcaccctgaccctgaccaaggacgagtacgagcggcacaacagctacacatgcgaggccacccacaagaccagcaccagccccatcgtgaagtccttcaaccggaacgagtgc

SEQ ID NO: 202 - DNA, 1350 bp, Mus Musculus
caggtgcagctgcagcagtctggccctgaagtcgtgcggcctggcgtgtccgtgaagatcagctgcaagggcagcggctacaccttcaccgactacggcatccactgggtcaagcagagccacgccaagagcctggaatggatcggcgtgatcagcacctacaacggctacaccaactacaaccagaagttcaagggcaaggccaccatgaccgtggacaagagcagcagcaccgcctacatggaactggcccggctgaccagcgaggacagcgccatctactactgcgccagagcctactacggcaacctgtactacgccatggactactggggccagggcaccagcgtgaccgtgtcctctgccaagaccaccgcccctagcgtgtaccctctggcccctgtgtgtggcgacaccaccggcagctctgtgactctgggctgcctggtcaagggctacttccccgagcccgtgacactgacctggaacagcggcagcctgagcagcggcgtgcacacctttccagccgtgctgcagagcgacctgtacaccctgagcagctccgtgaccgtgacaagcagcacctggcccagccagagcatcacctgtaacgtggcccaccccgccagcagcaccaaggtggacaagaagatcgagcccagaggccccaccatcaagccctgccccccttgcaagtgcccagcccccaatctgctgggcggacccagcgtgttcatcttcccacccaagatcaaggacgtgctgatgatcagcctgagccccatcgtgacctgcgtggtggtggacgtgtccgaggacgaccccgacgtgcagatcagttggttcgtgaacaacgtggaagtgcacaccgcccagacccagacccacagagaggactacaacagcaccctgcgggtggtgtccgccctgcccatccagcaccaggactggatgagcggcaaagaattcaagtgcaaagtgaacaacaaggacctgcctgcccccatcgagcggaccatcagcaagcccaagggcagcgtgcgggctccccaggtgtacgtgctgcccccacccgaggaagagatgaccaagaagcaggtcacactgacctgcatggtcaccgacttcatgcccgaggacatctacgtggaatggaccaacaacggcaagaccgagctgaactacaagaacaccgagcctgtgctggacagcgacggcagctacttcatgtacagcaagctgcgggtggaaaagaaaaactgggtggaacggaacagctacagctgcagcgtggtgcacgagggcctgcacaaccaccacaccaccaagagcttcagccggacccccggc

SEQ ID NO: 203 - DNA, 654 bp, Mus Musculus
gatatcgtgctgacacagagccccgccagcctgaccgtgtctctcggccagagagccaccatcagctgccgggccagccagagcgtgtccaccagcagctacagctacatgcagtggtatcagcagcggcctggccagccccccaagctgctgattaagtacgccaccaacctggacagcggcgtgcccgccagattttctggcagcggcagcggcacagacttcaccctgaacatccaccccgtggaagaagaggacgccgccacctactactgccagcacagctgggagatcccttacaccttcggcggaggcaccaagctggaaatcaagcgggccgatgccgcccctaccgtgtccatcttcccacccagcagcgagcagctgaccagcggcggagccagcgtcgtgtgcttcctgaacaacttctaccccaaggacatcaacgtgaagtggaagatcgacggcagcgagcggcagaacggcgtgctgaacagctggaccgaccaggacagcaaggactccacctacagcatgagcagcaccctgaccctgaccaaggacgagtacgagcggcacaacagctacacatgcgaggccacccacaagaccagcaccagccccatcgtgaagtccttcaaccggaacgagtgc

SEQ ID NO: 204 - DNA, 1344 bp, Mus Musculus
gaagtgaagctggaagagtctggcggcggactggtccagcctggcggcagcatgaagctgagctgcgtggccagcggcttcaccttcaacaactactggatgagctgggtccgacagagccccgagaagggcctggaatggctggccgagatccggctgaagtccgacaactacgccacccactacgccgagagcgtgaagggcaagttcaccatcagccgggacgacagcaagagccggctgtacctgcagatgaacaacctgcgggccgagaacaccggcatctactactgcaccggcggcttcgccgactacttcgactactggggccagggcaccaccctgaccgtgtcctctgccaagaccaccgcccctagcgtgtaccctctggcccctgtgtgtggcgacaccaccggcagctctgtgactctgggctgcctggtcaagggctacttccccgagcccgtgacactgacctggaacagcggcagcctgagcagcggcgtgcacacctttccagccgtgctgcagagcgacctgtacaccctgagcagctccgtgaccgtgacaagcagcacctggcccagccagagcatcacctgtaacgtggcccaccccgccagcagcaccaaggtggacaagaagatcgagcccagaggccccaccatcaagccctgccccccttgcaagtgcccagcccccaatctgctgggcggacccagcgtgttcatcttcccacccaagatcaaggacgtgctgatgatcagcctgagccccatcgtgacctgcgtggtggtggacgtgtccgaggacgaccccgacgtgcagatcagttggttcgtgaacaacgtggaagtgcacaccgcccagacccagacccacagagaggactacaacagcaccctgcgggtggtgtccgccctgcccatccagcaccaggactggatgagcggcaaagaattcaagtgcaaagtgaacaacaaggacctgcctgcccccatcgagcggaccatcagcaagcccaagggcagcgtgcgggctccccaggtgtacgtgctgcccccacccgaggaagagatgaccaagaagcaggtcacactgacctgcatggtcaccgacttcatgcccgaggacatctacgtggaatggaccaacaacggcaagaccgagctgaactacaagaacaccgagcctgtgctggacagcgacggcagctacttcatgtacagcaagctgcgggtggaaaagaaaaactgggtggaacggaacagctacagctgcagcgtggtgcacgagggcctgcacaaccaccacaccaccaagagcttcagccggacccccggc

SEQ ID NO: 205 - DNA, 654 bp, Homo Sapiens
gacattgtgctgacacagtctcctgcttccctggctgtatctctggggcagagggccaccatctcatgcagggccagcaaaagtgtcagtacatctagctatagttatatgcactggtaccaacagaaaccaggacagccacccaaactcctcatcaaatatgcatccaacctagaatctggggtccctgccaggttcagtggcagtgggtctgggacagacttctccctcaacatccatcccatggaggaggacgataccgcaatgtatttctgtcagcacagtagggagcttccattcacgttcggcggagggacaaagttggaaataaaacgtacggtggccgctcccagcgtgttcatcttcccacccagcgacgagcagctgaagtccggcaccgccagcgtcgtgtgcctgctgaacaacttctacccccgcgaggccaaggtgcagtggaaggtggacaacgccctgcagagcggcaacagccaggaaagcgtcaccgagcaggacagcaaggactccacctacagcctgtccagcaccctgaccctgagcaaggccgactacgagaagcacaaggtgtacgcctgcgaagtgacccaccagggcctgagcagccccgtgaccaagagcttcaaccggggcgagtgc

SEQ ID NO: 206 - DNA, 1350 bp, Homo Sapiens
caggtccagctggtgcagtctggggctgaggtgaagaagcctggggcctcagtgaaggtttcctgcaagggttccggctacacattcactgattatggcatgcactgggtgcggcaggcccctggacaagggctagagtggatgggagttattagtacttacaatggttatacaaactacaaccagaagtttaagggcagagtcacaatgactgtagacaaatccacgagcacagcctatatggaacttcggagcttgagatctgacgatacggccgtgtattactgtgcaagagcctactatggcaacctttactatgctatggactactggggtcaaggaaccctggtcaccgtctcctcagctagcaccaaaggcccgagcgtgtttccgctggccccgagcagcaagagcaccagcggcggaacagccgccctgggctgcctggtgaaagactacttccccgaaccggtgaccgtgtcctggaactctggcgccctgaccagcggagtgcataccttccccgccgtgctgcagagcagcggcctgtacagcctgagcagcgtggtgacagtgcccagcagcagcctgggaacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggacccagaagtgaagtttaattggtacgtggacggcgtggaagtgcataacgccaagaccaagcccagagaggaacagtacaacagcacctaccgggtggtgtccgtgctgaccgtgctgcaccaggactggctgaacggcaaagagtacaagtgcaaggtctccaacaaggccctgcctgcccccatcgagaaaaccatcagcaaggccaagggccagccccgcgagcctcaggtgtacacactgccccccagccgggatgagctgaccaagaaccaggtgtccctgacctgtctggtgaaaggcttctaccccagcgatatcgccgtggaatgggagagcaacggccagcccgagaacaattacaagaccaccccccctgtgctggacagcgacggctcattcttcctgtactccaagctgaccgtggacaagagccggtggcagcagggcaacgtgttcagctgcagcgtgatgcacgaggccctgcacaatcactacacccagaagtccctgagcctgagccccggc

SEQ ID NO: 207 - DNA, 654 bp, Homo Sapiens
gatatccagatgacccagagccccagcagcctgagcgccagcgtgggcgacagagtgaccatcacctgtcgggccagccagagcgtgtccaccagcagctacagctacatgcactggtatcagcagaagcccggcaaggcccccaagctgctgattaagtacgccagcaacctggaaagcggcgtgcccagccggtttagcggctctggcagcggcaccgacttcaccctgaccatcagcagtctgcagcccgaggacttcgccacctactactgccagcacagctgggagatcccttacaccttcggcggaggcaccaaggtggaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgtctaaagcagactacgagaaacacaaagtctacgcctgcgaagtcacccatcagggcctgagctcgcccgtcacaaagagcttcaacaggggagagtgt

SEQ ID NO: 208 - DNA, 1347 bp, Homo Sapiens
caggtggaattggtggaaagcggcggaggcctggtgcagcctggcggaagcctgagactgagctgtgccgccagcggcttcaccttcagcagctactggatgagctgggtccgacaggctccaggcaagggcctggaatgggtggccgagatccggctgaagtccgacaactacgccacccactacgccgagagcgtgaagggccggttcaccatcagccgggacgacagcaagaacagcctgtacctgcagatgaacagcctgcgggccgaggacaccgccgtgtactactgcaccggctactacgccgacgccatggactactggggccagggcaccctggtcaccgtcagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 209 - DNA, 654 bp, Mus Musculus
gatatcgtgctgacacagtctcccgccagcctggccgtgtctctcggccagagagccaccatcagctgcaaggccagccagagcgtgtccaccagcacctacagctacatgcagtggtatcagcagcggcctggacagagccccaagctgctgattaagtacgccagcaagctggacagcggcgtgcccgccagattttctggcagcggcagcggcaccgacttcaccctgaacatccaccccgtggaagaagaggacaccgccacctactactgccagcacagctgggagctgccctacaccttcggcggaggcacccggctggaaatcaagagggccgatgccgcccctaccgtgtccatcttcccacccagcagcgagcagctgaccagcggcggagccagcgtcgtgtgcttcctgaacaacttctaccccaaggacatcaacgtgaagtggaagatcgacggcagcgagcggcagaacggcgtgctgaacagctggaccgaccaggacagcaaggactccacctacagcatgagcagcaccctgaccctgaccaaggacgagtacgagcggcacaacagctacacatgcgaggccacccacaagaccagcaccagccccatcgtgaagtccttcaaccggaacgagtgc

SEQ ID NO: 210 - DNA, 1344 bp, Mus Musculus
gaagtgaagctgggagagtctggcggcggactggtccagcctggcggcagcatgaagctgagctgcgtggccagcggcttcccattcaccaaatactggatgaactgggtccgacagagccccgagaagggcctggaatgggtggccgagatccggctgaagtccgacaactacgccacccactacgccgagagcgccaagggccggttcaccatcagccgggacgacagccggtccagcgtgtacctgcagatgaacaacctgcgggccgaggacaccgccatctactactgcagccccacctatgccgacaccatggactactggggccagggcaccagcgtgacagtgtccagcgccaagaccaccgcccctagcgtgtaccctctggcccctgtgtgtggcgacaccaccggcagctctgtgactctgggctgcctggtcaagggctacttccccgagcccgtgacactgacctggaacagcggcagcctgagcagcggcgtgcacacctttccagccgtgctgcagagcgacctgtacaccctgagcagctccgtgaccgtgacaagcagcacctggcccagccagagcatcacctgtaacgtggcccaccccgccagcagcaccaaggtggacaagaagatcgagcccagaggccccaccatcaagccctgccccccttgcaagtgcccagcccccaatctgctgggcggacccagcgtgttcatcttcccacccaagatcaaggacgtgctgatgatcagcctgagccccatcgtgacctgcgtggtggtggacgtgtccgaggacgaccccgacgtgcagatcagttggttcgtgaacaacgtggaagtgcacaccgcccagacccagacccacagagaggactacaacagcaccctgcgggtggtgtccgccctgcccatccagcaccaggactggatgagcggcaaagaattcaagtgcaaagtgaacaacaaggacctgcctgcccccatcgagcggaccatcagcaagcccaagggcagcgtgcgggctccccaggtgtacgtgctgcccccacccgaggaagagatgaccaagaagcaggtcacactgacctgcatggtcaccgacttcatgcccgaggacatctacgtggaatggaccaacaacggcaagaccgagctgaactacaagaacaccgagcctgtgctggacagcgacggcagctacttcatgtacagcaagctgcgggtggaaaagaaaaactgggtggaacggaacagctacagctgcagcgtggtgcacgagggcctgcacaaccaccacaccaccaagagcttcagccggacccccggc

SEQ ID NO: 211 - DNA, 654 bp, Mus Musculus
gatatcgtgctgacacagtctcccgccagcctggccgtgtctctcggccagagagccaccatcagctgccgggccagcaagagcgtgtccaccagcagctacagctacatgcactggtatcagcagaagcccggccagccccccaagctgctgatcaagtacaccagcaacctggaaagcggcgtgcccgccagattcagcggaagcggctccggcaccgacttcatcctgaacatccaccccgtggaagaagaggacgccgccacctactactgccagcacagcagagagctgccctggaccttcggcggaggcaccaagctggaaatcaagcgggccgatgccgcccctaccgtgtccatcttcccacccagcagcgagcagctgaccagcggcggagccagcgtcgtgtgcttcctgaacaacttctaccccaaggacatcaacgtgaagtggaagatcgacggcagcgagcggcagaacggcgtgctgaacagctggaccgaccaggacagcaaggactccacctacagcatgagcagcaccctgaccctgaccaaggacgagtacgagcggcacaacagctacacatgcgaggccacccacaagaccagcaccagccccatcgtgaagtccttcaaccggaacgagtgc

SEQ ID NO: 212 - DNA, 1353 bp, Mus Musculus
caggtgtccctgaaagagagcggccctggcatcctgcagcctagccagaccctgagcctgacctgcagcttcagcggcttcagcctgagcaccagcggcatgggcgtgtcctggatcagacagcccagcggcaagggcctggaatggctggcccacatctactgggacgacgacaagcggtacaaccccagcctgaagtcccggctgaccatctccaaggacaccagccggaatcaggtgttcctgaagatcaccagcgtggacaccgccgataccgccacctactactgcgccagaagaggccccgactactacggctactaccccatggactattggggccagggcaccagcgtgaccgtgtctgccaagaccaccgcccctagcgtgtaccctctggcccctgtgtgtggcgacaccaccggcagctctgtgactctgggctgcctggtcaagggctacttccccgagcccgtgacactgacctggaacagcggcagcctgagcagcggcgtgcacacctttccagccgtgctgcagagcgacctgtacaccctgagcagctccgtgaccgtgacaagcagcacctggcccagccagagcatcacctgtaacgtggcccaccccgccagcagcaccaaggtggacaagaagatcgagcccagaggccccaccatcaagccctgccccccttgcaagtgcccagcccccaatctgctgggcggacccagcgtgttcatcttcccacccaagatcaaggacgtgctgatgatcagcctgagccccatcgtgacctgcgtggtggtggacgtgtccgaggacgaccccgacgtgcagatcagttggttcgtgaacaacgtggaagtgcacaccgcccagacccagacccacagagaggactacaacagcaccctgcgggtggtgtccgccctgcccatccagcaccaggactggatgagcggcaaagaattcaagtgcaaagtgaacaacaaggacctgcctgcccccatcgagcggaccatcagcaagcccaagggcagcgtgcgggctccccaggtgtacgtgctgcccccacccgaggaagagatgaccaagaagcaggtcacactgacctgcatggtcaccgacttcatgcccgaggacatctacgtggaatggaccaacaacggcaagaccgagctgaactacaagaacaccgagcctgtgctggacagcgacggcagctacttcatgtacagcaagctgcgggtggaaaagaaaaactgggtggaacggaacagctacagctgcagcgtggtgcacgagggcctgcacaaccaccacaccaccaagagcttcagccggacccccggc

SEQ ID NO: 213 - protein, 449 aa, Homo Sapiens
EVQLLESGGGLVQPGGSLRLSCAASGFTFSPYPMIWVRQAPGKGLEWVSYISPSGGSTHYADSVKGRFTISRDNSKNTLYLQMNSLRAEDTAVYYCARGGDTYFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG